(19) United States
(12) Patent Application Publication
(10) Pub. No.: US 2018/0258428 A1
(43) Pub. Date: Sep. 13, 2018

(54) METHODS AND COMPOSITIONS FOR THE TREATMENT OF A METABOLIC DISORDER

(71) Applicant: Children's Hospital Medical Center, Cincinnati, OH (US)

(72) Inventor: Takahisa Nakamura, Cincinnati, OH (US)

(21) Appl. No.: 15/916,388

(22) Filed: Mar. 9, 2018

Related U.S. Application Data
(60) Provisional application No. 62/468,972, filed on Mar. 9, 2017.

(51) Int. Cl. C12N 15/113; A61P 1/16

(52) U.S. Cl. CPC C12N 15/113; C12N 2310/141; A61P 1/16

(57) ABSTRACT

Disclosed herein are methods and compositions for the treatment of metabolic related disorders. The methods and compositions may be useful for improving systemic glucose metabolism in the liver on an individual in need thereof, via administration of administering a therapeutically effective amount of an agent that interferes with Ago2 activity and/or function in combination with a pharmaceutically acceptable excipient.

### CROSS-REFERENCE TO RELATED APPLICATIONS

This application claims priority to and benefit of U.S. Provisional Application 62/468,972, filed Mar. 9, 2017, the contents of which are incorporated in its entirety for all purposes.

### BACKGROUND

The worldwide prevalence of obesity has reached pandemic proportions, bringing with it a host of associated diseases, such as type 2 diabetes (T2D), non-alcoholic steatohepatitis (NASH), and cancer^{1,2}. Despite extensive efforts to address the issue of obesity and associated disease states, there remains a need in the art for treatments that can address this need in the art. The instant disclosure seeks to address one or more of the aforementioned needs in the art.

### BRIEF SUMMARY

Disclosed herein are methods and compositions for the treatment of metabolic related disorders.

### BRIEF DESCRIPTION OF THE DRAWINGS

This application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIGS. 1A-1G. Effects of hepatic Ago2-deficiency on MD-miRNA expression in the liver. (FIGS. 1A, 1B, and 1C) A heatmap diagram illustrating the differential expression of hepatic mature miRNAs (FIG. 1A), raw counts of significant miRNAs normalized by DESeq2 and transformed by Log2 (FIG. 1B), and relative levels of significant miRNAs whose expression levels are in the top 10 percent in the WT liver (FIG. 1C) in the liver of L-Ago2 WT (n=3) and L-Ago2 KO (n=3) mice fed NCD at 9 weeks of age. Significant miRNAs differentially expressed between L-Ago2 WT and L-Ago2 KO groups were identified using DESeq2 (|fold change|>2× and adjusted p<0.05) and plotted as a heatmap using z-score. (FIG. 1D) Metabolic pathway enrichment analysis of miRNAs significantly down-regulated (blue) and up-regulated (red) miRNAs in the liver of L-Ago2 KO mice fed NCD at 9 weeks of age. These miRNAs were queried to calculate the most enriched KEGG pathways using DIANA-mirPath web-server (p<0.05 and MicroT threshold<0.8). Pathways unrelated to hepatic functions were excluded in this pyramid plot. (FIGS. 1E, 1F and 1G) Expression levels of MD-miRNAs' target mRNAs (FIG. 1E), selective MD-miRNAs (FIG. 1F), and their pri-miRNAs (FIG. 1G) in the liver of L-Ago2 WT (n=8) and L-Ago2 KO (n=8) mice fed NCD at 25 weeks of age. Data are shown as the mean±SEM, *p<0.05, **p<0.01.

FIGS. 2A-2G. Hepatic Ago2-deficnecy improves glucose metabolism with enhanced pyruvate oxidation. (FIG. 2A) Glucose tolerance test performed in L-Ago2 WT (n=10) and KO (n=8) mice fed NCD at 20 weeks of age. (FIG. 2B) Insulin tolerance test performed in L-Ago2 WT (n=9) and KO (n=6) mice fed NCD at 21 weeks of age. (FIG. 2C) Pyruvate tolerance test performed in L-Ago2 WT (n=9) and KO (n=10) mice fed NCD at 24 weeks of age. (FIG. 2D) Glucose production in primary hepatocytes incubated in the absence or presence of 100 or 200 μM Bt-cAMP or pCPT-cAMP. (FIG. 2E) Extracellular acidification (ECAR) in the absence or presence of 10 mM glucose in primary hepatocytes isolated from L-Ago2 WT and L-Ago2 KO mice. (FIG. 2F) Mitochondrial oxygen consumption rate (OCR) in the absence or presence of 2 mM pyruvate in primary hepatocytes isolated from L-Ago2 WT and L-Ago2 KO mice. (FIG. 2G) The ATP/ADP ratio in primary hepatocytes incubated in the absence or presence of 5 mM pyruvate. Data are shown as the mean±SEM, 857 *p<0.05, **p<0.01.

FIGS. 3A-3F. Hepatic Ago2-deficiency prevents S961-induced acute glucose intolerance. (FIG. 3A) Glucose tolerance tests at one-week post treatment of S961 or Phosphate-buffered saline (PBS). L-Ago2 WT (n=10 for PBS and n=13 for S961) and KO (n=11 for PBS and n=14 for S961) mice fed NCD at 9 weeks of age were continuously treated with S961 (10 nM/week) via osmotic pumps. The graph on the right shows an integrated area under the glucose disposal curves (AUC) for each condition. (FIG. 3B) Serum insulin levels after daytime food withdrawal for 6 hours in L-Ago2 WT and KO mice at 2-weeks post S961 (n=7, each genotype) or PBS (n=6, each genotype) treatment. (FIG. 3C) Hepatic glycogen contents in L-Ago2 WT and KO mice at 2-weeks post S961 (n=7, each genotype) or PBS (n=5, each genotype) treatment. (FIG. 3D) A heatmap diagram illustrating the differential expression of hepatic mature miRNAs in the liver of L-Ago2 WT and KO mice treated with PBS or S961 for 2 weeks. Significant miRNAs differentially expressed between genotypes were identified using DESeq2 (|fold change|>1.25× and adjusted p<0.0005). Clusters I and IV are miRNAs differentially expressed between L-Ago2 WT (n=6) and L-Ago2 872 KO (n=6) groups. Clusters II and III are miRNAs differentially expressed by S961 treatment in WT and KO groups, respectively. The log2 expression values were scaled by z-score. (FIGS. 3E and 3F) Effect of S961-treatment on expression of MD-miRNA (FIG. 3E) and genes regulating energy metabolism (FIG. 3F) in the liver of L-Ago2 WT and KO mice treated with PBS or S961 (n=6, each group) for 2 weeks. Data are shown as the mean±SEM, *p<0.05, **p<0.01.

FIGS. 4A-4Q. Ago2-deficiency in the liver prevents obesity-associated pathophysiology. (FIG. 4A) Body weights of L-Ago2 WT (n=16) and KO (n=17) mice fed HFD, L-Ago2 WT (n=15) and KO (n=11) mice fed CD, starting in 4 weeks of age. (FIG. 4B) Glucose tolerance test performed in L-Ago2 WT (n=16) and KO (n=17) mice fed HFD at 20 weeks of age. (FIG. 4C) Insulin tolerance test performed in L-Ago2 WT (n=16) and KO (n=17) mice fed HFD at 14 weeks of age. (FIG. 4D) Pyruvate tolerance test of L-Ago2 WT (n=8) and KO (n=8) mice fed HFD at 17 weeks of age. (FIGS. 4E-4H) Hyperinsulinemic-euglycemic clamp studies performed in L-Ago2 WT (n=6) and L-Ago2 KO (n=10) mice fed HFD for 20 weeks. (FIG. 4E) Glucose infusion rates (GIR) throughout the clamp procedures. The graph on the right shows an integrated area under curves (AUC) of GIR. (FIG. 4F) Insulin clearance levels (FIG. 4G) Tissue glucose uptakes in gastrocnemius muscle, visceral fat, and subcutaneous fat tissues. (FIG. 4H) Suppression of hepatic glucose production (sHGP) during the clamp. (FIG. 4I) Liver weight in L-Ago2 WT fed CD (n=5), L-Ago2 KO fed CD (n=4), L-Ago2 WT fed HFD (n=5), and L-Ago2 KO fed HFD (n=5) at 30 weeks of age. (FIGS. 4J and 4K) Liver triglyceride (TG) content (FIG. 4J) and serum ALT levels (FIG. 4K) in L-Ago2 WT fed HFD (n=7), and L-Ago2 KO (n=5) fed HFD at 23 weeks of age. (FIG. 4L) H&E-stained sections of the liver in each genotype at 30 weeks of age. (FIG. 4M) Expression levels of key mRNAs involved in energy metabolism in the liver of L-Ago2 WT (n=8) and L-Ago2 KO (n=5) mice fed HFD at 23 weeks of age. (FIG. 4N) Levels of (3-oxidation in the presence of 0.12 mM palmitate and mitochondrial OCR in the presence of 5 mM acetate in primary hepatocytes isolated from L-Ago2 WT and L-Ago2 KO mice. (FIG. 4O) Effects of a 14 hours fast on fat mass, and lean body mass in L-Ago2 WT (n=9) and KO (n=10) mice fed HFD at 20 weeks of age. (FIG. 4P) Copy numbers of mtDNA in L-Ago2 WT (n=7) and KO (n=5) mice fed HFD at 23 weeks of age. (FIG. 4Q) Energy expenditure in L-Ago2 WT (n=8) and KO (n=8) mice fed HFD for 12 weeks. Data are shown as the mean±SEM, *p<0.05, **p<0.01.

FIGS. 5A-5I. Hepatic Ago2 regulates expression of MD-miRNAs and Ampka1 in the pathogenesis of obesity. (FIG. 5A) A heatmap diagram illustrating the differential expression of hepatic mature miRNAs in the liver of L-Ago2 WT (n=3) and L-Ago2 KO (n=3) mice fed HFD for 16 weeks. Significant miRNAs differentially expressed between L-Ago2 WT and L-Ago2 KO groups were identified using DESeq2 (Ifold changel>2× and adjusted p<0.05) and plotted as a heatmap using z-score. (FIG. 5B) Metabolic pathway enrichment analysis of miRNAs significantly down-regulated (blue) and up-regulated (red) miRNAs in the liver of L-Ago2 KO mice fed HFD for 16 weeks. These miRNAs were queried to calculate the most enriched KEGG pathways using DIANA-mirPath web-server (p<0.05 and MicroT threshold<0.8). Pathways unrelated to liver functions were excluded in this pyramid plot. (FIG. 5C) Expression levels of specific MD-miRNAs in the liver of WT mice (n=8) fed NCD at 25 weeks of age, and WT (n=7) and L-Ago2 KO (n=5) mice fed HFD at 23 weeks of age. (FIG. 5D) Expression levels of MD-miRNAs' target mRNAs and pri-miRNAs in the liver of L-Ago2 WT (n=7) and L-Ago2 KO (n=5) mice fed HFD at 25 weeks of age. (FIG. 5E) Ago2 PAR-CLIP analysis of mouse bone marrow-derived macrophage. Ago2 PAR-CLIP reads shown in blue, overlap with multiple distinct, high-confidence miRNA binding sites. The Ampka1 (Prkaal) transcript is indicated by blue boxes (the wider box indicates the coding region and the narrower box indicates the 3′ UTR). RefSeq, reference sequence database. TargetScan 7.1 was used to identify potential binding sites for miRNAs whose expression levels changed in the Ago2-deficient conditions. (FIG. 5F) Compared expression levels of Ampka1, miR-148/152, and their pri-miRNAs in primary hepatocytes isolated from L-Ago2 WT and L-Ago2 KO mice. (FIG. 5G) Relative luciferase activity by which Ampka1 3′ UTR with or without harboring a mutation at miR148/152 putative target site was assessed in primary hepatocytes isolated from L-Ago2 WT and L-Ago2 KO mice. (FIG. 5H) Quantification of Ampka1, Cs, and β-actin mRNA levels in fractions collected from polysome profiles of primary hepatocytes isolated from L-Ago2 WT and L-Ago2 KO mice. The graphs show the quantification of the results. (FIG. 5I) A proposed role of hepatic Ago2 in the regulation of glucose and lipid metabolism in the liver. Data are shown as the mean±SEM. *p<0.05, **p<0.01.

FIGS. 6A-6I. Hepatic Ago2-deficiency enhances energy expenditure associated with protein synthesis and AMPK activation. (FIGS. 6A and 6B) Western blot analyses of AMPK expression and activation (FIG. 6A) and mRNA expression of the Tfam-mitochondrial genes (FIG. 6B) in the liver of L-Ago2 WT and L-Ago2 KO fed at HFD for 21 weeks. (FIG. 6C) ATP, ADP, and ATP/ADP ratio levels in L-Ago2 WT (n=5) and L-Ago2 KO (n=5) mice fed HFD at for 21 weeks. ATP/ADP ratio levels were independently measured with a distinct procedure from the ATP and ADP assays. (FIG. 6D) Western blot analysis of total and specific protein levels normalized by 12S-genomic DNA in the liver of L-Ago2 WT (n=5) and KO (n=5) mice fed HFD for 26 weeks. (FIG. 6E) Serum albumin levels in L-Ago2 WT (n=8) and L-Ago2 KO (n=8) mice fed HFD at 25 weeks of age. (FIG. 6F) Energy consumption rate measured in primary hepatocytes isolated from L-Ago2 WT and L-Ago2 KO mice in the presence of 1 mM metformin. (FIG. 6G) Effect of Ago2-deficiency on expression of AMPK activation in primary hepatocytes isolated from L-Ago2 WT and L-Ago2 KO mice in the presence of 1 mM metformin. The graphs show the quantification of the results. (FIG. 6H) Effect of Ago2 on nascent protein synthesis. Primary hepatocytes isolated from L-Ago2 WT and L-Ago2 KO mice were treated with or without 200 μM Phenformin (Phen) or 10 μM Rotenone (Rote) for 5 hours. (FIG. 6I) A proposed role of hepatic Ago2 in suppression of protein translation, leading to AMPK activation. The graphs show the quantification of the results. Data are shown as the mean±SEM. *p<0.05, **p<0.01.

FIGS. 7A-7N. Effects of hepatic Ago2-deficiency on glucose metabolism in liver-specific Ampka1-deficinet mice and in metformin treatment. (FIG. 7A) Glucose tolerance tests performed in L-Ampka1 WT (n=8), L-Ampka1 KO (n=8), and L-DKO (n=3) mice fed HFD for 5 weeks. (FIGS. 7B-F) Body weight (FIG. 7B), levels of blood glucose (FIG. 7C) and plasma insulin (FIG. 7D), and calculated HOMA IR (FIG. 7E) and insulin sensitivity (FIG. 7F) in L-Ampka1 WT (n=8), L-Ampka1 KO (n=8), and L-DKO (n=3) mice fed HFD for 6 weeks. (FIG. 7G) Expression levels 956 of miRNAs, pri-miRNAs, and miRNAs in the liver of L-Ampka1 WT (n=8), L-Ampka1 KO (n=8), and L-DKO (n=3) mice fed HFD for 8 weeks. (FIG. 7H) Blood glucose levels following a daytime food withdrawal for 6 hours in L-Ago2 WT (n=9) and L-Ago2 KO (n=11) mice fed HFD for 14 weeks of age before and after oral treatment of metformin (Met) (80 mg/kg/day) for 7 days. (FIG. 71) Expression levels of key genes in metformin's action in the liver of L-Ago2 WT (n=8) and L-Ago2 KO (n=5) mice fed HFD at 23 weeks of age. (FIG. 7J) Glucose lowering effect of metformin in an acute insulin resistance condition. L-Ago2 WT (n=13 for S961 and n=10 for S961 with metformin) and KO mice (n=14 for S961 and n=7 for S961 with metformin) fed NCD at 9 weeks of age were continuously treated with S961 (10 nM/week) together with or without Met (150 mg/kg/day) via osmotic pumps. Data of S961 treatment group is corresponding to one shown in FIG. 3A. Glucose tolerance test was performed in these mice at one week post metformin treatment. The graph on the right shows an integrated area under the glucose disposal curves (AUC) for each condition. (FIGS. 7K and 7L) Serum insulin levels (FIG. 7K) and hepatic glycogen contents (FIG. 7L) following a daytime food withdrawal for 6 hours in L-Ago2 WT and KO mice at 2-weeks post S961 with (n=8, each genotype) or without (n=7, each genotype) metformin treatment. (FIG. 7M) Expression levels of MD-miRNA in the liver of S961-treated or S961 plus metformin-treated L-Ago2 WT and KO mice (n=6, each group) at 2 weeks post treatment. Data are shown as the mean±SEM, *p<0.05, **p<0.01. (FIG. 7N) A proposed molecular mechanism by which hepatic Ago2-deficiency improves energy metabolism in the pathogenesis of obesity. Ago2-mediated miRNA biogenesis and RNA silencing coordinately suppress mitochondrial oxidation and protein translation, which result in lowered energy supply and consumption. Conversely, hepatic Ago2-deficiency enhances generation of energy from glucose and lipid for protein synthesis, leading to higher ADP and AMP amounts. As a result, the AMPK pathway is activated, leading to improvement of mitochondrial functions.

FIGS. 8A-8D. Generation of liver-specific Ago1- (L-Ago1 KO) and Ago2-deficient (L-Ago2 KO) mice. (FIGS. 8A and 8B) Western blot analyses of Ago1, Ago2, and Dicer in the liver of L-Ago1 KO (FIG. 8A) and L-Ago2 KO (FIG. 8B) mice fed NCD at 25 weeks of age. The graphs below show the quantification of the results. (FIGS. 8C and 8D) Serum ALT levels of L-Ago1 WT (n=7), L-Ago1 KO (n=4), L-Ago2 WT (n=5), and L-Ago2 KO (n=6) mice fed NCD at 9 weeks of age. Data are shown as the mean±SEM. *p<0.05

FIGS. 9A-9I. Analyses of miRNAs regulated by Ago2 in the liver. (FIG. 9A) Raw counts of miRNA Seq were normalized using DESeq2 and transformed by Log2 to show top 15 miRNAs in the liver of L-Ago2 WT and L-Ago2 KO mice fed NCD at 9 weeks of age. The most abundant miRNAs (top-25) are presented (*p<0.01, **p<0.001). (FIG. 9B) The effect of Ago2 WT or mutant reconstitution on MD-miRNAs in Ago2-deficient MEFs. Ago2-deficient MEFs were reconstituted with Ago2 variants. A panel below shows expression levels of Ago2 variants analyzed by western blotting. V: vector, WT: Ago2 WT, DA: Ago2 D669A mutant. (FIG. 9C) Expression levels of MD-miRNAs were assessed in Dicer^{+/-} and Dicer^{-/-} MEFs. (FIG. 9D) List of miRNAs whose expression levels are statistically reduced in the liver of L-Ago2 KO compared L-Ago2 WT mice and their structure analysis. There are mainly four proposed characteristics of miRNAs processed by Ago2 as follows: 1) loop size is less than 10 nt, 2) perfect matching at position 10 and 11 between guide and passenger strands, 3) long stem length (distance from 3′ to the loop is more than 30 nt), and 4) no 3′ hang out. These characteristics were evaluated to be scored as listed. (FIGS. 9E and 9F) Proportion (FIG. 9E) and frequency (FIG. 9F) of miRNAs significantly reduced in the liver of L-Ago2 KO mice with different total points. Alignments of read sequences that match miR-802-5p, miR-107-3p, or miR-103-3p with their read counts in the liver of L-Ago2 WT and KO mice fed NCD at 9 weeks of age. Sequence reads that completely matched to at least mature miRNAs registered in miRBase were extracted. These sequences were aligned using clustalX 2.1 with default option based on corresponding to precursor sequences. Read sequences that not completely matched to corresponding to precursor sequences are shown in red. Underlined sequences indicate mature miRNAs. There is no obvious processing rule, including addition of “U” and “A” at the 3′ end, of Ago2 against MD-miRNAs, except for reduced read count in L-Ago2 KO condition.

FIGS. 10A-10C. Effects of S961-treatment on inflammatory responses. (FIG. 10A) A scheme of S961-induced glucose intolerance. The S961 is administrated to mice fed NCD via osmotic pump. (FIG. 10B) JNK activation levels at 2 weeks post S961 treatment (10 nM/week). Activation levels of JNK were detected by anti-phospho-JNK antibody in lysates of liver treated with or without S961. (FIG. 10C) Expression of marker genes for inflammation and ER-stress at 2 weeks post S961 treatment. Expression levels of the markers in lysates of liver treated with or without S961 were detected by quantitative PCR method in the liver of PBS-treated (n=6) and S961-treated (n=6) mice. Data are shown as the mean±SEM.

FIGS. 11A-11N. Ago1-deficiency in the liver does not affect weight gain, liverweight, systemic energy expenditure, and glucose metabolism on HFD. (FIG. 11A) Body weights of L-Ago1 WT (n=8) and KO (n=8) mice fed HFD, starting at 4 weeks of age. (FIG. 11B) Glucose tolerance test of L-Ago1 WT (n=17) and L-Ago1 KO (n=15) mice fed HFD at 13 weeks of age. (FIG. 11C) Insulin tolerance test of L-Ago1 WT (n=17) and L-Ago1 KO (n=15) mice fed HFD at 14 weeks of age. (FIG. 11D) Pyruvate tolerance test of L-Ago1 WT (n=8) and L-Ago1 KO (n=8) mice fed HFD at 17 weeks of age. (FIG. 11E) Glucose tolerance test of L-Ago1 WT (n=16) and L-Ago1 KO (n=15) mice fed HFD at 20 weeks of age. (FIG. 11F) Insulin tolerance test of L-Ago1 WT (n=16) and L-Ago1 KO (n=15) mice fed HFD at 21 weeks of age. (FIGS. 11G-11I) Percent of liver weight per body weight (FIG. 11G), liver triglyceride content (FIG. 11H), and serum ALT levels (FIG. 11I) in L-Ago1 WT (n=8) and KO (n=8) mice fed HFD at 23 weeks of age. (FIGS. 11J and 11K) Fat (FIG. 11J) and lean mass (FIG. 11K) as a percentage of body weight in L-Ago1 WT (n=8) and KO (n=8) mice fed HFD at 20 weeks of age. (FIGS. 11L and 11M) Effects of a 14 hours fast on fat mass (FIG. 11L) and lean body mass (FIG. 11M) in L-Ago1 WT (n=7) and KO (n=7) mice fed HFD at 24 weeks of age. (FIG. 11N) Expression analyses of genes regulating energy metabolism in the liver of L-Ago1 WT (n=5) and L-Ago1 KO (n=5) fed HFD for 19 weeks.

FIGS. 12A-12M. Ago2-deficiency in the liver improves systemic glucose metabolism. (FIG. 12A) Glucose tolerance test performed in L-Ago2 WT (n=17) and KO (n=18) mice fed HFD at 13 weeks of age. (FIG. 12B) Insulin tolerance test of L-Ago2 WT (n=16) and KO (n=17) mice fed HFD at 21 weeks of age. (FIG. 12C) Enhanced insulin signaling, which was assessed by anti-phospho-specific Akt antibody, observed in the liver of L-Ago2 KO mice fed HFD at 30 weeks of age. (FIG. 12D) Expression levels of Tnfa in the liver of L-Ago2 WT (n=8) and L-Ago2 KO (n=5) mice fed HFD at 23 weeks of age. (FIG. 12E) Effects of hepatic Ago2-deletion on IRS1 inhibitory serine phosphorylation and JNK phosphorylation. Serine phosphorylation level of IRS1 was detected by an anti-phospho IRS1 antibody recognizing residue 307 and activation levels of JNK were detected by anti-phospho-JNK antibody in liver lysates of L-Ago2 WT and KO fed CD or HFD at 30 weeks of age. (FIG. 12F) Immunohistochemical staining of insulin (green), glucagon (white) and DAPI (blue) in paraffin section (5 μm) of mouse pancreas. (FIGS. 12G-12J) Mean islet size (FIG. 12G), quantification of percentage of insulin-stained area (FIG. 12H), glucagon-stained area (FIG. 12I) in full pancreas section area, and stained insulin to glucagon ratio (FIG. 12J), collected from L-Ago2 WT (n=7) and L-Ago2 KO (n=7) mice fed HFD and L-Ago2 WT (n=5) and L-Ago2 KO (n=4) mice fed CD at 33 weeks of age. (FIGS. 12K-12M) Hyperinsulinemic-euglycemic clamp studies performed in L-Ago2 WT (n=6) and L-Ago2 KO (n=10) mice fed HFD for 20 weeks. (FIG. 12K) blood glucose levels throughout the clamp procedures. The graph on the right shows averages of blood glucose levels. (FIG. 12L) Tissue glucose uptakes in brown fat (BAT) and heart. (FIG. 12M) Hepatic glucose production (HGP) during the clamp. The graphs show the quantification of the results. Data are shown as the mean±SEM. *p<0.05, **p<0.01.

FIGS. 13A-13L. Effects of liver-specific Ago2 deficiency on circulating parameters and energy metabolism. (FIGS. 13A-13D) Plasma triglyceride (FIG. 13A), cholesterol (FIG. 13B), phospolipids (FIG. 13C), and NEFA (FIG. 13D) levels in L-Ago2 WT (n=8) and L-Ago2 KO (n=6) mice fed HFD at 15 weeks of age. (FIGS. 13E and 13F) Fat (FIG. 13E) and lean mass (FIG. 13F) as a percentage of body weight of L-Ago2 WT (n=7) and L-Ago2 KO (n=7) mice fed HFD for 16 weeks. (FIGS. 13G-13K) VO2 (FIG. 13G), VCO2 (FIG. 13H), RER (FIG. 13I) Activity (FIG. 13J), and food intake (FIG. 13K) measured by TSE PhenoMaster system in L-Ago2 WT (n=8) and L-Ago2 KO (n=8) mice fed HFD for 8 weeks. (FIG. 13L) Fecal lipids excretion (13L) in L-Ago2 WT (n=8) and L-Ago2 KO (n=8) mice fed HFD for 14 weeks.

FIGS. 14A-14E. Identification of Ago2-dependent miRNAs and their target sequence in 3′ UTR of Ampka1 and Cs. (FIG. 14A) Expression levels of specific MD-miRNAs in the liver ob/ob mice (n=7) and their lean controls (n=5). (FIG. 14B) Venn diagram showing miRNAs reduced in the liver of L-Ago2 KO compared to L-Ago2 WT fed CD, fed HFD, and treated with S961. (FIG. 14C) Lists of miRNAs whose levels are significantly downregulated in the liver of Ago2 KO mice in lean, HFD-feeding, and S961 treated conditions. (FIGS. 14D and 14E) Potential binding sites of Ago2-dependent miRNAs in the 3′ UTR of Ampka1 (FIG. 14D) and Cs (FIG. 14E) showing species conservation.

FIGS. 15A-15C Enhanced activation of the AMPK pathway by hepatic Ago2 deficiency. (FIG. 15A) Western blot analyses of the liver lysates from L-Ago2 WT and KO mice fed HFD for 21 weeks. (FIG. 15B) Western blot analyses of the AMPK pathway in the liver of L-Ago2 WT and L-Ago2 KO treated with S961 for 2 weeks. (FIG. 15C) Western blot analysis of total and specific protein levels normalized by 12S-genomic DNA in the liver of L-Ago2 WT (n=5) and KO (n=5) mice fed HFD for 26 weeks. The graphs show the quantification of the results. Data are shown as the mean±SEM. *p<0.05, “p<0.01.

FIGS. 16A-16C. Ago2-deficiency in the liver enhances energy expenditure linked AMPK activation and protein translation in MEFs. (FIGS. 16A and 16B) Effect of Ago2 WT or slicer activity-defect Ago2 mutant (DA) reconstitution on expression of Ampka1 mRNA and AMPK protein, and activation of AMPK (FIG. 16A), and nascent protein synthesis (FIG. 16B) in Ago2-deficient MEFs. (FIG. 16C) AMPK activation in Ago2 WT and KO MEFs in the presence or absence of 25 μg/ml cycloheximide (CHX) for 8 hours.

FIGS. 17A-17H. Uncropped Western blots. (FIGS. 17A-17H) Dot line boxes indicate the cropped areas shown in the figures.

### DETAILED DESCRIPTION

Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, or up to 10%, or up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein the language “pharmaceutically acceptable carrier” is intended to include any and all solvents, dispersion media, coatings, antibacterial and antifungal agents, isotonic and absorption delaying agents, and the like, compatible with pharmaceutical administration. Pharmaceutically acceptable carriers include a wide range of known diluents (i.e., solvents), fillers, extending agents, binders, suspending agents, disintegrates, surfactants, lubricants, excipients, wetting agents and the like commonly used in this field. These carriers may be used singly or in combination according to the form of the pharmaceutical preparation, and may further encompass “pharmaceutically acceptable excipients” as defined herein.

As used herein, “pharmaceutically acceptable excipient” means any other component added to a pharmaceutical formulation other than the active ingredient and which is capable of bulking-up formulations that contain potent active ingredients (thus often referred to as “bulking agents,” “fillers,” or “diluents”) to allow convenient and accurate dispensation of a drug substance when producing a dosage form. Excipients may be added to facilitate manufacture, enhance stability, control release, enhance product characteristics, enhance bioavailability drug absorption or solubility, or other pharmacokinetic considerations, enhance patient acceptability, etc. Pharmaceutical excipients include, for example, carriers, fillers, binders, disintegrants, lubricants, glidants, colors, preservatives, suspending agents, dispersing agents, film formers, buffer agents, pH adjusters, preservatives etc. The selection of appropriate excipients also depends upon the route of administration and the dosage form, as well as the active ingredient and other factors, and will be readily understood by one of ordinary skill in the art.

As used herein, the term “therapeutically effective amount” means the total amount of each active component of the pharmaceutical composition or method that is sufficient to show a meaningful patient benefit, e.g., healing of chronic conditions or in an increase in rate of healing of such conditions, or in a reduction in aberrant conditions. This includes both therapeutic and prophylactic treatments. Accordingly, the compounds can be used at very early stages of a disease, or before early onset, or after significant progression. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

The active agent can form salts, which are also within the scope of the preferred embodiments. Reference to a compound of the active agent herein is understood to include reference to salts thereof, unless otherwise indicated. The term “salt(s)”, as employed herein, denotes acidic and/or basic salts formed with inorganic and/or organic acids and bases. In addition, when an active agent contains both a basic moiety, such as, but not limited to an amine or a pyridine or imidazole ring, and an acidic moiety, such as, but not limited to a carboxylic acid, zwitterions (“inner salts”) can be formed and are included within the term “salt(s)” as used herein. Pharmaceutically acceptable (e.g., non-toxic, physiologically acceptable) salts are preferred, although other salts are also useful, e.g., in isolation or purification steps, which can be employed during preparation. Salts of the compounds of the active agent can be formed, for example, by reacting a compound of the active agent with an amount of acid or base, such as an equivalent amount, in a medium such as one in which the salt precipitates or in an aqueous medium followed by lyophilization.

When the compounds are in the forms of salts, they may comprise pharmaceutically acceptable salts. Such salts may include pharmaceutically acceptable acid addition salts, pharmaceutically acceptable base addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like.

The worldwide prevalence of obesity has reached pandemic proportions, bringing with it a host of associated diseases, such as type 2 diabetes (T2D), non-alcoholic steatohepatitis (NASH), and cancer^{1,2}. Obesity develops when energy intake chronically exceeds total energy expenditure. Basal metabolic rate represents the largest component of total energy expenditure, of which the liver is a major organ for energy consumption3. Protein biosynthesis is one of the most energy consuming cellular processes in the liver, accounting for approximately 20-30% of total energy consumption^{4,5}. However, despite abundant supply of energy sources and a robust activation of the mammalian target of rapamycin complex (mTORC) pathway, a main driver of protein synthesis⁶⁻⁹, the liver-driven energy consumption robustly declined in obesity due to, at least in part, insufficient protein biosynthesis. Suppression of hepatic protein synthesis leads to further accumulation of energy sources associated with obesity-associated pathophysiology, however, the exact mechanism(s) of insufficient protein biosynthesis remains unclear. Hence, defining such molecular mechanism(s) could provide a novel therapeutic approach that alters energy balance in obesity and modulates the pathogenesis of associated sequelae.

Disclosed herein are methods of treating a metabolic disorder in an individual in need thereof. The methods may comprise the step of administering a therapeutically effective amount of an agent that interferes with Ago2 activity and/or function in combination with a pharmaceutically acceptable excipient. In one aspect, the metabolic disorder may be selected from obesity, type II diabetes, heart disease, liver disease, or combinations thereof. In one aspect, the metabolic disorder is fatty liver disease. In one aspect, the agent that interferes with Ago2 activity and/or function is selected from an inhibitory antibody specific for Ago2, an inhibitory nucleotide specific for Ago2, or a combination thereof. Synthesis of the foregoing will be readily appreciated by one of ordinary skill in the art.

In one aspect, the agent that interferes with Ago2 activity and/or function may be selected from trypaflavine (TPF)

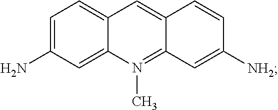

described in Watashi et al, “Identification of Small Molecules that Suppress MicroRNA Function and Reverse Tumorigenesis,” JBC Vo. 285, pp 24707-24716 (2010);

Bcl-137 or 2-(2,3-Dioxo-1,2,3,4-tetrahydroquinoxaline-6-sulfonamido) propanoic acid

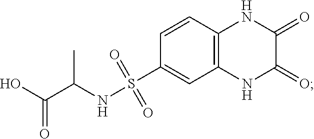 [SMILES: CC(NS(=O)(=O)c1ccc2[nH]c(=O)c(=O)[nH]c2c1)C(=O)O]

available from Millipore Sigma,

aurintricarboxylic acid (ACF)

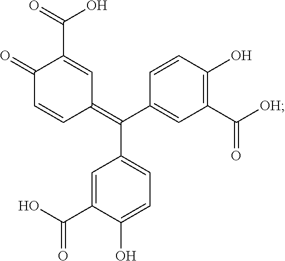 [SMILES: O=C(O)C1=CC(=C(c2ccc(O)c(C(=O)O)c2)c2ccc(O)c(C(=O)O)c2)C=CC1=O]

suramin

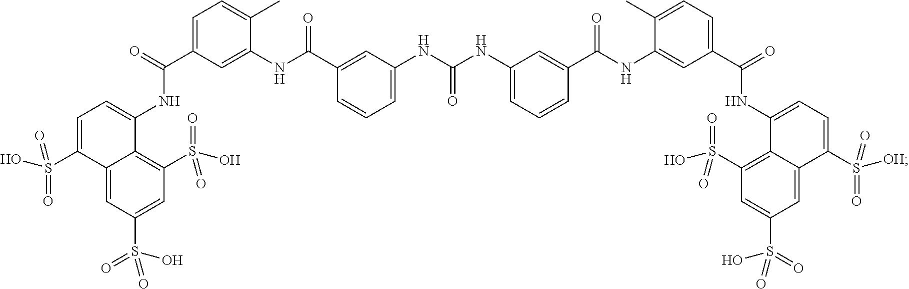 [SMILES: Cc1ccc(C(=O)Nc2ccc(S(=O)(=O)O)c3cc(S(=O)(=O)O)cc(S(=O)(=O)O)c23)cc1NC(=O)c1cccc(NC(=O)Nc2cccc(C(=O)Nc3cc(C(=O)Nc4ccc(S(=O)(=O)O)c5cc(S(=O)(=O)O)cc(S(=O)(=O)O)c45)ccc3C)c2)c1]

oxidopamine HCL

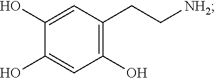 [SMILES: NCCc1cc(O)c(O)cc1O]

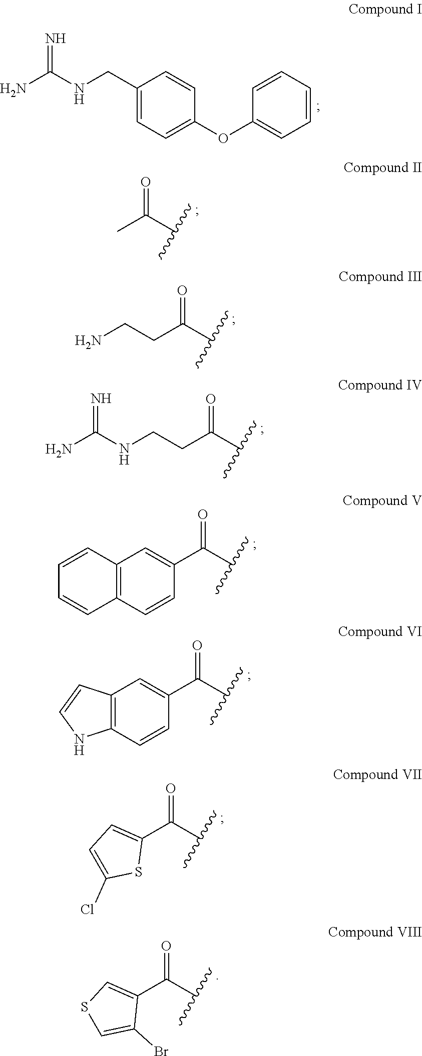

Compounds I-VIII are described in Schmidt et al, MicroRNA-Specific Argonaute 2 Protein Inhibitors, ACS Chem. Biol. 2013, 8, 2122-2126.

The disclosed compounds include pharmaceutically acceptable salts thereof and the compositions may contain combinations of any of the foregoing.

In one aspect, the agent may be administered via a route selected from orally, topically, parenterally, by inhalation or spray, vaginally, rectally, sublingually in dosage unit formulations, or a combination thereof.

In one aspect, a method of improving systemic glucose metabolism in the liver on an individual in need thereof is disclosed. In this aspect, the method may comprise the step of administering a therapeutically effective amount of an agent that interferes with Ago2 activity and/or function in combination with a pharmaceutically acceptable excipient. The method may employ any of the above-disclosed compounds, and any combination or salt thereof.

In one aspect, a therapeutic kit is disclosed. The kit may comprise a) a composition as disclosed above, and b) a means for delivery of the composition to a human.

In one aspect, disclosed is an article of manufacture that may comprise a) a container comprising a label; and b) a composition as disclosed above, wherein the label indicates that the composition is to be administered to an individual in need of treatment for a systemic glucose metabolism related condition.

The active compounds and/or pharmaceutical compositions of the embodiments disclosed herein can be administered according to various routes. The compounds can be administered orally, topically, parenterally, by inhalation or spray, vaginally, rectally or sublingually in dosage unit formulations. The term “administration by injection” includes but is not limited to: intravenous, intraarticular, intramuscular, subcutaneous and parenteral injections, as well as use of infusion techniques. Dermal administration can include topical application or transdermal administration. Furthermore, repeated injections can be performed, if needed, although it is believed that limited injections will be needed in view of the efficacy of the compounds.

The compounds may also be used enterally. Orally, the compounds may be administered at the rate of 100 μg to 100 mg per day per kg of body weight. Orally, the compounds may be suitably administered at the rate of about 100, 150, 200, 250, 300, 350, 400, 450, or 500 μg to about 1, 5, 10, 25, 50, 75, 100 mg per day per kg of body weight. The required dose can be administered in one or more portions. For oral administration, suitable forms are, for example, tablets, gel, aerosols, pills, dragees, syrups, suspensions, emulsions, solutions, powders and granules; one method of administration includes using a suitable form containing from 1 mg to about 500 mg of active substance. In one aspect, administration may comprise using a suitable form containing from about 1, 2, 5, 10, 25, or 50 mg to about 100, 200, 300, 400, 500 mg of active substance.

The compounds may also be administered parenterally in the form of solutions or suspensions for intravenous or intramuscular perfusions or injections. In that case, the compounds may be administered at the rate of about 10 μg to 10 mg per day per kg of body weight; one method of administration may consist of using solutions or suspensions containing approximately from 0.01 mg to 1 mg of active substance per ml. The compounds may be administered at the rate of about 10, 20, 30, 40, 50, 60, 70, 80, 90, or 100 μg to 1, 2, 3, 4, 5, 6, 7, 8, 9, or 10 mg per day per kg of body weight; in one aspect, solutions or suspensions containing approximately from 0.01, 0.02, 0.03, 0.04, or 0.5 mg to 0.1, 0.2, 0.3, 0.4, 0.5, 0.6, 0.7, 0.8, 0.9, or 1 mg of active substance per ml may be used.

The form and administration route for the pharmaceutical composition are not limited and can be suitably selected. For example, tablets, capsules, granules, pills, syrups, solutions, emulsions, and suspensions may be administered orally. Additionally, injections (e.g. subcutaneous, intravenous, intramuscular, and intraperitoneal) may be administered intravenously either singly or in combination with a conventional replenisher containing glucose, amino acid and/or the like, or may be singly administered intramuscularly, intracutaneously, subcutaneously and/or intraperitoneally.

Compounds may also be administrated transdermally using methods known to those skilled in the art. For example, a solution or suspension of an active agent in a suitable volatile solvent optionally containing penetration enhancing agents can be combined with additional additives known to those skilled in the art, such as matrix materials and bacteriocides. After sterilization, the resulting mixture can be formulated following known procedures into dosage forms. In addition, on treatment with emulsifying agents and water, a solution or suspension of an active agent can be formulated into a lotion or salve.

The compounds can also be administered in the form of suppositories for rectal or vaginal administration of the drug. These compositions can be prepared by mixing the drug with a suitable nonirritating excipient which is solid at ordinary temperatures but liquid at the rectal temperature or vaginal temperature and will therefore melt in the rectum or vagina to release the drug. Such materials include cocoa butter and polyethylene glycols.

It will be appreciated by those skilled in the art that the particular method of administration will depend on a variety of factors, all of which are considered routinely when administering therapeutics. It will also be understood, however, that the specific dose level for any given patient will depend upon a variety of factors, including, the activity of the specific compound employed, the age of the patient, the body weight of the patient, the general health of the patient, the gender of the patient, the diet of the patient, time of administration, route of administration, rate of excretion, drug combinations, and the severity of the condition undergoing therapy. It will be further appreciated by one skilled in the art that the optimal course of treatment, i.e., the mode of treatment and the daily number of doses of an active agent or a pharmaceutically acceptable salt thereof given for a defined number of days, can be ascertained by those skilled in the art using conventional treatment tests.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

RNA silencing is an inhibitory process of mRNA translation. While mRNA translation accounts for the majority of cellular energy expenditure, it is unclear if RNA silencing regulates energy homeostasis. Here, Applicant has found that hepatic Argonaute 2 (Ago2)-mediated RNA silencing intrinsically functions to suppress both energy production and consumption, thereby disturbing energy metabolism in the pathogenesis of obesity. Ago2 regulates biogenesis of specific miRNAs including miR-802, miR-103/107, and miR-148a/152, causing metabolic disruption, while simultaneously suppressing expression of genes regulating glucose and lipid metabolism, including Hnf1β, Cav1, and Ampka1. Liver-specific deletion of Ago2 enhances mitochondrial oxidation and ATP consumption associated with mRNA translation, which results in AMPK activation, and improves obesity-associated pathophysiology. Notably, hepatic Ago2-deficiency blunts the effects of Ampka1 -deletion in liver and treatment with an anti-diabetic drug metformin in improving glucose metabolism. The regulation of energy metabolism by Ago2 provides a novel paradigm in which RNA silencing plays an integral role in determining basal metabolic activity in obesity-associated sequelae.

Recent studies have revealed significant roles for microRNA (miRNA)-mediated events in the development and progression of obesity and its associated sequelae^{10,11}. Of note, global dysregulation of miRNA biogenesis is triggered in the human and murine obese liver, leading to the induction of the vast majority of miRNAs, including miR-802, miR-103/miR-107, and miR-148a that deteriorate glucose and lipid metabolism in obesity¹²⁻¹⁶. As miRNA generally inhibits the translation of target mRNAs through RNA silencing, it is reasonable to hypothesize that these induced miRNAs may contribute to suppression of protein biosynthesis and its associated energy expenditure in obese liver. However, there remain fundamental questions concerning why and how these miRNAs are concurrently induced in the obese condition and whether RNA silencing is integrated into an elaborate adaptive program that cells can elicit to balance anabolic and catabolic processes dependent on energy and metabolic statuses. If RNA silencing plays a role in protein biosynthesis-associated energy metabolism, one would anticipate that individual component(s) of miRNA-regulatory machinery in the liver may impinge on metabolic regulation, and that a nutrient challenge might accentuate the consequences of this regulation.

Argonaute (Ago) family proteins are the main components of the RNA-induced silencing complex (RISC) that carries out RNA silencing. Upon loading of Ago proteins with mature miRNAs produced by the endoribonuclease Dicer^{17,18}, RISC represses the expression of targeted mRNA through RNA silencing^{18-20.} Amongst all Ago proteins, Ago2 specifically possesses an endoribonuclease (“slicer”) activity that generates a specific mature miRNA and cleaves targeted mRNAs in mammals^{18,21-24.} To study the role of RNA silencing in hepatic energy homeostasis, Applicant comprehensively evaluated the role of hepatic Ago1 and Ago2, as analyses of these core RISC components might lead to fundamental insights into the link of RNA silencing with energy metabolism. Applicant has demonstrated that hepatic Ago2-mediated RNA silencing suppresses energy expenditure and its inactivation protects from obesity-associated glucose intolerance and hepatic steatosis in mice. More importantly, Applicant discover novel roles of Ago2 in orchestrating the expression of a set of miRNAs, including miR-802, miR-103/107, and miR-148a, and in the regulation of AMP-activated protein kinase (AMPK) activation linked to protein biosynthesis-mediated energy consumption, for which Ago2's slicer activity critically functions. This Ago2-mediated RNA silencing is a core mechanism that connects the dots between protein translation, energy production and consumption, and AMPK activity—disruption of such events is a well-recognized feature in obesity and the pathogenesis of obesity-associated sequelae.

Hepatic Ago2 Regulates Expression of Specific miRNAs Involved in Energy Metabolism

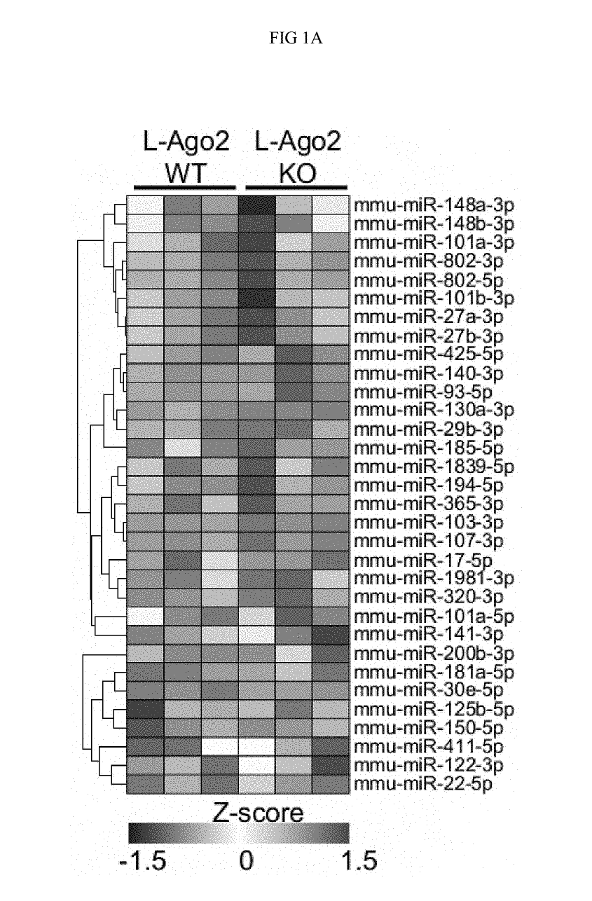

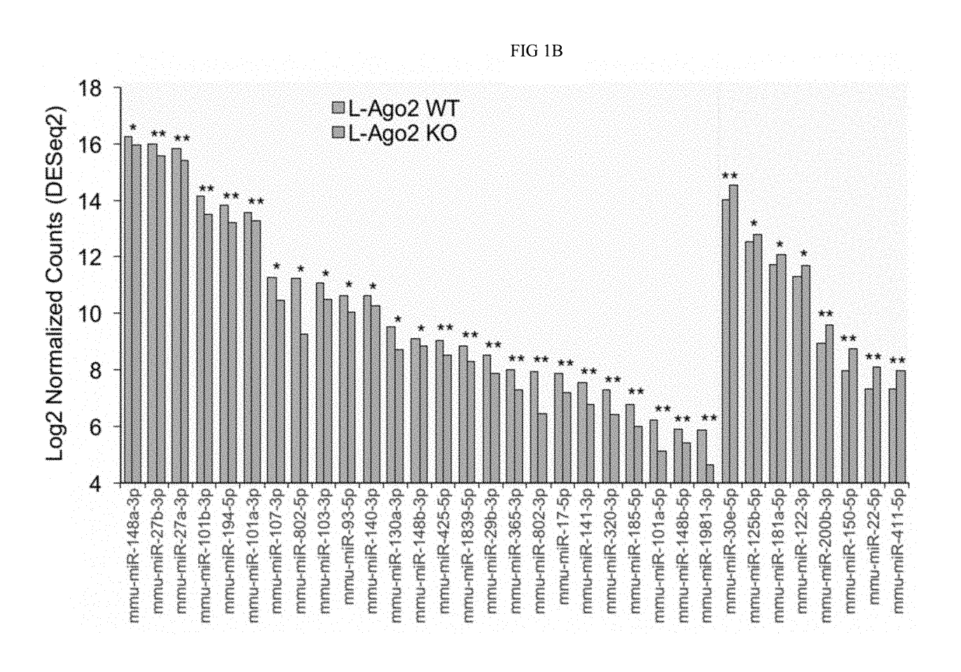

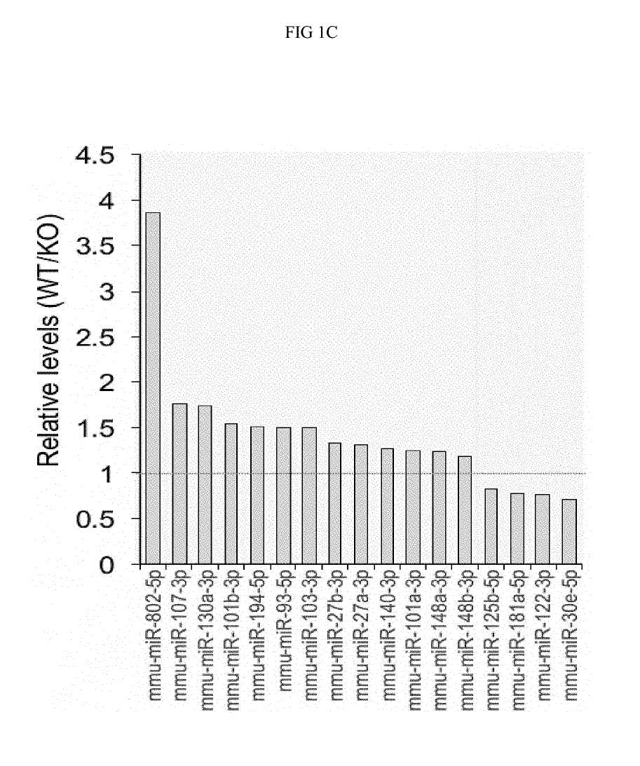

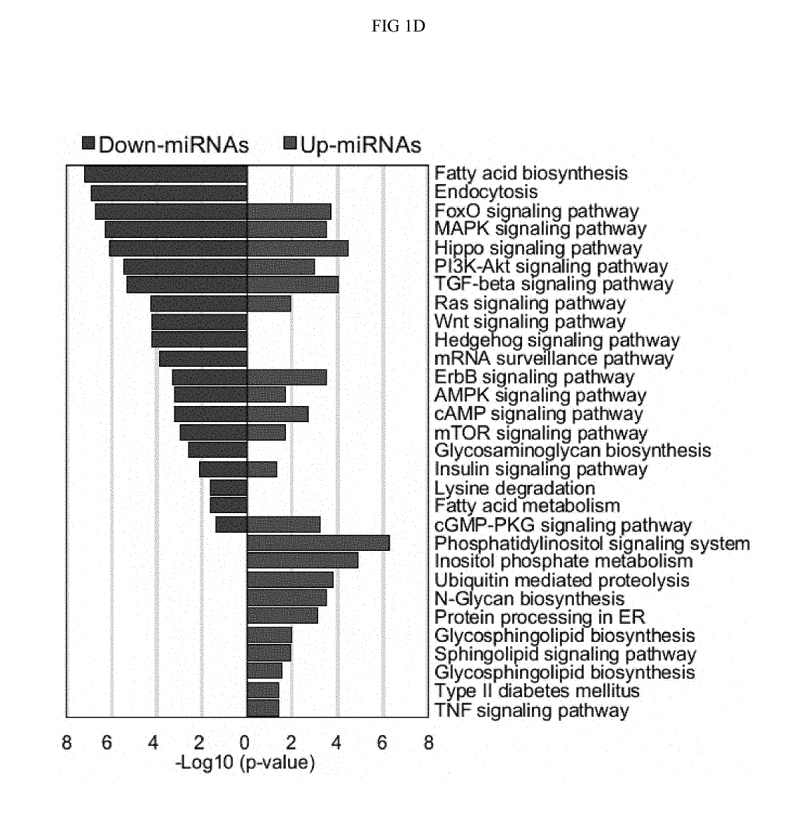

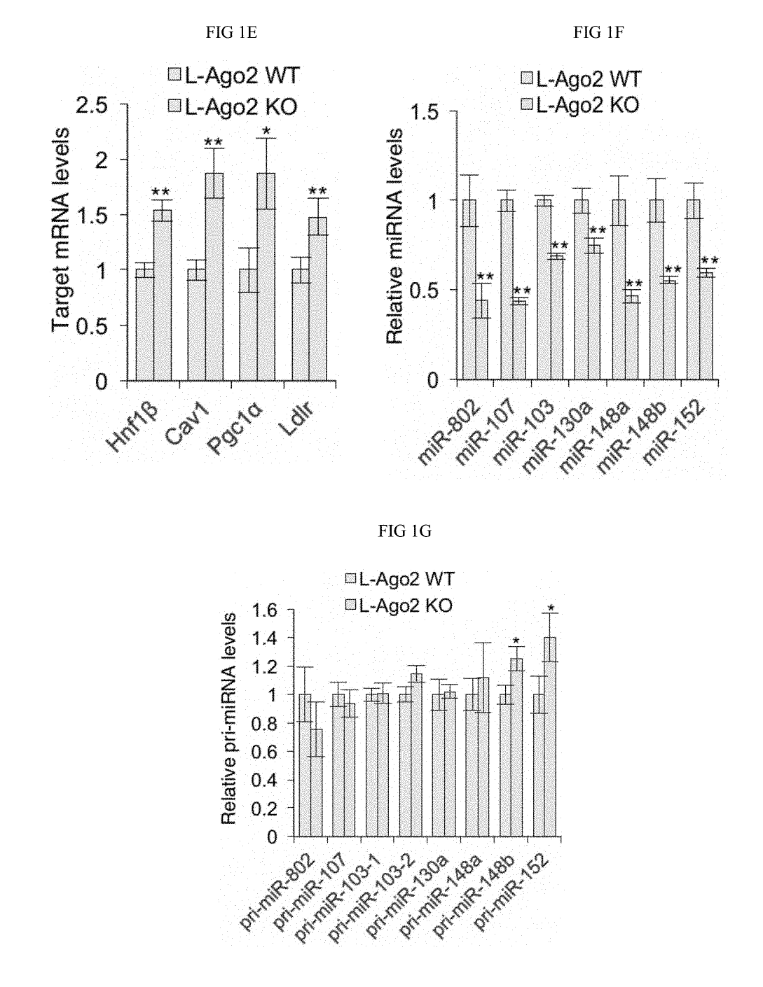

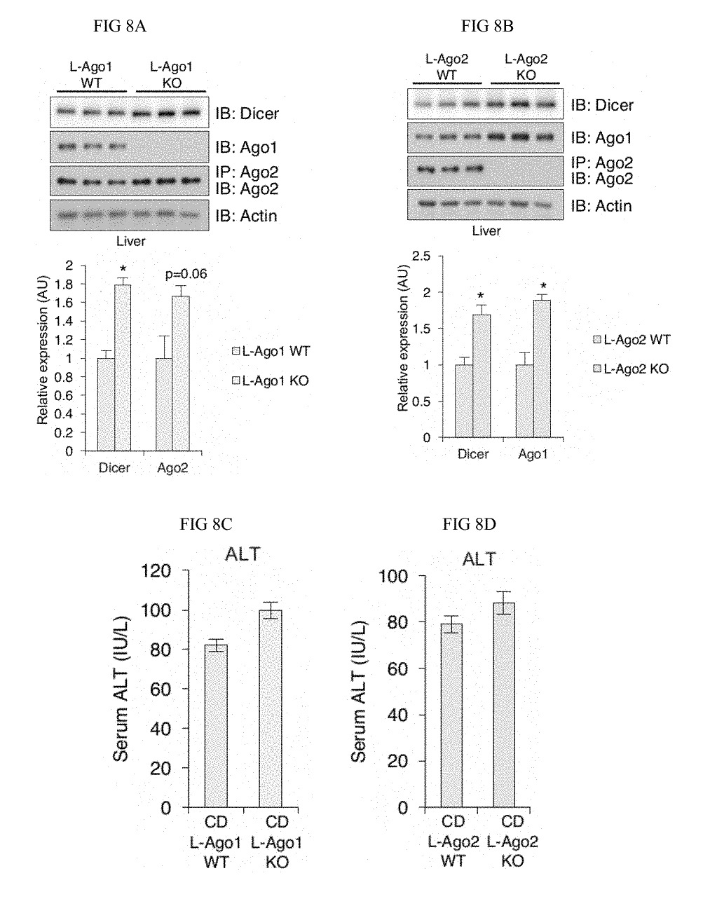

Ago1 and Ago2 are the predominant Ago family members expressed in the liver25. To investigate if RNA silencing is associated with energy and metabolic homeostasis, Applicant generated liver-specific Ago1-deficient (L-Ago1 KO: Ago1^{fl/fl} Alb-Cre^{Tg/0}) and Ago2-deficient (L-Ago2 KO: Ago1^{fl/fl} Alb-Cre^{Tg/0}) mice. Deletion of Ago1 or Ago2 in the liver was confirmed by western blot analysis (FIG. 8a and 1b). On control diet (CD), both L-Ago1 KO and L-78 Ago2 KO mice gained weight similarly to their controls, L-Ago1 WT (Ago1 ^{fl/fl} Alb-Cre^{0/0}) or L-79 Ago2 WT (Ago21^{fl/fl} Alb-Cre^{0/0}), and showed no obvious abnormalities during development or in adulthood. In addition, levels of serum alanine aminotransferase (ALT) were comparable between the groups (FIGS. 8c and 8d). These results suggest that general functions of the liver are likely unaffected by the absence of Ago1 or Ago2 during development in regular feeding conditions.

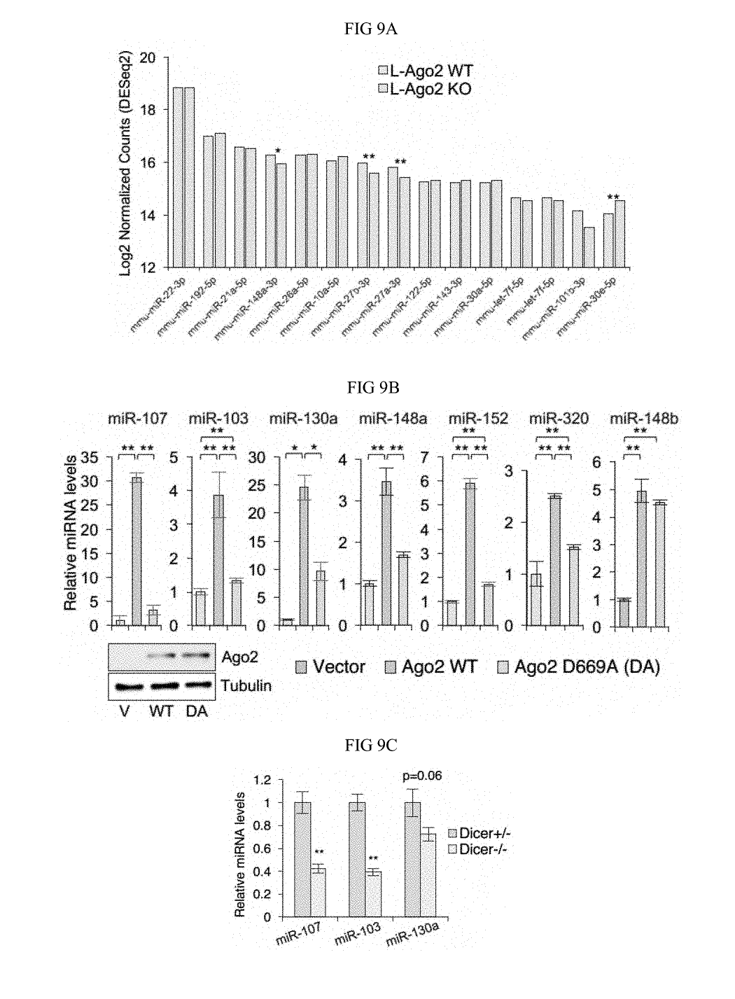

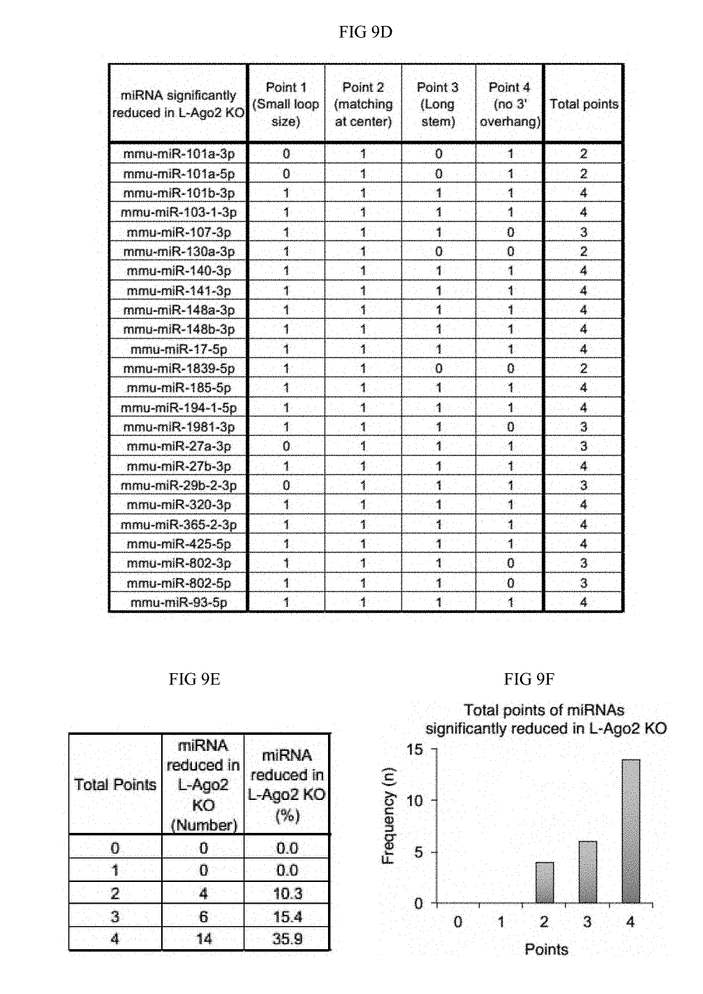

Among the Ago proteins, Ago2 uniquely possesses a slicer activity known to contribute to the biogenesis of specific miRNA²¹⁻²³ and mRNA cleavage^{18,26}. In the Ago2-deficient liver, expression levels of Ago1 are increased (FIG. 8B), and therefore Ago1 may compensate for Ago2's non-slicer activity-dependent function. To gain insight into the specific roles of Ago2's activity in the regulation of liver function, Applicant first assessed the effect of hepatic Ago2-deficiency on miRNA expression profile by performing next-generation sequencing for miRNA. Expression profile analyses revealed that the expression levels of 25 miRNAs were significantly reduced in L-Ago2 KO liver, while 8 miRNAs were significantly increased (FIGS. 8A and 8B). Among these significant miRNAs, miR-148a is one of the most abundant miRNAs expressing in the WT liver (FIG. 1B and FIG. 9A). In addition, 17 out of 33 total significant miRNAs abundantly expressed were in the top 10 percent of miRNAs expressing in the WT liver (FIG. 1C and Table 1). Intriguingly, this group contained miRNAs known to be associated with metabolic diseases (MD-miRNAs) and detrimental to glucose and lipid metabolism, including miR-802, miR-103/107, miR-130a, and miR-148a^{12,13,27-29}, which were down-regulated in the L-Ago2 KO liver (FIG. 1C). Applicant additionally utilized the list of significant miRNAs altered in Ago2 KO liver through the miRNA enrichment pathway analysis by analyzing the biological processes and molecular function categories (FIG. 1D). Hepatic Ago2 deficiency affected the functional clades associated with energy metabolism including fatty acid biosynthesis, AMP-activated protein kinase (AMPK) signaling, protein processing, and insulin signaling. Taken together, these results imply the unique role of Ago2 in the expressional regulation of a specific repertoire of MD-miRNA for energy metabolism and suggest the possibility that Ago2 may have specialized roles for metabolic regulations. Consistent with these observations, the reduction of MD-miRNAs in L-Ago2 KO liver is accompanied by increased expression of their known target mRNAs, such as hepatocyte nuclear factor 1 homeobox B (Hnf1β), caveolin-1 (Cav1), peroxisome proliferator-activated receptor gamma coactivator 1α (Pgc1α), and low density lipoprotein receptor (Ldlr), which regulates energy metabolism (FIG. 1E)^{12,13,29}.

TABLE 1
Analysis of miRNA expression profile in the liver of L-Ago2 WT
and KO mice.

| | | | Relative Levels |
|---|---|---|---|
| | L-Ago2 WT | L-Ago2 KO | (WT/KO) |
| mmu-mir-22, mmu-miR-22-3p | 474036.5679 | 473406.1337 | 1.001331698 |
| mmu-mir-192, mmu-miR-192-5p | 129635.1417 | 141111.9517 | 0.91866876 |
| mmu-mir-21a, mmu-miR-21a-5p | 97265.77427 | 93993.27762 | 1.034816284 |
| mmu-mir-148a, mmu-miR-148a-3p | 78595.54939 | 63169.6409 | 1.244198135 |
| mmu-mir-26a-2, mmu-miR-26a-5p | 78438.26342 | 81160.00777 | 0.966464464 |
| mmu-mir-10a, mmu-miR-10a-5p | 68232.23662 | 75835.38845 | 0.899741374 |
| mmu-mir-27b, mmu-miR-27b-3p | 64632.15311 | 48683.47854 | 1.327599322 |
| mmu-mir-27a, mmu-miR-27a-3p | 57994.79684 | 44232.24905 | 1.311142844 |
| mmu-mir-122, mmu-miR-122-5p | 38882.29391 | 39958.87866 | 0.973057683 |
| mmu-mir-143, mmu-miR-143-3p | 38097.75955 | 40414.97379 | 0.942664463 |
| mmu-mir-30a, mmu-miR-30a-5p | 38039.06879 | 41287.54381 | 0.921320701 |
| mmu-let-7f-1, mmu-let-7f-5p | 25461.72401 | 23591.93667 | 1.079255356 |
| mmu-mir-191, mmu-miR-191-5p | 18555.21117 | 16661.85803 | 1.113633974 |
| mmu-mir-101b, mmu-miR-101b-3p | 18008.58708 | 11683.03535 | 1.541430506 |
| mmu-mir-30e, mmu-miR-30e-5p | 16951.09138 | 23618.12037 | 0.717715513 |
| mmu-mir-92a-1, mmu-miR-92a-3p | 14996.62208 | 16532.51725 | 0.907098529 |
| mmu-mir-194-1, mmu-miR-194-5p | 14531.69849 | 9629.031272 | 1.509154771 |
| mmu-mir-26b, mmu-miR-26b-5p | 14250.8989 | 14264.58975 | 0.999040221 |
| mmu-mir-30c-1, mmu-miR-30c-5p | 14163.65048 | 15126.19512 | 0.936365714 |
| mmu-mir-99b, mmu-miR-99b-5p | 14058.27702 | 13852.75595 | 1.014836114 |
| mmu-mir-29a, mmu-miR-29a-3p | 12691.95895 | 12188.29459 | 1.041323613 |
| mmu-mir-101a, mmu-miR-101a-3p | 12241.94368 | 9802.275449 | 1.248887949 |
| mmu-mir-92a-2, mmu-miR-92a-3p | 12115.07217 | 14074.33941 | 0.860791531 |
| mmu-mir-378a, mmu-miR-378a-3p | 11223.01157 | 10844.70422 | 1.034884063 |
| mmu-mir-125a, mmu-miR-125a-5p | 10536.76946 | 10267.22245 | 1.026253157 |
| mmu-let-7a-1, mmu-let-7a-5p | 9438.46214 | 9124.685797 | 1.034387633 |
| mmu-mir-100, mmu-miR-100-5p | 8305.586844 | 9741.867417 | 0.852566196 |
| mmu-mir-30d, mmu-miR-30d-5p | 8199.385633 | 8145.013188 | 1.00667555 |
| mmu-let-7g, mmu-let-7g-5p | 8174.055739 | 7226.942108 | 1.131053164 |
| mmu-mir-378c, mmu-miR-378c | 6885.994957 | 6042.082268 | 1.139672492 |
| mmu-mir-486a, mmu-miR-486a-5p | 6570.978009 | 7344.09133 | 0.894729887 |
| mmu-mir-486b, mmu-miR-486b-5p | 6564.717711 | 7334.029687 | 0.895103782 |
| mmu-mir-125b-1, mmu-miR-125b-5p | 5977.867157 | 7248.404571 | 0.824714887 |
| mmu-mir-151, mmu-miR-151-5p | 5461.492675 | 5520.729226 | 0.989270158 |
| mmu-mir-126a, mmu-miR-126a-3p | 4998.664684 | 5809.684125 | 0.860402145 |
| mmu-mir-30b, mmu-miR-30b-5p | 4859.886395 | 4879.057619 | 0.996070712 |
| mmu-let-7c-2, mmu-let-7c-5p | 4655.984959 | 4685.04095 | 0.993798135 |
| mmu-let-7c-1, mmu-let-7c-5p | 4652.854701 | 4677.91073 | 0.994643757 |
| mmu-mir-142a, mmu-miR-142a-5p | 4275.795821 | 5352.665496 | 0.798816183 |
| mmu-mir-99a, mmu-miR-99a-5p | 3812.109952 | 3964.84009 | 0.961478866 |
| mmu-mir-181a-1, mmu-miR-181a-5p | 3423.459798 | 4359.551939 | 0.785277902 |
| mmu-mir-151, mmu-miR-151-3p | 3229.256872 | 3010.901942 | 1.072521435 |
| mmu-mir-122, mmu-miR-122-3p | 2566.777267 | 3335.200969 | 0.769601979 |
| mmu-mir-107, mmu-miR-107-3p | 2494.46579 | 1415.239536 | 1.762574976 |
| mmu-mir-802, mmu-miR-802-5p | 2399.389766 | 619.6246887 | 3.872327572 |
| mmu-let-7i, mmu-let-7i-5p | 2257.186113 | 2540.178047 | 0.888593662 |
| mmu-let-7d, mmu-let-7d-5p | 2243.348989 | 1940.969848 | 1.155787655 |
| mmu-mir-103-1, mmu-miR-103-3p | 2159.015525 | 1442.081419 | 1.497152308 |
| mmu-mir-29c, mmu-miR-29c-3p | 2111.767809 | 2287.713246 | 0.923091132 |
| mmu-mir-203, mmu-miR-203-3p | 2030.121628 | 1923.565571 | 1.055395074 |
| mmu-mir-451a, mmu-miR-451a | 2014.011122 | 2319.081238 | 0.868452165 |
| mmu-mir-15a, mmu-miR-15a-5p | 1611.519102 | 1916.634816 | 0.840806547 |
| mmu-mir-199a-1, mmu-miR-199a-3p | 1604.090444 | 1977.375903 | 0.811221802 |
| mmu-mir-199b, mmu-miR-199b-3p | 1604.090444 | 1977.375903 | 0.811221802 |
| mmu-mir-93, mmu-miR-93-5p | 1590.952897 | 1058.808363 | 1.502588148 |
| mmu-mir-186, mmu-miR-186-5p | 1578.965377 | 1320.10545 | 1.196090341 |
| mmu-mir-140, mmu-miR-140-3p | 1567.27108 | 1242.150041 | 1.261740554 |
| mmu-mir-25, mmu-miR-25-3p | 1555.206398 | 1573.332423 | 0.988479214 |
| mmu-mir-340, mmu-miR-340-5p | 1415.795299 | 1155.592951 | 1.225167822 |
| mmu-mir-10b, mmu-miR-10b-5p | 1400.13092 | 1385.240839 | 1.010749092 |
| mmu-mir-193a, mmu-miR-193a-3p | 1334.923053 | 1122.359081 | 1.189390344 |
| mmu-mir-21a, mmu-miR-21a-3p | 1174.560513 | 992.4980556 | 1.183438604 |
| mmu-let-7b, mmu-let-7b-5p | 1124.859631 | 1166.526038 | 0.964281632 |
| mmu-mir-31, mmu-miR-31-5p | 998.0168056 | 977.2368818 | 1.021263958 |
| mmu-mir-181c, mmu-miR-181c-5p | 858.9529755 | 874.616372 | 0.982091124 |
| mmu-mir-423, mmu-miR-423-3p | 848.9836023 | 820.5495387 | 1.034652464 |
| mmu-mir-145a, mmu-miR-145a-3p | 843.3584617 | 832.6542849 | 1.012855488 |
| mmu-mir-23b, mmu-miR-23b-3p | 804.3240749 | 722.047716 | 1.113948645 |
| mmu-mir-16-1, mmu-miR-16-5p | 799.047909 | 900.8268341 | 0.887016104 |
| mmu-mir-351, mmu-miR-351-5p | 781.102157 | 948.2732932 | 0.823709961 |
| mmu-mir-126a, mmu-miR-126a-5p | 748.1681075 | 871.255855 | 0.858723764 |
| mmu-mir-130a, mmu-miR-130a-3p | 722.5122801 | 416.4514339 | 1.734925663 |
| mmu-mir-148a, mmu-miR-148a-5p | 707.1439187 | 794.4648321 | 0.890088384 |
| mmu-mir-199a-1, mmu-miR-199a-5p | 699.5854413 | 855.6804444 | 0.817577924 |
| mmu-mir-1948, mmu-miR-1948-3p | 595.8536952 | 629.2797266 | 0.946882078 |
| mmu-mir-148b, mmu-miR-148b-3p | 552.4399224 | 463.3050087 | 1.192389273 |
| mmu-mir-322, mmu-miR-322-5p | 534.0884857 | 560.536907 | 0.952815914 |
| mmu-mir-425, mmu-miR-425-5p | 533.6559556 | 363.4629036 | 1.468254257 |
| mmu-mir-19b-1, mmu-miR-19b-3p | 526.4029598 | 550.6067525 | 0.956041599 |
| mmu-mir-1843a, mmu-miR-1843a-5p | 522.6939593 | 444.3091336 | 1.176419569 |
| mmu-mir-152, mmu-miR-152-3p | 514.3601609 | 392.7480062 | 1.309644232 |
| mmu-mir-200b, mmu-miR-200b-3p | 492.3698629 | 773.8618144 | 0.636250366 |
| mmu-mir-423, mmu-miR-423-5p | 463.0762441 | 409.8138098 | 1.129967397 |
| mmu-mir-1839, mmu-miR-1839-5p | 461.4433452 | 315.2518564 | 1.463729192 |
| mmu-mir-24-1, mmu-miR-24-3p | 458.7903479 | 466.7211194 | 0.983007472 |
| mmu-mir-24-2, mmu-miR-24-3p | 458.7903479 | 466.7211194 | 0.983007472 |
| mmu-mir-30a, mmu-miR-30a-3p | 435.6409928 | 365.2459515 | 1.192733255 |
| mmu-let-7d, mmu-let-7d-3p | 435.0454082 | 409.2401586 | 1.063056494 |
| mmu-mir-497a, mmu-miR-497a-5p | 420.3902858 | 485.6119355 | 0.865691831 |
| mmu-mir-98, mmu-miR-98-5p | 378.9867304 | 339.006276 | 1.11793426 |
| mmu-mir-501, mmu-miR-501-3p | 377.4181186 | 346.2074791 | 1.090150102 |
| mmu-mir-28a, mmu-miR-28a-5p | 369.8826042 | 310.077243 | 1.192872462 |
| mmu-mir-29b-1, mmu-miR-29b-3p | 365.5471132 | 233.5968626 | 1.564863111 |
| mmu-mir-335, mmu-miR-335-5p | 341.9413915 | 369.8869297 | 0.92444843 |
| mmu-mir-532, mmu-miR-532-5p | 340.6996865 | 282.1620408 | 1.207461094 |
| mmu-let-7e, mmu-let-7e-5p | 300.8311071 | 273.112732 | 1.1014906 |
| mmu-mir-1843b, mmu-miR-1843b-5p | 290.6312604 | 253.1069751 | 1.148254647 |
| mmu-mir-1948, mmu-miR-1948-5p | 283.7495909 | 365.8921266 | 0.775500674 |
| mmu-mir-24-2, mmu-miR-24-2-5p | 275.4682212 | 345.5558383 | 0.797174264 |
| mmu-mir-20a, mmu-miR-20a-5p | 272.0381972 | 257.9374679 | 1.05466724 |
| mmu-mir-127, mmu-miR-127-3p | 267.8356669 | 353.9143642 | 0.756781001 |
| mmu-mir-365-1, mmu-miR-365-3p | 259.3805215 | 156.1557083 | 1.661037719 |
| mmu-mir-30d, mmu-miR-30d-3p | 258.7820452 | 260.4322451 | 0.993663612 |
| mmu-mir-195a, mmu-miR-195a-5p | 254.2853705 | 364.6171857 | 0.697403689 |
| mmu-mir-342, mmu-miR-342-3p | 253.5560098 | 277.9762637 | 0.912149859 |
| mmu-mir-150, mmu-miR-150-5p | 249.6881864 | 429.438016 | 0.581430095 |
| mmu-mir-145a, mmu-miR-145a-5p | 247.0929862 | 278.2538201 | 0.888012916 |
| mmu-mir-146a, mmu-miR-146a-5p | 246.4249991 | 333.8881993 | 0.738046447 |
| mmu-mir-484, mmu-miR-484 | 243.3616824 | 184.1814325 | 1.321314961 |
| mmu-mir-802, mmu-miR-802-3p | 242.6579881 | 88.10097651 | 2.754316668 |
| mmu-mir-30c-2, mmu-miR-30c-2-3p | 236.5490168 | 293.594098 | 0.805700858 |
| mmu-mir-17, mmu-miR-17-5p | 234.3023811 | 146.4882123 | 1.599462356 |
| mmu-mir-182, mmu-miR-182-5p | 228.9455275 | 331.6040339 | 0.690418403 |
| mmu-mir-142a, mmu-miR-142a-3p | 225.3419101 | 274.9008011 | 0.81972082 |
| mmu-mir-872, mmu-miR-872-5p | 217.31051 | 297.4167467 | 0.730659966 |
| mmu-let-7a-1, mmu-let-7a-1-3p | 212.6502909 | 259.8205411 | 0.818450651 |
| mmu-let-7c-2, mmu-let-7c-2-3p | 212.6502909 | 259.8205411 | 0.818450651 |
| mmu-mir-133a-1, mmu-miR-133a-3p | 207.9770553 | 143.471225 | 1.449608137 |
| mmu-mir-378a, mmu-miR-378a-5p | 204.3536872 | 157.3706126 | 1.298550497 |
| mmu-mir-144, mmu-miR-144-3p | 203.1147966 | 254.1173334 | 0.799295325 |
| mmu-mir-652, mmu-miR-652-3p | 198.1084679 | 185.9215303 | 1.065548824 |
| mmu-mir-141, mmu-miR-141-3p | 189.6815406 | 111.6023477 | 1.699619627 |
| mmu-mir-200a, mmu-miR-200a-3p | 184.9898596 | 214.1813296 | 0.863706748 |
| mmu-mir-744, mmu-miR-744-5p | 179.5654773 | 143.9408919 | 1.247494543 |
| mmu-mir-872, mmu-miR-872-3p | 162.6274076 | 163.8441067 | 0.992574044 |
| mmu-mir-22, mmu-miR-22-5p | 159.1328612 | 272.8545908 | 0.583214894 |
| mmu-mir-411, mmu-miR-411-5p | 158.9576813 | 255.3350902 | 0.622545382 |
| mmu-mir-338, mmu-miR-338-3p | 158.4027609 | 176.2440339 | 0.898769492 |
| mmu-mir-378b, mmu-miR-378b | 156.0852464 | 143.9478716 | 1.084317848 |
| mmu-mir-320, mmu-miR-320-3p | 155.3105661 | 84.32179909 | 1.841879179 |
| mmu-mir-328, mmu-miR-328-3p | 151.0763071 | 121.6179408 | 1.242220565 |
| mmu-mir-30e, mmu-miR-30e-3p | 143.9269525 | 136.7651898 | 1.052365392 |
| mmu-mir-28a, mmu-miR-28a-3p | 140.887681 | 153.1212918 | 0.920105097 |
| mmu-mir-455, mmu-miR-455-5p | 138.91057 | 132.6023522 | 1.047572443 |
| mmu-let-7f-2, mmu-let-7f-2-3p | 137.2326278 | 174.7560288 | 0.785281222 |
| mmu-mir-221, mmu-miR-221-3p | 133.4532069 | 99.16596352 | 1.345756167 |
| mmu-mir-450a-1, mmu-miR-450a-5p | 131.1431044 | 144.855184 | 0.905339393 |
| mmu-mir-139, mmu-miR-139-5p | 125.4123814 | 146.7557956 | 0.854565102 |
| mmu-mir-106b, mmu-miR-106b-5p | 120.4915732 | 105.9880684 | 1.13684092 |
| mmu-mir-322, mmu-miR-322-3p | 119.410744 | 122.0990774 | 0.977982361 |
| mmu-mir-1247, mmu-miR-1247-5p | 119.2721038 | 97.92157464 | 1.218037028 |
| mmu-mir-185, mmu-miR-185-5p | 110.4074302 | 63.47780109 | 1.739307732 |
| mmu-mir-192, mmu-miR-192-3p | 109.0277086 | 112.4280916 | 0.969755041 |
| mmu-mir-455, mmu-miR-455-3p | 108.3962564 | 92.14777174 | 1.176330738 |
| mmu-mir-361, mmu-miR-361-3p | 108.1124374 | 89.94055123 | 1.202043304 |
| mmu-mir-152, mmu-miR-152-5p | 105.8941898 | 77.89880046 | 1.35938152 |
| mmu-mir-181b-1, mmu-miR-181b-5p | 101.4557126 | 125.4616349 | 0.808659258 |
| mmu-mir-92a-1, mmu-miR-92a-1-5p | 94.44869089 | 144.8896924 | 0.651866184 |
| mmu-mir-339, mmu-miR-339-5p | 90.26563976 | 79.5628127 | 1.134520471 |
| mmu-let-7b, mmu-let-7b-3p | 90.00369215 | 141.5001101 | 0.636068001 |
| mmu-mir-676, mmu-miR-676-3p | 85.43489138 | 98.14311526 | 0.870513343 |
| mmu-mir-210, mmu-miR-210-3p | 84.54540256 | 86.44247454 | 0.978053937 |
| mmu-mir-32, mmu-miR-32-5p | 81.73547658 | 70.77759733 | 1.154821295 |
| mmu-mir-194-2, mmu-miR-194-2-3p | 80.38004029 | 87.79038123 | 0.915590514 |
| mmu-mir-326, mmu-miR-326-3p | 78.1934948 | 81.92456024 | 0.954457303 |
| mmu-mir-222, mmu-miR-222-3p | 78.16179273 | 48.90224742 | 1.598327211 |
| mmu-mir-23a, mmu-miR-23a-3p | 78.05814672 | 97.3408792 | 0.801905092 |
| mmu-mir-128-1, mmu-miR-128-3p | 75.40215285 | 63.82891876 | 1.181316468 |
| mmu-mir-149, mmu-miR-149-5p | 74.26625804 | 53.38414667 | 1.391166904 |
| mmu-mir-101a, mmu-miR-101a-5p | 73.4622365 | 35.55709867 | 2.066035735 |
| mmu-mir-128-2, mmu-miR-128-3p | 72.29026054 | 63.07868485 | 1.146033097 |
| mmu-mir-671, mmu-miR-671-3p | 70.74568437 | 72.70368257 | 0.973068789 |
| mmu-mir-5099, mmu-miR-5099 | 67.37773754 | 93.04349801 | 0.7241531 |
| mmu-mir-19a, mmu-miR-19a-3p | 66.34219658 | 70.29592964 | 0.943755875 |
| mmu-mir-214, mmu-miR-214-3p | 65.41663142 | 85.28508995 | 0.767034794 |
| mmu-mir-434, mmu-miR-434-3p | 64.1213119 | 85.90578405 | 0.746414373 |
| mmu-mir-1198, mmu-miR-1198-5p | 64.02008947 | 61.18259065 | 1.046377553 |
| mmu-mir-136, mmu-miR-136-3p | 59.40056591 | 77.00500554 | 0.771385775 |
| mmu-mir-148b, mmu-miR-148b-5p | 59.33191412 | 42.80585844 | 1.386069951 |
| mmu-mir-339, mmu-miR-339-3p | 58.82529768 | 50.49573625 | 1.164955738 |
| mmu-mir-1981, mmu-miR-1981-3p | 58.20126216 | 25.12083022 | 2.316852654 |
| mmu-mir-146b, mmu-miR-146b-5p | 57.05485025 | 63.34478871 | 0.900703142 |
| mmu-mir-345, mmu-miR-345-5p | 56.26441482 | 47.05117566 | 1.195813155 |
| mmu-mir-511, mmu-miR-511-3p | 56.05297166 | 65.54794381 | 0.855144622 |
| mmu-let-7f-1, mmu-let-7f-1-3p | 52.79021624 | 62.01184163 | 0.851292509 |
| mmu-mir-106b, mmu-miR-106b-3p | 52.5011544 | 42.81536231 | 1.226222355 |
| mmu-mir-33, mmu-miR-33-5p | 51.61188392 | 33.80054461 | 1.526954211 |
| mmu-mir-421, mmu-miR-421-3p | 48.255973 | 42.29969999 | 1.140811235 |
| mmu-mir-136, mmu-miR-136-5p | 45.26739286 | 45.8943148 | 0.986339878 |
| mmu-mir-26b, mmu-miR-26b-3p | 45.03649709 | 66.96539096 | 0.672533923 |
| mmu-mir-340, mmu-miR-340-3p | 44.85415026 | 35.08845196 | 1.27831659 |
| mmu-mir-125b-2, mmu-miR-125b-2-3p | 42.74544181 | 44.12176366 | 0.968806282 |
| mmu-mir-187, mmu-miR-187-3p | 42.65644224 | 45.67474737 | 0.93391742 |
| mmu-mir-664, mmu-miR-664-3p | 42.34792778 | 37.85481601 | 1.118693267 |
| mmu-mir-221, mmu-miR-221-5p | 42.01075368 | 30.13089903 | 1.394274815 |
| mmu-mir-99a, mmu-miR-99a-3p | 41.57213399 | 66.55826755 | 0.624597597 |
| mmu-mir-132, mmu-miR-132-3p | 41.47156179 | 44.28281845 | 0.93651586 |
| mmu-mir-361, mmu-miR-361-5p | 39.9009583 | 31.27825602 | 1.275677208 |
| mmu-mir-362, mmu-miR-362-3p | 39.5937491 | 38.97567181 | 1.015858028 |
| mmu-mir-374b, mmu-miR-374b-5p | 38.11651945 | 38.28294033 | 0.995652871 |
| mmu-mir-98, mmu-miR-98-3p | 35.11767194 | 30.07838314 | 1.167538553 |
| mmu-mir-96, mmu-miR-96-5p | 35.01774997 | 40.10151415 | 0.873227625 |
| mmu-mir-34a, mmu-miR-34a-5p | 34.98713003 | 43.68413547 | 0.800911582 |
| mmu-mir-429, mmu-miR-429-3p | 34.24611212 | 36.17128228 | 0.946776281 |
| mmu-mir-874, mmu-miR-874-3p | 33.96601717 | 29.76828433 | 1.141013597 |
| mmu-mir-1249, mmu-miR-1249-3p | 32.38688347 | 44.23480262 | 0.732158426 |
| mmu-mir-676, mmu-miR-676-5p | 31.95068256 | 25.50844942 | 1.25255291 |
| mmu-mir-212, mmu-miR-212-5p | 30.87140363 | 37.38567283 | 0.825754929 |
| mmu-mir-381, mmu-miR-381-3p | 29.51246163 | 42.19064734 | 0.699502461 |
| mmu-mir-7a-1, mmu-miR-7a-5p | 28.70781645 | 18.5417906 | 1.548276381 |
| mmu-mir-7a-2, mmu-miR-7a-5p | 28.70781645 | 18.5417906 | 1.548276381 |
| mmu-mir-223, mmu-miR-223-3p | 28.45986026 | 30.24292779 | 0.941041835 |
| mmu-mir-664, mmu-miR-664-5p | 28.21146737 | 33.87708205 | 0.832759661 |
| mmu-mir-744, mmu-miR-744-3p | 28.03982461 | 23.17472703 | 1.209931171 |

To examine if hepatic Ago2 regulates biogenesis of these MD-miRNAs, Applicant measured the expression levels of each mature and primary miRNA (pri-miRNA) employing TaqMan probe-based gene expression analysis. Mature miRNA levels of miR-802, miR-107/miR-103, miR-130a, and miR-148a/148b/152, are reduced in L-Ago2 KO liver (FIG. 1F), despite intact expression levels of their pri-miRNAs (FIG. 1G). These results suggest that the miRNA maturation process is impaired in the Ago2-deficient liver. To further confirm this regulation, Applicant utilized Ago2-deficient mouse embryonic fibroblasts (MEFs) reconstituted with wild type Ago2 -defective mutant Ago2 (Ago2 D669A, or “DA,” containing an aspartate to alanine substitution at residue 669)²²⁻²⁴. Expression levels of these MD-miRNAs were increased by reconstitution of Ago2 WT (FIG. 9B). Importantly, Ago2 D669A mutant only partially induced expression of key MD-miRNAs including miR-107, miR-103, and miR-130a, while expression of miR-148b required Ago2 but not its slicer activity (FIG. 9B), suggesting a possible involvement of Ago2 slicer activity in metabolic regulation. While the loss of Dicer also causes a reduction of MD-miRNAs (FIG. 9C), these results support a crucial role of Ago2 in the biogenesis of a set of MD-miRNAs.

While Dicer recognizes the 5′ phosphate end and 2-nucleotide 3′ overhang structure of precursor miRNA for precise and effective biogenesis of miRNAs^{30,31}, recent studies have provided different mechanistic insights into the Ago2-mediated processing of miRNA. One of the proposed characteristics of miRNAs processed by Ago2 is that their precursors have a relatively shorter loop size that likely prevents recognition by Dicer. Moreover, these precursors have no mis-matching at position 10 or 11 between guide and passenger strands32. The miRNAs with reduced expression in L-Ago2 KO liver tended to have shorter loop sizes than those induced by L-Ago2 KO liver, and had no mis-matching at positions 10 or 11 (FIG. 9D-F). Additionally, while the total number of reads for MD-miRNAs were drastically reduced in L-Ago2 KO liver, modifications to these miRNAs, including uridylation and adenylation, occurred with similar frequency between the genotypes (as follows). This information suggests that there may be structural similarities among MD-miRNAs that require Ago2 for maturation.

| | |
|---|---|
| WT | KO |

miR-802-5p (all)

| | | |
|---|---|---|
| 3 | 1 | AUCAGUAACAAAGAUUCAUCCUUG |
| 1 | 0 | AUCAGUAACAAGAUUCAUCCUUGU |
| 1 | 0 | GUUCAGUAACAAAGAUUCAUCCUUG |
| 9 | 1 | AUCAGUAACAAAGAUUCAUCCUU |
| 1 | 0 | GACGAUCUCAGUAACAAAGAUUCAUDDUU |
| 1 | 0 | UCAGUAACAAAGAUUCAUCCUUGUU |
| 5 | 0 | UCAGUAACAAAGAUUCAUCCUUGG |
| 1 | 0 | UCAGUAACAAAGAUUCAUCCUUAU |
| 46 | 9 | UCAGUAACAAAGAUUCAUCCUUGA |
| 888 | 124 | UCAGUAACAAAGAUUCAUCCUU |
| 2 | 1 | UCAGUAACAAAGAUUCAUCCUUA |
| 43 | 5 | UCAGUAACAAAGAUUCAUCCUUU |
| 1 | 0 | UCAGUAACAAAGAUUCAUCCUUGGAU |
| 2 | 0 | UCAGUAACAAAGAUUCAUCCUUGAC |
| 1 | 0 | CCCGUGGUCAGUAACAAAGAUGCAUCCUU |
| 1 | 0 | UCAGUAACAAAGAUUCAUCCUUGC |
| 3 | 1 | CUCAGUAACAAAGAUUCAUCCUUG |
| 1 | 0 | UCAGUAACAAAGAUUCAUCCUUAGU |
| 1 | 0 | UCAGUAACAAAGAUUGCAUCCUUGGAAUUCUUGGGUUGCAAGGAACUCCAG |
| 12 | 2 | UCAGUAACAAAGAUUCAUCCUUGUGU |
| 273 | 71 | UCAGUAACAAAGAUUCAUCCUUGU |
| 5584 | 1364 | UCAGUAACAAAGAUUCAUCCUUG |
| | | . .UUUGCAAUCAGUAACAAAGAUUCAUCCUUGUGUCAAUCAUACAACACGAGAGUCUUU. . |

Genome Matched

| | | |
|---|---|---|
| 888 | 124 | UCAGUAACAAAGAUUCAUCCUU |
| 5584 | 1364 | 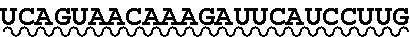 |
| 273 | 71 | UCAGUAACAAAGAUUCAUCCUUGU |
| 12 | 2 | UCAGUAACAAAGAUUCAUCCUUGUGU |
| 9 | 1 | AUCAGUAACAAAGAUUCAUCCUU |
| 3 | 1 | AUCAGUAACAAAGAUUCAUCCUUG |
| 1 | 0 | AUCAGUAACAAAGAUUCAUCCUUGU |
| | | 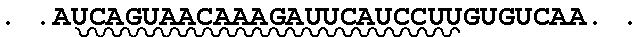 |

U Addition

| | | |
|---|---|---|
| 888 | 124 | 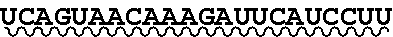 |
| 43 | 5 | UCAGUAACAAAGAUUAUCCUU |
| 5584 | 1364 | 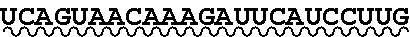 |
| 273 | 71 | UCAGUAACAAAGAUUCAUCCUUGU |
| 1 | 0 | UCAGUAACAAAGAUUCAUCCUUGUU |
| 12 | 2 | UCAGUAACAAAGAUUCAUCCUUGUGU |
| 9 | 1 | AUCAGUAACAAAGAUUCAUCCUU |
| 3 | 1 | AUCAGUAACAAAGAUUCAUCCUUG |
| 1 | 0 | AUCAGUAACAAAGAUUCAUCCUUGU |
| | | 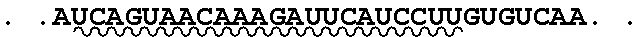 |

A Addition

| | | |
|---|---|---|
| 888 | 124 | UCAGUAACAAAGAUUCAUCCUU |
| 2 | 1 | UCAGUAACAAGAUUCAUCCUUA |
| 5584 | 1364 | 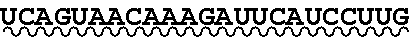 |
| 46 | 9 | UCAGUAACAAGAUUCAUCCUUGA |
| 273 | 71 | UCAGUAACAAAGAUUCAUCCUUGU |
| 12 | 2 | UCAGUAACAAAGAUUCAUCCUUGUGU |
| 9 | 1 | AUCAGUAACAAAGAUUCAUCCUU |
| 3 | 1 | AUCAGUAACAAAGAUUCAUCCUUG |
| 1 | 0 | AUCAGUAACAAAGAUUCAUCCUUGU |
| | | 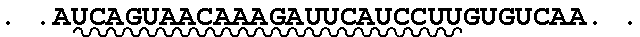 |

miR-107-3p (all)

| | | |
|---|---|---|
| 3 | 1 | AGCAGCAUUGUACAGGGCUAUCACU |
| 241 | 76 | AGCAGCAUUGUACAGGGCUAUCA |
| 1 | 0 | AGCAGCAUUGUACAGGGCUAUCAAUG |
| 2 | 0 | AGCAGCAUUGUACAGGGCUAUCAGU |
| 6 | 4 | AGCAGCAUUGUACAGGGUAUCAUU |
| 1 | 0 | AGCAGCAUUGUACAGGGUAUCAACA |
| 3 | 1 | AGCAGCAUUGUACAGGGUAUCAA |
| 1 | 0 | CAGCAGCAUUGUACAGGGCUAUCAA |
| 116 | 46 | AGCAGCAUUGUACAGGGUAUCAA |
| 2 | 0 | AGCAGCAUUGUACAGGGCUAUCAGA |
| 1 | 0 | AGCAGCAUUGUACAGGGCUAUCAAUCU |
| 0 | 1 | AGCAGCAUUGUACAGGGCUAUCAAGAUUUU |
| 36 | 7 | AGCAGCAUUGUACAGGGCUAUCAU |
| 10 | 1 | AGCAGCAUGUACAGGGCUAUCAAU |
| 1 | 0 | AGCAGCAUUGUACAGGGCUAUCAAG |
| 2 | 0 | AGCAGCAUUGUACAGGGCUAUCAAGU |
| 3 | 4 | AGCAGCAUUGUACAGGGCUAUCAG |
| | | . .AAGCAGCAUUGUACAGGGCAUAUCAAAGCACAGAGAGC |

Genome Matched

| | | |
|---|---|---|
| 241 | 76 | 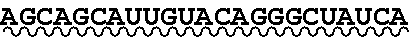 |
| 3 | 1 | AGCAGCAUUGUACAGGGCUAUCAAA |
| 1 | 0 | CAGCAGCAUUGUACAGGGUAUCAA |
| 116 | 46 | AGCAGCAUUGUACAGGGCUAUCAA |
| | | 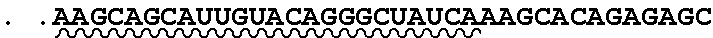 |

U Addition

| | | |
|---|---|---|
| 241 | 76 | 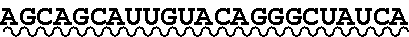 |
| 36 | 7 | AGCAGCAUUGUACAGGGCUAUCAU |
| 6 | 4 | AGCAGCAUUGUACAGGGUAUCAUU |
| 116 | 46 | AGCAGCAUUGUACAGGGUAUCAA |
| 10 | 1 | AGCAGCAUUGUACAGGGCUAUCAAU |
| 3 | 1 | AGCAGCAUUGUACAGGGCUAUCAA |
| | | 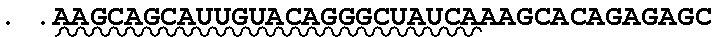 |

A Addition

| | | |
|---|---|---|
| 241 | 76 | 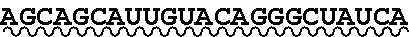 |
| 3 | 1 | AGCAGCAUUGUACAGGGCUAUCAAA |
| 1 | 0 | CAGCAGCAUUGUACAGGGCUAUCAA |
| 116 | 46 | AGCAGCAUUGUACAGGGCUAUCAA |
| | | 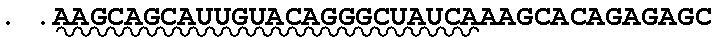 |

miR-103-3p
All

| | | |
|---|---|---|
| 1 | 1 | AGCAGCAUUGUACAGGGCUAUGAUAU |
| 2 | 2 | AGCAGCAUUGUACAGGGUAUGAAU |
| 402 | 226 | AGCAGCAUUGUACAGGGCUAUGAU |
| 1 | 0 | AGCAGCAUUGUACAGGGCUAUGACA |
| 34 | 15 | AGCAGCAUUGUACAGGGCUAUGAUU |
| 3224 | 1949 | 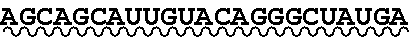 |
| 0 | 1 | AGCAGCAUUGUACAGGGUAUGAUUUUU |
| 3 | 0 | AGCAGCAUUGUACAGGGCUAUGAUCU |
| 1 | 1 | AGCAGCAUUGUACAGGGCUAUGAAGA |
| 1 | 0 | CAGCAGCAUUGUACAGGGCUAUGAUU |
| 10 | 1 | AGCAGCAUUGUACAGGCUAUGAU |
| 4 | 4 | AGCAGCAUUGUACAGGGCUAUGAAUU |
| 0 | 1 | AGCAGCAUUGUACAGGGCUAUGAAAGA |
| 0 | 1 | AGCAGCAUUGUACAGGGCUAUGAUUCU |
| 2 | 0 | AGCAGCAUUGUACAGGGUAUGAGAAU |
| 1 | 0 | AGCAGCAUUGUACAGGGCUAUGGAAUUCUCGGGCCA |
| 1 | 5 | AGCAGCAUUGUACAGGGCUAUGAUG |
| 0 | 1 | AGCAGCAUUGUACAGGCUAUGAGUU |
| 1 | 0 | AGCAGCAUUGUACAGGGCUAUGAGCUC |
| 3 | 0 | AGCAGCAUUGUACAGGGCUAUGAC |
| 10 | 5 | AGCAGCAUUGUACAGGGCUAUGAGA |
| 8 | 6 | AGCAGCAUUGUACAGGGCUAUGAGU |
| 2 | 0 | AGCAGAUUGUACAGGGCUAUGAUUU |
| 12 | 5 | AGCAGCAUUGUACAGGGCUAUGAG |
| 1 | 0 | AGCAGCAUUGUACAGGGCUAUGAAGC |
| 6 | 4 | AGCAGCAUUGUACAGGGCUAUGACU |
| 39 | 14 | AGCAGCAUUGUACAGGGCUAUGAAA |
| 1 | 0 | AGCAGCAUUGUACAGGGCUAUGAUAA |
| 124 | 75 | AGCAGCAUUGUACAGGGCUAUGAA |
| 1 | 0 | CAGCAGCAUUGUACAGGGCUAUGA |
| 20 | 12 | AGCAGCAUUGUACAGGGCUAUGAAU |
| 1 | 3 | AGCAGCAUUGUACAGGGCUAUGAGAA |
| 2 | 0 | AGCAGCAUUGUACAGGGCUAUGAUUUU |
| 1 | 2 | AGCAGCAUUGUACAGGGCUAUGAUC |
| 0 | 1 | AGCAGCAUUGUACAGGGCUAUGAUGUU |

| | |
|---|---|
| mir-103-1 | 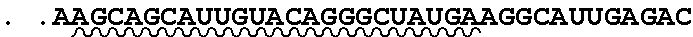 |
| mir-103-2 | 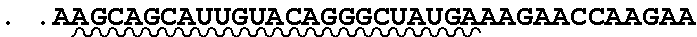 |

Genome Matched

| | | |
|---|---|---|
| 3224 | 1949 | 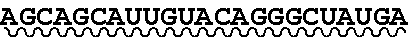 |
| 124 | 75 | AGCAGCAUUGUACAGGGCUAUGAA |
| 39 | 14 | AGCAGCAUUGUACAGGGCUAUGAA |
| 0 | 1 | AGCAGCAUUGUACAGGGCUAUGAAAGA |
| | | 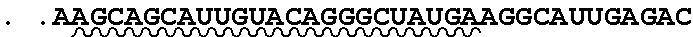 |
| | | 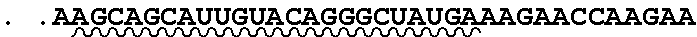 |

U Addition

| | | |
|---|---|---|
| 3224 | 1949 | 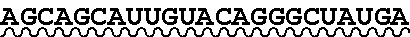 |
| 402 | 226 | AGCAGCAUUGUACAGGGCUAUGAU |
| 34 | 15 | AGCAGCAUUGUACAGGGCUAUGAUU |
| 2 | 0 | AGCAGCAUUGUACAGGGUAUGAUUU |
| 2 | 0 | AGCAGCAUUGUACAGGGUAUGAUUU |
| 0 | 1 | AGCAGCAUUGUACAGGGCUAUGAUUUUU |
| 124 | 75 | AGCAGCAUUGUACAGGGCUAUGAA |
| 20 | 12 | AGCAGCAUUGUACAGGGCUAUGAAU |
| 4 | 4 | AGCAGCAUUGUACAGGGCUAUGAAUU |
| 39 | 14 | AGCAGCAUUGUACAGGGUAUGAAA |
| 2 | 2 | AGCAGCAUUGUACAGGGCUAUGAAAU |
| 0 | 1 | AGCAGCAUUGUACAGGGCUAUGAAAGA |
| | | 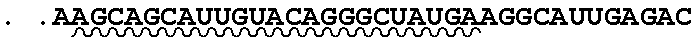 |
| | | 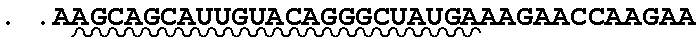 |

A Addition

| | | |
|---|---|---|
| 3224 | 1949 | AGCAGCAUUGUACAGGGCUAUGA |
| 124 | 75 | AGCAGCAUUGUACAGGGCUAUGAA |
| 39 | 14 | AGCAGCAUUGUACAGGGCUAUGAA |
| 1 | 1 | AGCAGCAUUGUACAGGGCUAUGAAGA |
| 0 | 1 | AGCAGCAUUGUACAGGGCUAUGAAAGA |
| | | 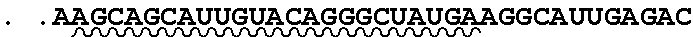 |
| | | 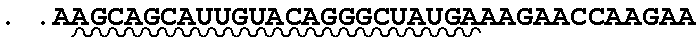 |

Inactivation of Hepatic Ago2 Improves Systemic Glucose Metabolism

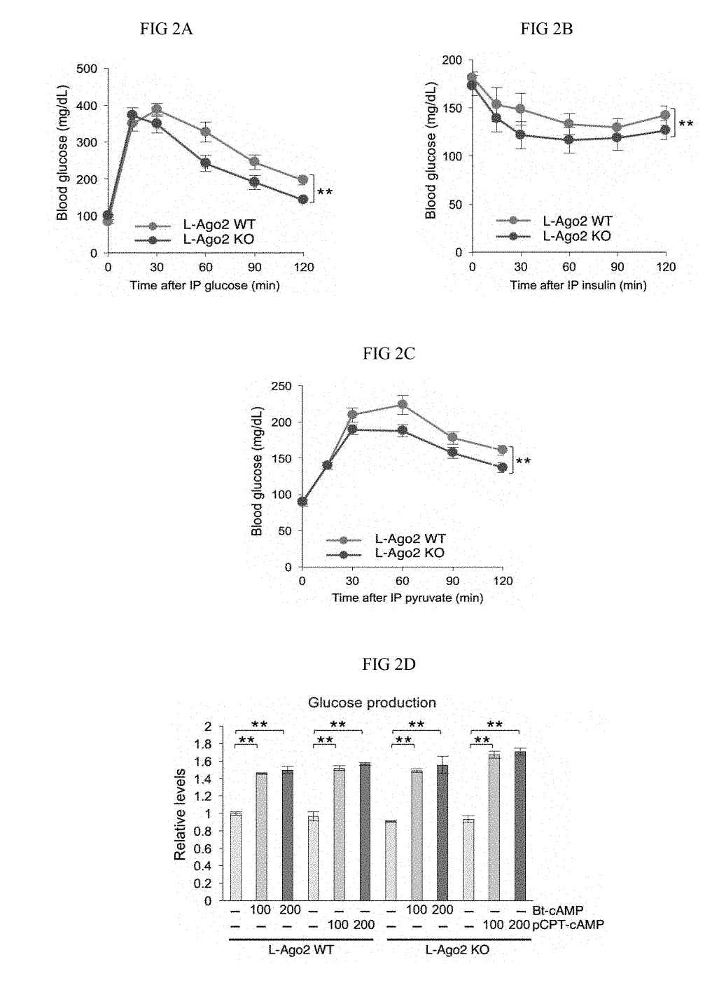

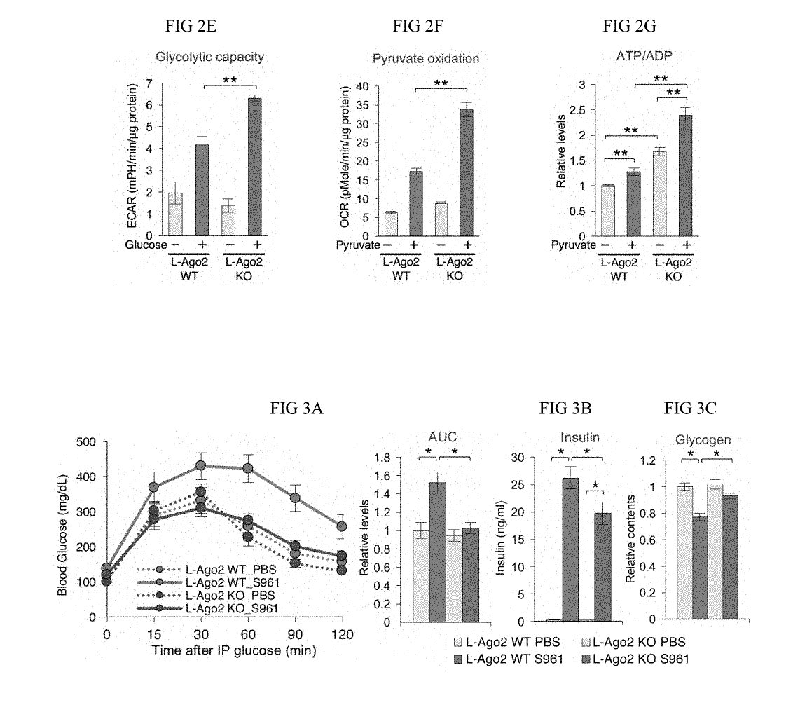

Considering the miRNA enrichment pathway analysis indicated that hepatic Ago2 is implicated in glucose metabolism, Applicant then investigated Ago2's role in regulating this regard. Applicant observed that L-Ago2 KO mice fed normal chow diet (NCD) exhibited enhanced capacities for glucose metabolism, as assessed by glucose, insulin, and pyruvate tolerance tests (GTT, ITT, and PTT) after 20 weeks of age (FIG. 9A-C). These results suggest that hepatic Ago2 deficiency improves insulin sensitivity and inhibits gluconeogenesis, leading to glucose tolerance. To investigate how Ago2 regulates glucose metabolism, Applicant first examined capacities of hepatic gluconeogenesis in Ago2-deficient primary hepatocytes. Glucose production stimulated by dibutyryl cyclic AMP (Bt-cAMP) or 8-(4-Chlorophenylthio)adenosine 3′,5′-cyclic monophosphate (pCPT-cAMP), hydrolysis-resistant cAMP analogs, was similarly induced between genotypes (FIG. 2D), suggesting that Ago2-deficiency affects insulin-mediated suppression of gluconeogenesis rather than gluconeogenic capacities. Applicant additionally asked if Ago2 regulates the fundamental catabolic capacities of hepatocytes. To examine the glycolytic rate, the extracellular acidification rate (ECAR) was determined in primary hepatocytes upon addition of glucose. In the presence of oligomycin, which blocks mitochondrial ATP production and promotes maximal rates of glycolysis, Ago2-deficient hepatocytes showed a higher increase in ECAR compared to controls (FIG. 9E). Glycolysis produces pyruvate that is used as an energy source for the citric acid cycle.

To determine whether Ago2 regulates oxidation of pyruvate, Applicant measured mitochondrial oxygen consumption rate (OCR) in WT and Ago2-deficient hepatocytes in the presence of pyruvate. Upon the addition of the protonophore carbonyl cyanide 4-(trifluoromethoxy) phenylhydrazone (FCCP), which uncouples oxidative phosphorylation from electron transport to allow maximal respiration, Ago2-deficient hepatocytes greatly upregulated oxygen consumption compared to WT controls (FIG. 2F). In a complementary approach, Applicant also measured ATP content/ADP content in these hepatocytes in the absence or presence of pyruvate. The basal ATP/ADP ratio in Ago2-deficient hepatocytes was higher than that in WT controls, and this difference was even greater upon addition of pyruvate (FIG. 2G). Taken together, these data indicate that hepatic Ago2 functions to enhance gluconeogenesis and suppress glucose oxidation while its inactivation results in increased glucose-driven energy production.

Hepatic Ago2 Regulates Glucose Metabolism in Insulin Insufficiency

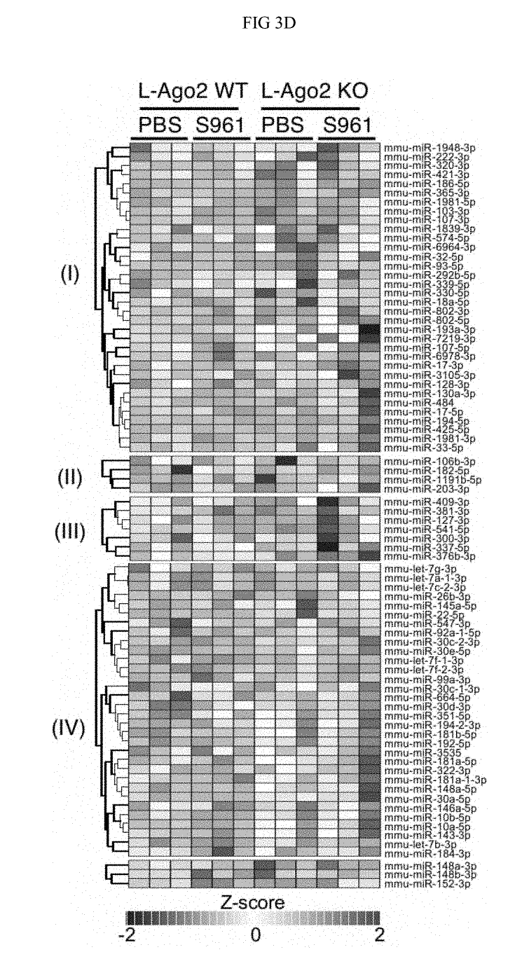

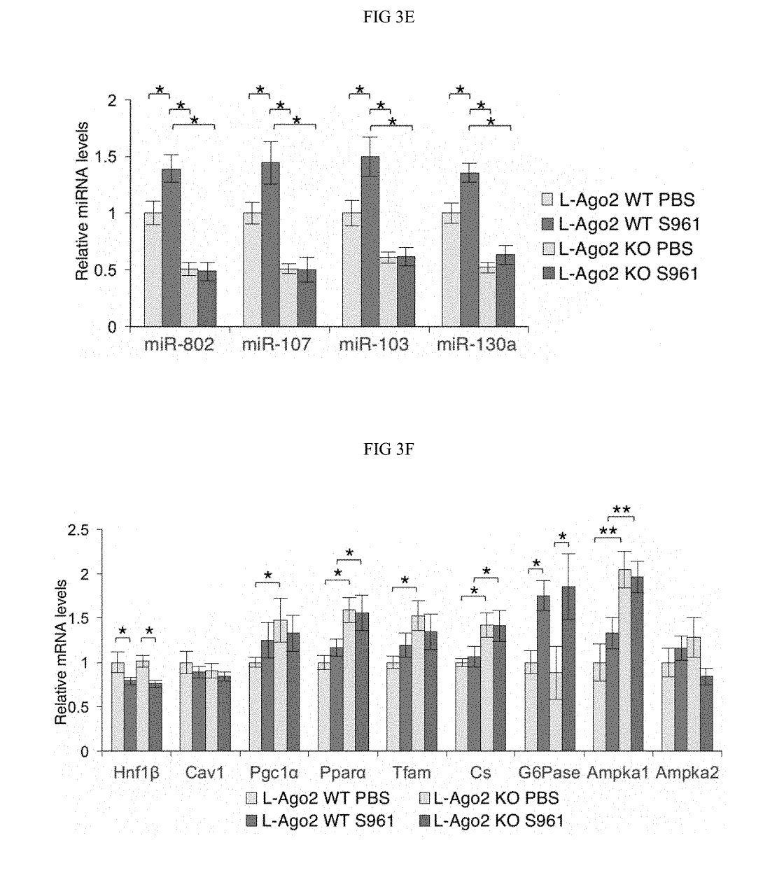

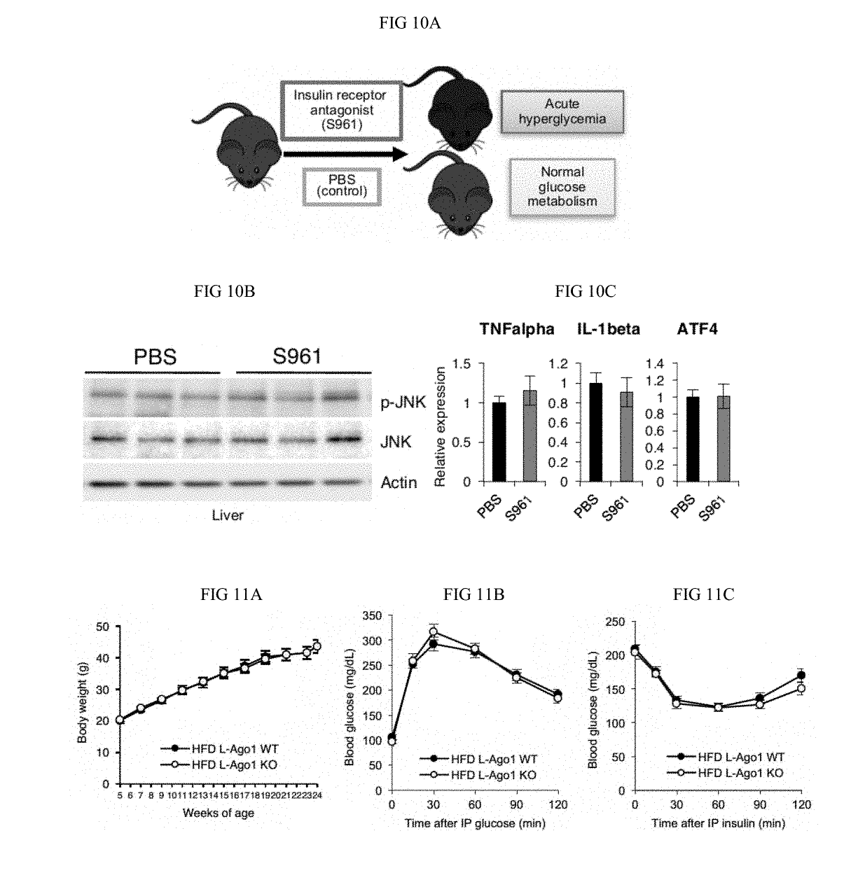

If hepatic Ago2-deficiency improves systemic glucose metabolism by suppressing gluconeogenesis and accelerating glucose oxidation in the liver, other diabetic conditions may also be improved by hepatic Ago2-deficiency. To examine this possibility, Applicant employed a pharmacological model by administering the insulin antagonist peptide, S961 (43 amino acids in length) (FIG. 10A). S961 binds to the insulin receptor and blocks insulin signaling in vivo to acutely induce hyperglycemia^{33,34}. This model can partially mimic a condition of type 1 diabetes in metabolic cells such as hepatocytes, which therefore enhances hepatic glucose production and suppresses glucose oxidation despite holding an intact insulin signaling cascade. This model allowed us to further assess the role of hepatic Ago2 in glycemic control without the potential confounding effects of body weight and adiposity. In addition, Applicant confirmed that S961 treatment did not induce typical inflammatory responses, such as elevated JNK activation and inflammatory cytokines, which potentially disrupt glucose metabolism (FIGS. 10B and 10C). After infusing S961 into L-Ago2 WT and L-Ago2 KO mice fed standard chow diet at 12 weeks of age, Applicant examined the effect of hepatic Ago2-deficiency on S961-induced deterioration of glucose metabolism. At this age, while glucose metabolism assessed by GTT was comparable between the genotypes in a PBS-treated control group, S961 treatment caused hyperglycemia in WT mice one week after treatment (FIG. 3A). Remarkably, L-Ago2 KO mice are resistant to S961-induced glucose intolerance after one week of treatment (FIG. 3A). S961 treatment caused hyperinsulinemia and glycogenolysis in WT mice after two weeks of treatment (FIGS. 3B and 3C). However, S961 treatment of L-Ago2 KO mice resulted in a lower induction of plasma insulin levels and higher hepatic glycogen contents compared with control mice (FIGS. 3B and 3C). These data indicate that inactivation of hepatic Ago2 improves systemic glucose metabolism in the condition of insulin insufficiency.

Expression profiles that assessed the effect of insulin insufficiency on miRNAs in the liver categorized four different classes of miRNAs; (I) dominantly expressed in L-Ago2 WT, (II) induced by S961 in both L-Ago2 WT and KO, (III) suppressed by S961 in both L-Ago2 WT and KO, and (IV) expressed in L-Ago2 KO (FIG. 3d). Importantly, S961 treatment did not modulate expression levels of miRNAs categorized in class (IV) in both L-Ago2 WT and KO liver. Conversely, several miRNAs in class (I) that contains MD-miRNAs including miR-802, miR-103/107, and miR-130a were strikingly induced by S961 treatment in the WT liver in an Ago2-dependent manner (FIGS. 3D and 3E). These analyses implicate hepatic Ago2-dependent miRNAs play a role in the disruption of glucose metabolism under the condition of insulin insufficiency. Despite the decrease of miR-802 and miR-103/107 expression in the Ago2-deficient condition, expression levels of their known targets, Hnf1β and Cav1, were comparable between the genotypes even in PBS-treated groups at this age (FIG. 3F). While expression of glucose-6-phosphatase (G6Pase), a gluconeogenic gene, was similarly induced between the genotypes (FIG. 3F), expression of genes critical for mitochondrial function, such as Pgc1α, peroxisome proliferator-activated receptor alpha Ppar, mitochondrial transcription factor (Tfam), and citrate synthase (Cs) were higher in the liver of L-Ago2 KO mice. These results, along with the Ago2-deficient hepatocyte analyses, suggest that Ago2 regulates the program of a set of miRNAs involved in energy metabolism, which may be associated with enhancement of gluconeogenesis and suppression of glycolysis and hepatic mitochondrial oxidation induced by insulin insufficiency.

Critical Roles of Hepatic Ago2 in Energy Metabolism on High Fat-Diet Challenge

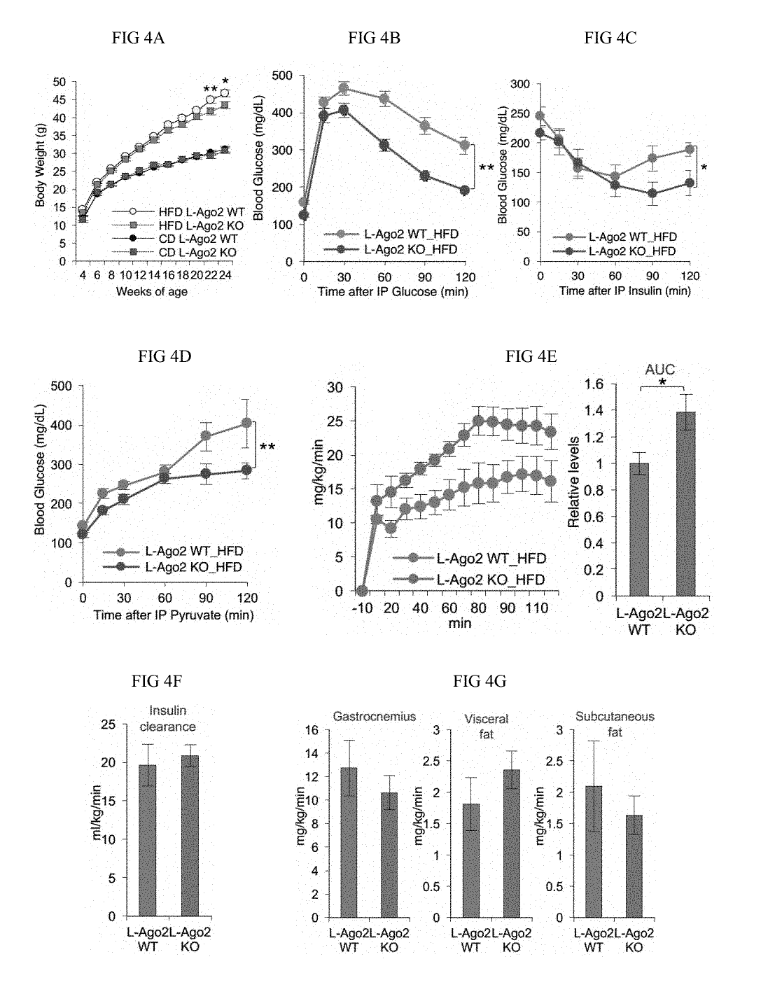

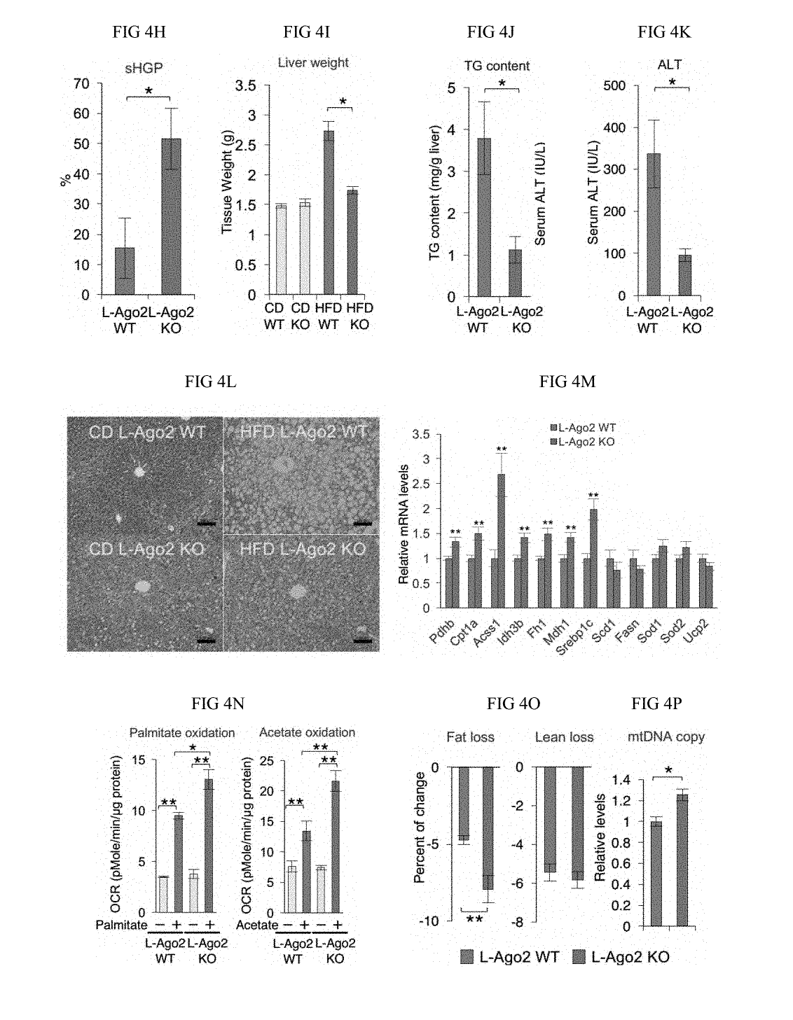

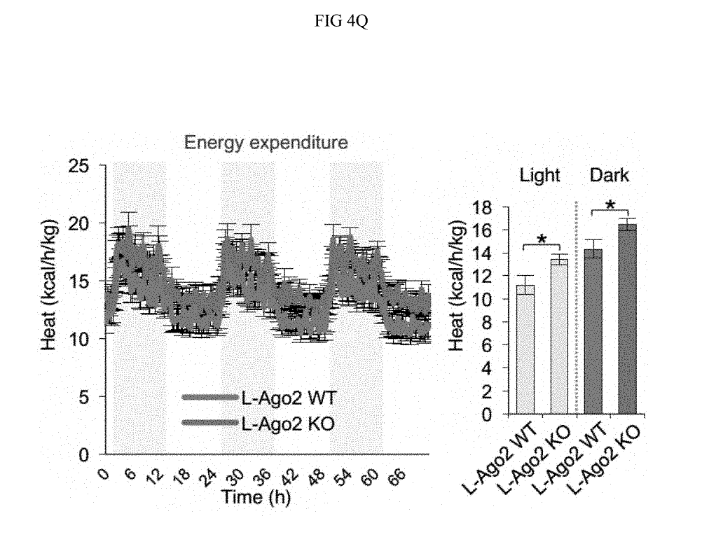

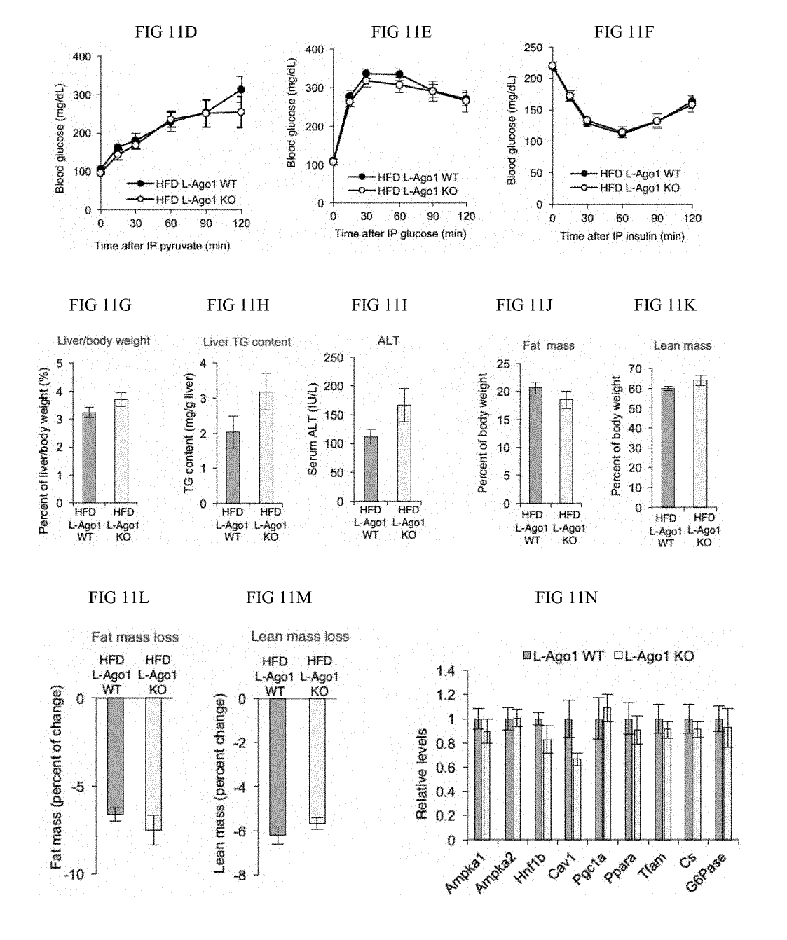

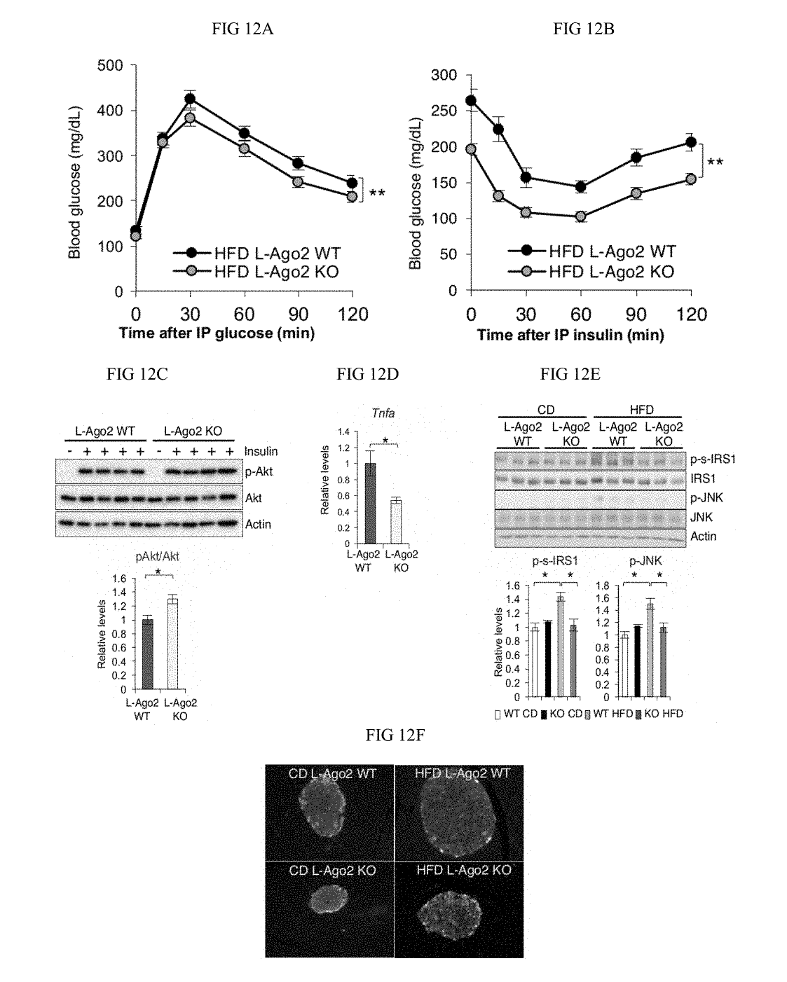

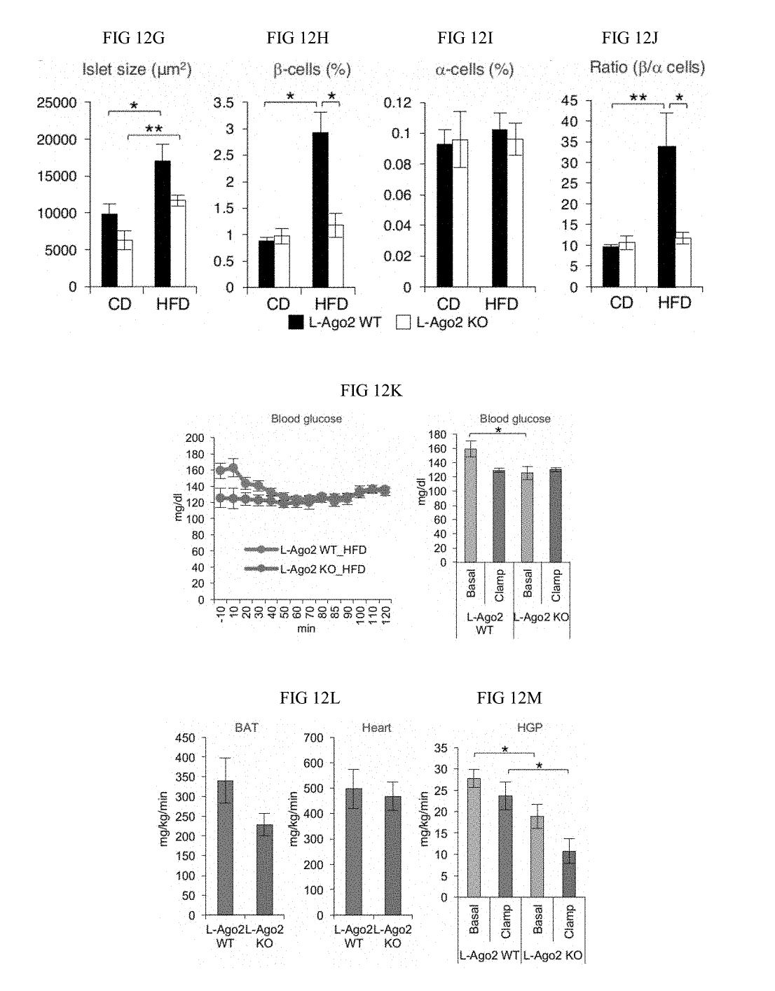

Given that Ago2's function is linked to glucose metabolism, Applicant asked if nutrient challenge might accentuate Ago2's role in metabolic regulation. Applicant thus employed a high-fat diet (HFD)-induced obesity model that induces insulin resistance, glucose intolerance, and hepatic steatosis. Applicant placed L-Ago2 KO, and L-Ago1 KO, and control WT mice on HFD or a control diet (CD), commencing at four weeks of age (FIG. 4A and FIG. 11A). Of note, the body weights of the HFD-fed L-Ago2 KO mice became lower than that of controls and the difference reached statistical significance at 22 weeks of age (FIG. 4A). Conversely, the body weight of L-Ago1 KO mice was comparable to controls in the HFD condition (FIG. 11A). The improvements in glucose metabolism observed in the HFD-fed L-Ago2 KO were even more pronounced when compared to the CD-feeding condition, as assessed by GTT, ITT, and PTT (FIG. 4B-4D, FIGS. 12A and 12B). These improvements were not observed in L-Ago1 KO fed HFD (FIG. 11B-11F). Consistent with improved systemic glucose tolerance and insulin sensitivity in L-Ago2 KO mice, there was a statistically significant increase in insulin-stimulated Protein kinase B (Akt) phosphorylation (FIG. 12C), accompanied by lowered levels of inflammatory markers, including expression of Tnfa, activation of c-Jun N-terminal kinase (JNK), and inhibitory phosphorylation levels of insulin receptor substrate 1 (IRS1) (FIGS. 12D and 12E), in the liver of L-Ago2 KO. In addition, HFD-induced pancreatic (3-cell proliferation and islet hypertrophy were attenuated compared to L-Ago2 WT mice (FIG. 12F-J), supporting that hepatic Ago2-deficiency improves insulin sensitivity in the pathogenesis of obesity.

To further demonstrate the role of hepatic Ago2, Applicant performed the hyperinsulinemic-euglycemic clamp study to examine the whole-body glucose metabolism and insulin sensitivity. Glucose infusion rates (GIR) during the clamp studies indicated that L-Ago2 KO mice required significantly higher levels of glucose infusion to maintain blood glucose consistent with increased insulin sensitivity (FIG. 4E and FIG. 12K). Insulin clearance and glucose uptakes in gastrocnemius muscle, visceral and subcutaneous fat, brown adipose tissue, and heart were comparable (FIGS. 4F and 4G and FIG. 12L). Conversely, hepatic glucose production was significantly suppressed in L-Ago2 KO (FIG. 4H and FIG. 12M). These studies indicate that the liver is the main locus responsible for improving systemic insulin sensitivity and glucose metabolism in L-Ago2 KO mice.

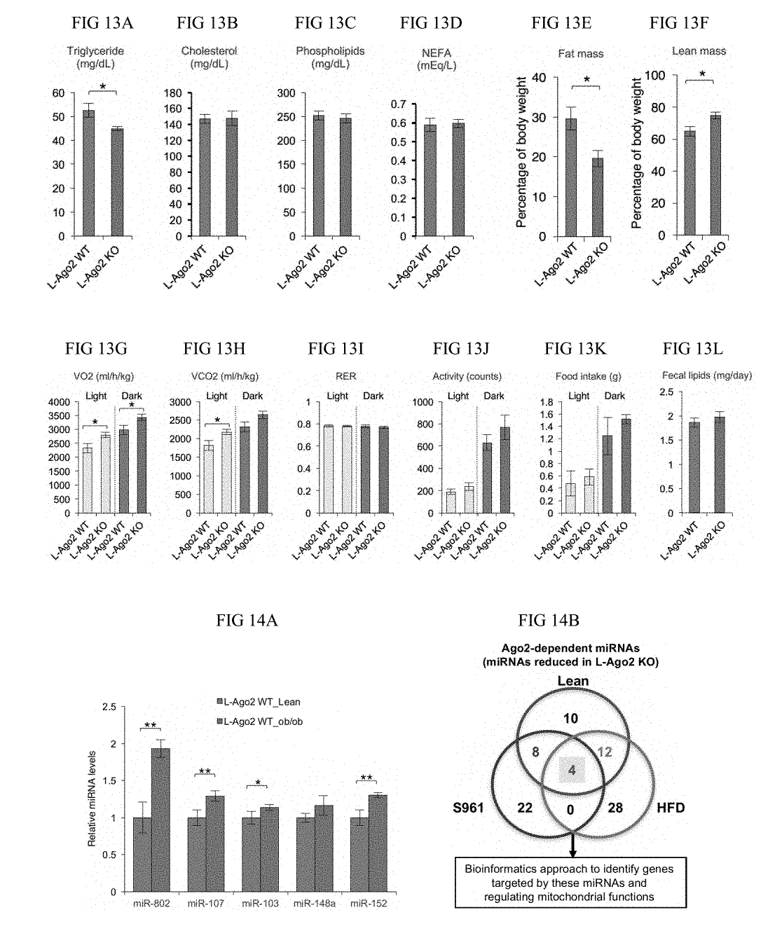

Importantly, the liver of L-Ago2 KO mice was characterized by lowered liver weights and triglyceride content, accompanied by lower serum ALT levels on HFD (FIG. 4I-4K). In addition, plasma triglyceride levels were lower in L-Ago2 KO mice fed HFD, while those of cholesterol, phospholipids, and free fatty acids were comparable between the genotypes (FIG. 13A-13D). There was also a reduction in hepatic fatty infiltration as visualized by haematoxylin and eosin (H&E) staining (FIG. 4L). While levels of genes involved in lipid biosynthesis such as Stearoyl-CoA desaturase-1 (Scd1) and Fatty acid synthease (Fasn) are comparable between the genotypes, those of Carnitine palmitoyltransferase 1A (Cpt1a) and Acetyl-coenzyme A synthetase 2-like (Acss1) that mediate catabolic processes of fatty acids are increased in the liver of L-Ago2 KO mice (FIG. 4M). Consistently, mitochondrial OCR in response to palmitate was significantly higher in Ago2-deficient hepatocytes compared to controls (FIG. 4N). Additionally, Applicant observed higher mitochondrial OCR in Ago2-deficient hepatocytes in the presence of acetate, which is one of the short-chain fatty acids (SCFAs) produced as a microbiota-fermentation product and whose circulating levels are positively correlated with obesity and its related sequelae³⁵ (FIG. 4N). These cellular phenotypes could promote reduction of hepatic triglyceride accumulation and lowered hepatic steatosis levels Applicant have observed in the liver of L-Ago2 KO mice fed HFD. Applicant further investigated if hepatic Ago2-deficency affected energy consumption. Quantitative Magnetic Resonance technology (EchoMRI) analyses revealed that total body fat mass in L-Ago2 KO mice is lower than in WT controls (44.3% fat reduction in L-Ago2 KO: p<0.05, t-test) (FIG. 13E), while lean mass composition of L-Ago2 KO mice was slightly higher than that of control mice on HFD at 20 weeks of age (FIG. 13F). In addition, the ability to utilize fat mass, which was calculated by measurements of fat and lean compositions before and after an overnight fast, was higher in L-Ago2 KO mice on HFD compared to WT controls (1.675-fold higher in L-Ago2 KO: p<0.01, t-test) (FIG. 4O). In support of these observations, there is an increased copy number of mitochondrial-DNA (mtDNA) in the Ago2-deficient liver in obesity (FIG. 4P). Consistently, rates of energy expenditure of L-Ago2 KO mice were significantly higher than those of controls (FIG. 4Q and FIGS. 13G and 13H), despite no significant changes in total physical activity, food intake, or amounts of fecal lipids between genotypes (FIG. 13I-13L). Applicant performed similar experiments with L-Ago1 WT and L-Ago1 KO mice fed HFD and found that there were no differences in the regulation of energy homeostasis between the genotypes (FIG. 11G-M). Taken together, these data indicate that inactivation of Ago2, but not Ago1, in the liver increases mitochondrial capacity and energy expenditure, which appears to link to improvement of obesity-associated pathophysiology.

Ago2-Mediated RNA Silencing Regulates Expression of Genes Involved in Energy Metabolism

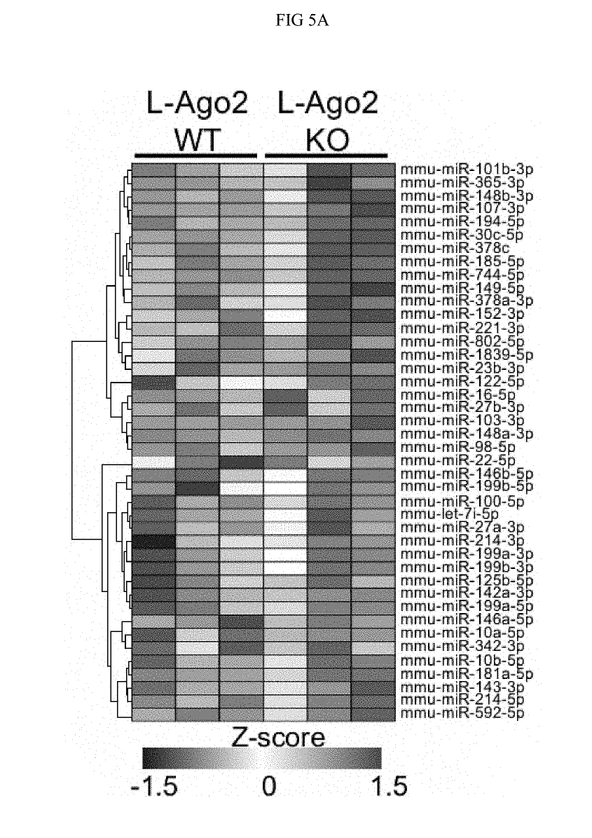

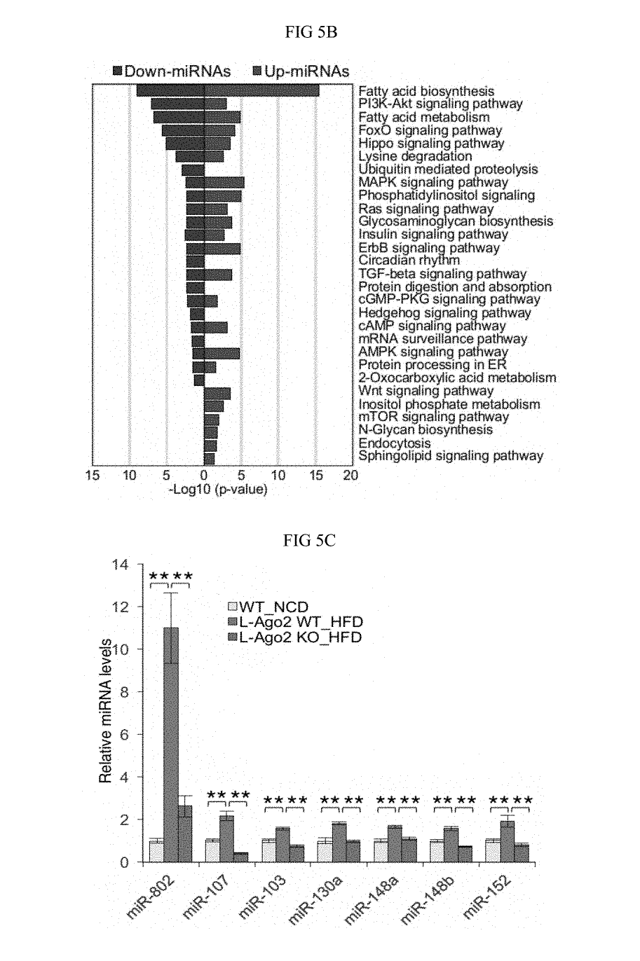

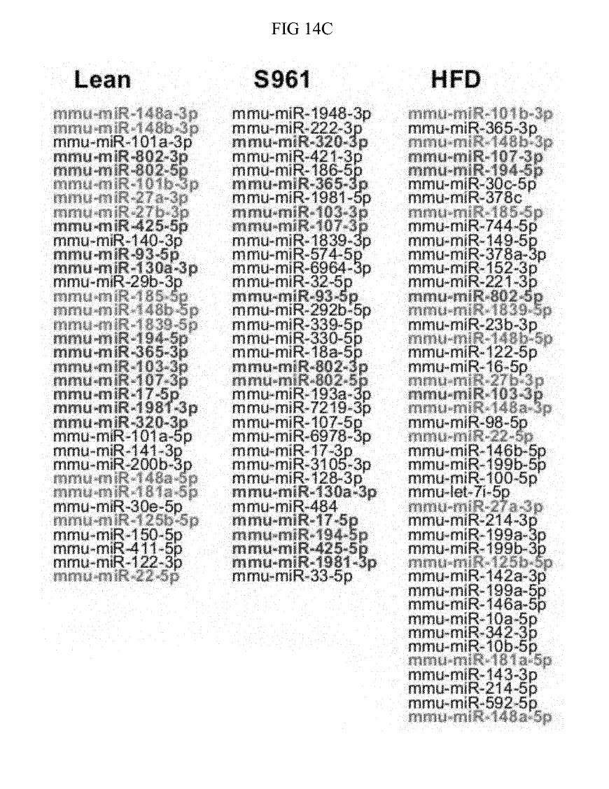

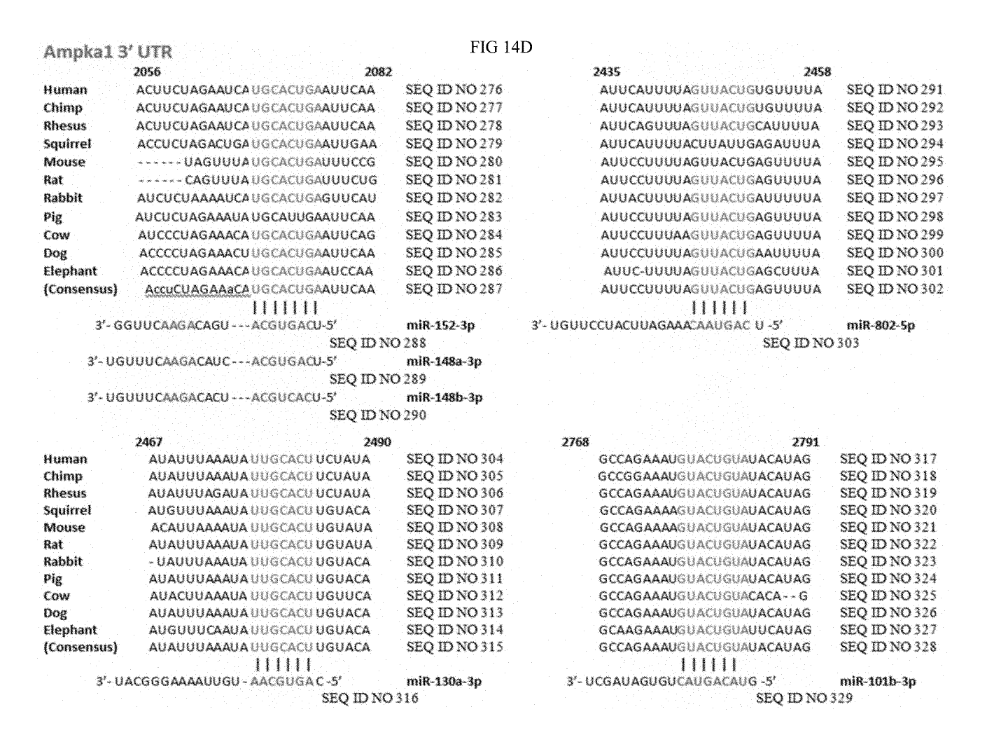

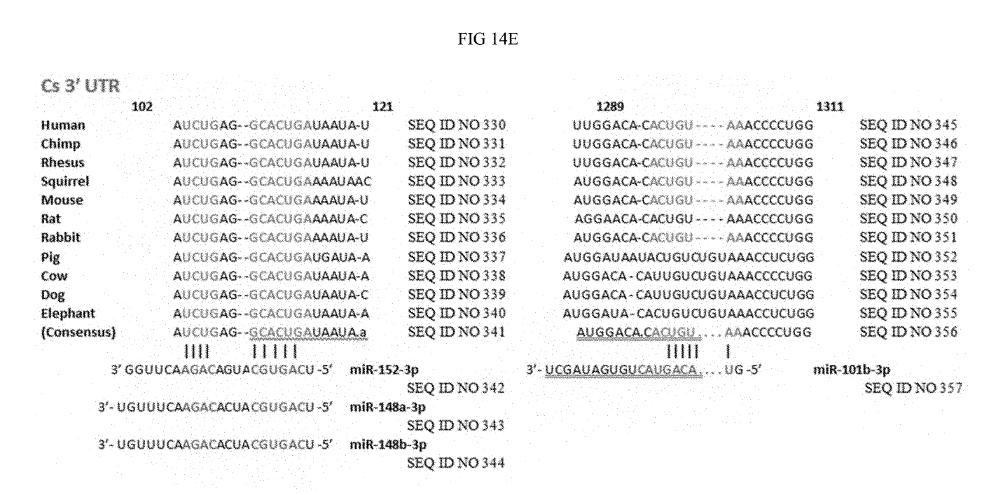

To investigate molecular mechanisms by which Ago2 orchestrates hepatic energy metabolism, Applicant additionally profiled hepatic miRNA expression under the condition of HFD (FIG. 5A). Utilizing the list of significant miRNAs on HFD, the miRNA target pathway enrichment analysis revealed that hepatic Ago2 deficiency affected several functional clades such as glucose and lipid metabolism and protein translation regulation (FIG. 5B). Importantly, while several MD-miRNAs were highly induced in the liver of L-Ago2 WT mice fed HFD and a leptin-deficient (ob/ob) obese mice (FIG. 5C and FIG. 14A), these miRNAs are constantly decreased in that of L-Ago2 KO mice (FIGS. 5A and 5C). These results further confirm the critical role of hepatic Ago2 in expressional regulation of a specific repertoire of MD-miRNAs known to deteriorate glucose and lipid metabolism in obesity. Consistent with these observations, expression levels of their known target mRNAs, such as Hnf1β, Cav1, and Pgc1α, are increased in L-Ago2 KO liver (FIG. 5D). Of note, expression levels of these genes were comparable between L-Ago1 WT and KO liver (FIG. 11N).

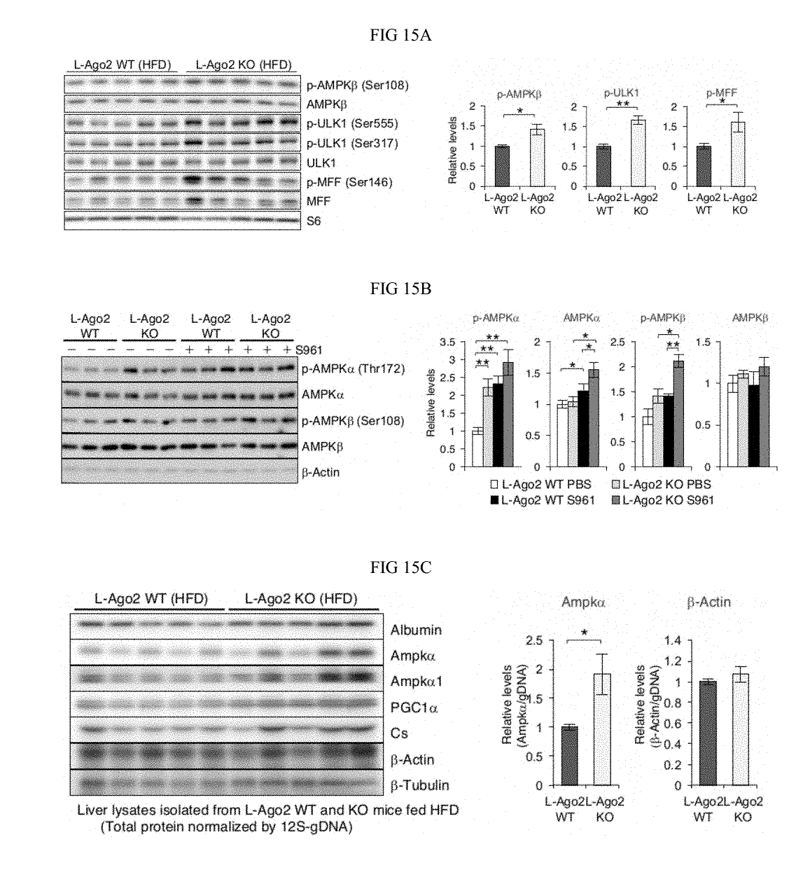

To explore targets of Ago2-dependent MD-miRNAs for metabolic regulation, Applicant then took a bioinformatics approach with miRNA sequencing data obtained under lean, HFD, and S961-treated conditions (FIGS. 15B and 14C). With the Ago2-dependent miRNAs, Applicant extracted lists of predicted conserved target genes involved in energy metabolism from the widely used TargetScan 7.1 website. This analysis identified a set of genes, including Ampka1 (also known as Prkaa1, a catalytic subunit of AMP-activated protein kinase (AMPK)) and Cs, that have 3′ untranslated region (UTR) containing multiple target sites for Ago2-dependent miRNAs including miR-148a/152 known to evoke hyperlipidemia, hypercholesteremia, and atherosclerosis^{14,15} (FIGS. 14D and 14E). As AMPK is known as a critical regulator of energy metabolism, Applicant further assessed the role of Ago2 in Ampka1 expression. Analyzing a public database of photoactivatable ribonucleoside-enhanced crosslinking and immunoprecipitation (PAR-CLIP) with Ago2³⁶ revealed that Ago2 binds to the region of the Ampka1 3′ UTR that contains binding sites of Ago2-dependent miRNAs including miR-148/152 and miR-130a (FIG. 5E). Consistent with these findings, Applicant confirmed the induction of Ampka1 expression, accompanied by the reduction of miR-148a, miR-148b, and miR-152, in the liver of L-Ago2 KO mice fed HFD and in Ago2-deficient primary hepatocytes (FIG. 5D and 5F). To further examine if miR-148/152 regulates Ampka1 expression, Applicant conducted luciferase assays in which Ampka1's 3′ UTR, with or without harboring a mutation at the miR-148/152 target site, was sub-cloned into luciferase expression vector. The luciferase activity of each was measured in primary hepatocytes isolated from L-Ago2 WT and KO mice (FIG. 5G). These analyses demonstrated that miR-148a/152 is involved in suppression of Ampka1 expression in a manner dependent on Ago2 and a miR-148a/152 target site (FIG. 5G). As miRNA inhibits the translation of target mRNAs through RNA silencing, Applicant additionally asked if Ago2-deficiency affects translation of genes having target sites of MD-miRNAs by investigating polysome-bound mRNA expression patterns. Expression levels of polysome-bound Ampka1 and Cs were enriched in Ago2-deficient primary hepatocytes, while those of β-Actin were comparable (FIG. 5H). Taken together, these findings further confirm that hepatic Ago2-mediated MD-miRNA biogenesis and RNA silencing are linked to expressional regulation of genes involved in energy metabolism (FIG. 5I).

Hepatic Ago2 Regulates Energy Consumption Associated with AMPK Activation

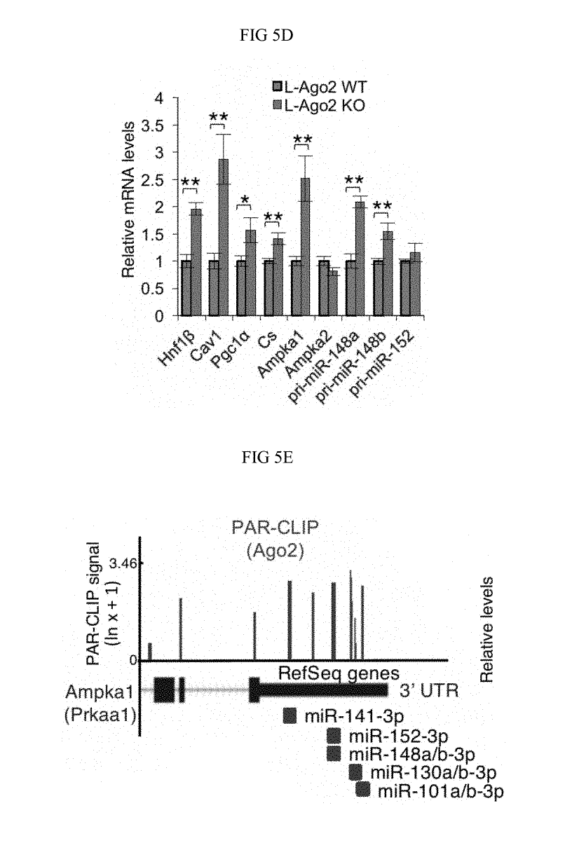

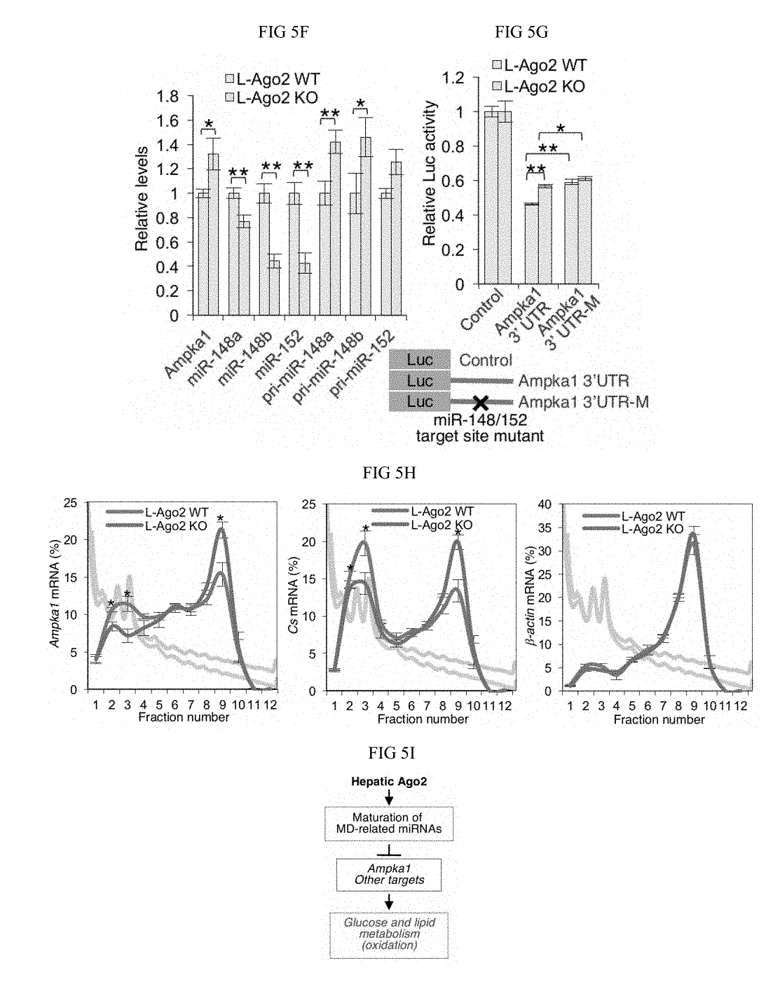

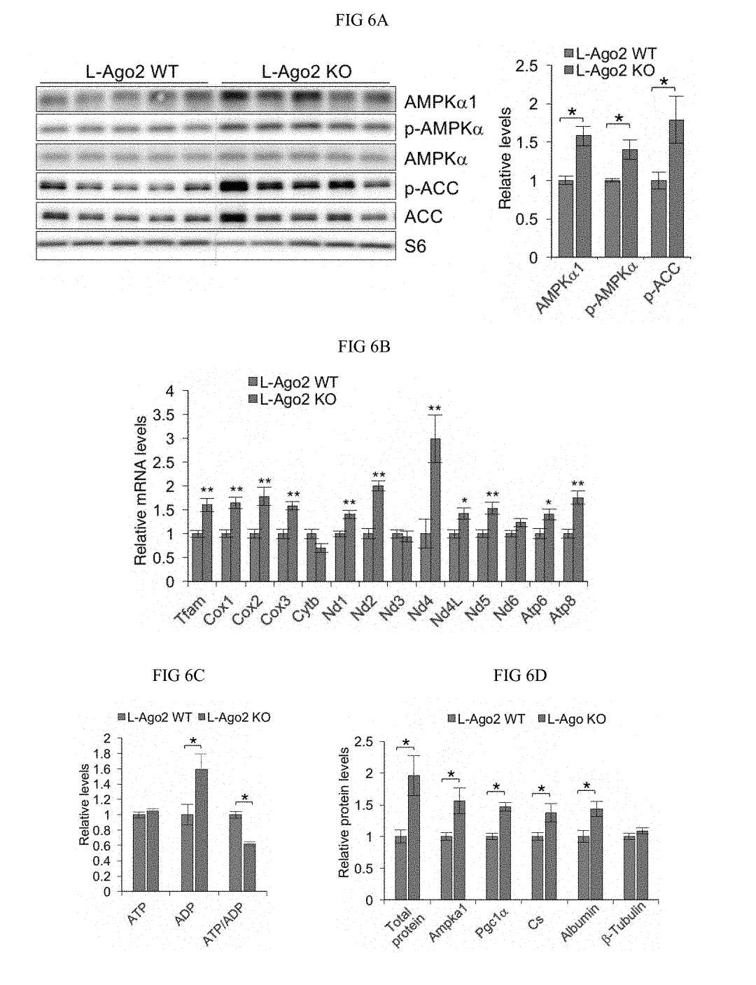

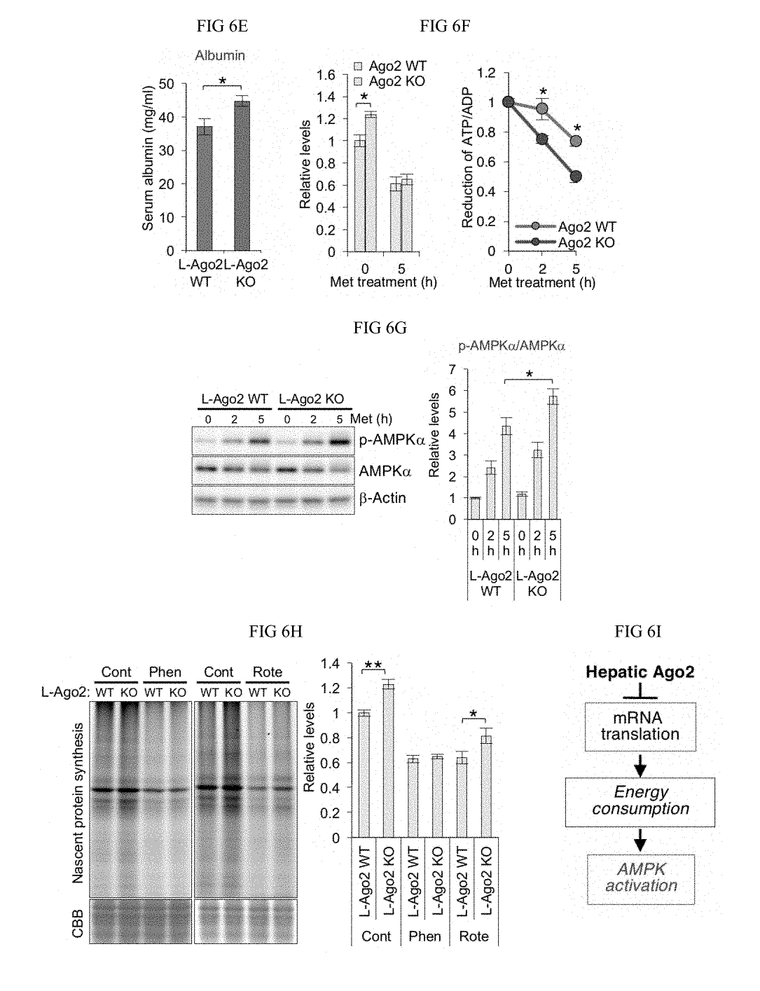

Given that Ago2 regulates translation of Ampka1 in hepatocytes, Applicant examined its protein levels and noticed that Ago2-deficiency increased not only protein levels of AMPKα1 but also activity of AMPK, assessed by phosphorylation levels of AMPKα, AMPKβ and an AMPK substrate, Acetyl-CoA carboxylase (ACC), in the liver of L-Ago2 KO mice fed HFD and treated with S961 (FIG. 6A and FIGS. 15A and 15B). Accumulating evidence demonstrates that AMPK is an energy sensor and that its activation enhances catabolic activities by stimulating mitochondrial biogenesis and autophagy/mitophagy that can improve the quality and quantity of mitochondria³⁷⁻³⁹. In addition, activation of the AMPK pathway improves glucose and lipid obesity³⁸ . Thus, Applicant further investigated the activation of AMPK and its substrates the liver of L-Ago2 WT and KO mice fed HFD. In agreement with the activation of AMPK, other AMPK substrates, UNC-51-like kinase 1 (ULK1), and Mitochondrial fission factor (MFF) are increased in the liver of L-Ago2 KO mice, suggesting enhanced autophagy/mitophagy and improved mitochondrial quality in the Ago2-deficient liver in obesity (FIG. 15A). Consistently, expression levels of the Tfam-mitochondrial gene pathway are increased in the liver of L-Ago2 KO mice fed HFD (FIG. 6B).

AMPK is activated when cellular energy level becomes low. Indeed, Applicant found a profound induction of ADP levels in the Ago2-deficient liver, while ATP levels are comparable between the genotypes, leading to a reduction of ATP/ADP ratio in the liver of L-Ago2 KO mice fed HFD (FIG. 6C). While Ago2-deficiency enhances capacity for mitochondrial oxidation and ATP production in hepatocytes (FIGS. 2G and 4K), systemic energy expenditure is also increased (FIG. 4N). Therefore, Applicant hypothesized that both energy production and consumption are enhanced in the liver of L-Ago2 KO mice compared to their controls. Since a main function of Ago2 is to suppress protein translation, which is one of the most energy consuming cellular processes, through RNA silencing, Applicant investigated the effect of Ago2-deficiency on protein synthesis in the liver. Levels of total and specific proteins normalized by DNA contents were higher in the liver of L-Ago2 KO mice (FIG. 6D and FIG. 15C). Similarly, examination of the levels of hepatic and serum albumin, one of the most abundant circulating proteins produced by the liver, revealed that the albumin levels were increased in L-Ago2 KO mice compared to their controls (FIGs. 6D and 6E). These observations suggest that enhanced protein synthesis in the liver may result in a lowered ATP/ADP ratio and AMPK activation in L-Ago2 KO mice.

To further examine if Ago2 deficiency accelerates cellular energy consumption associated with protein synthesis, Applicant treated primary hepatocytes with metformin, which is an anti-diabetic drug and inhibits mitochondrial respiratory-chain complex I activity restricting ATP generation⁴⁰, and measured ATP/ADP levels. Consistent with enhanced capacity for energy production in Ago2-deficiency (FIG. 2G), relative ATP/ADP levels were higher in Ago2-deficient hepatocytes compared to controls, however, the levels were rapidly decreased post-metformin treatment, suggesting a higher energy consumption rate in Ago2-deficiency (FIG. 6F). Consistently, levels of metformin-induced AMPK activation in Ago2-deficient hepatocytes were significantly higher than that in controls (FIG. 6G). To directly investigate the effect of Ago2-deficiency on protein synthesis in hepatocytes, Applicant measured the levels of nascent protein synthesis. Compared to WT controls, the levels were significantly increased in Ago2-deficient hepatocytes (FIG. 6H). By restricting energy supply using phenformin and rotenone, both of which inhibit mitochondrial respiratory-chain complex I activity, the levels of nascent protein synthesis in Ago2-deficient hepatocytes were equivalent or still higher compared to those in controls (FIG. 6G).

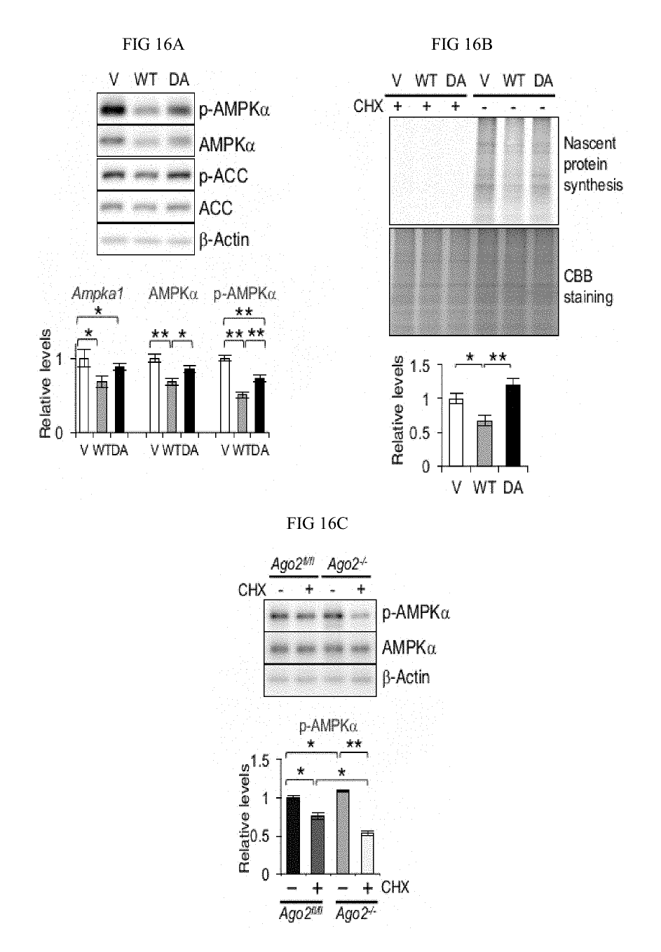

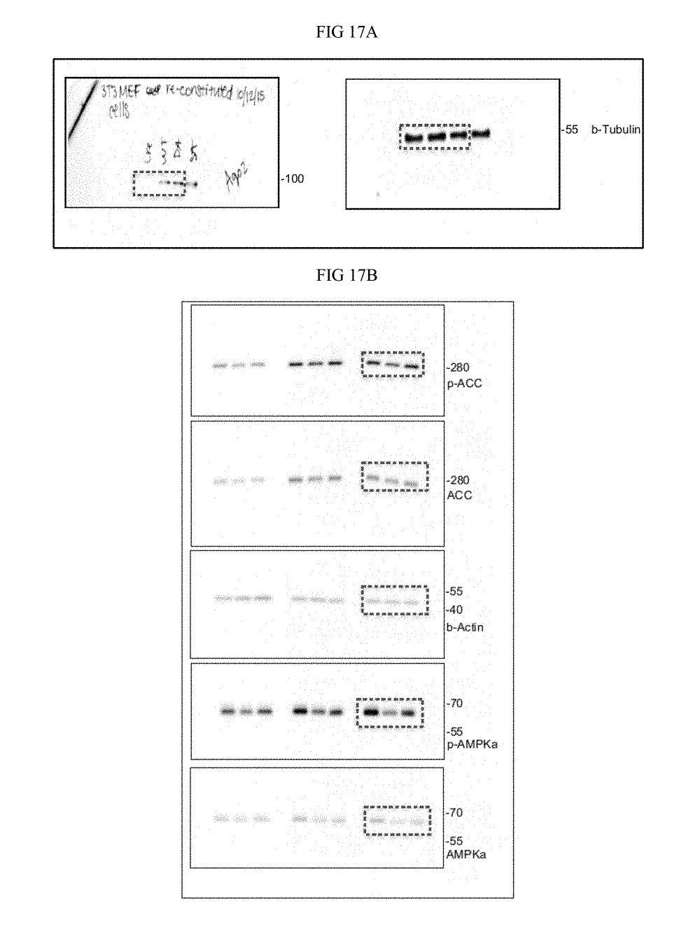

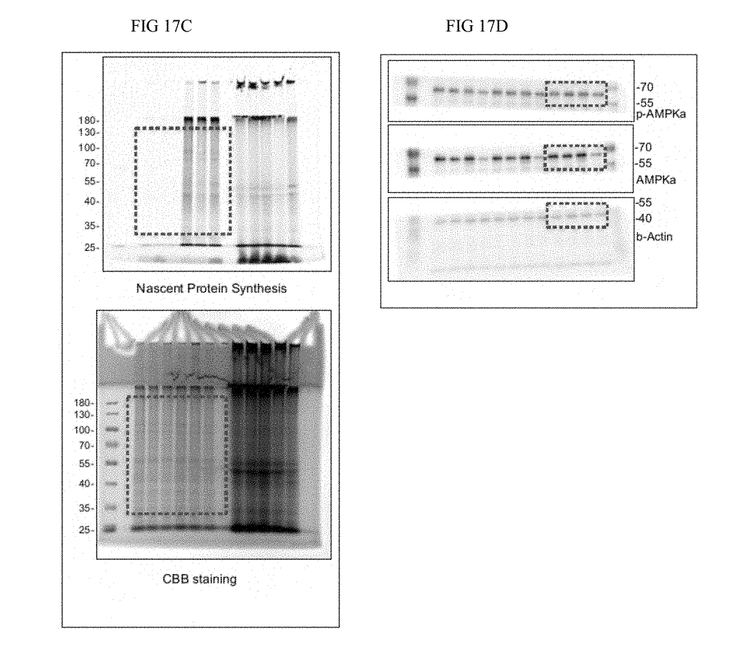

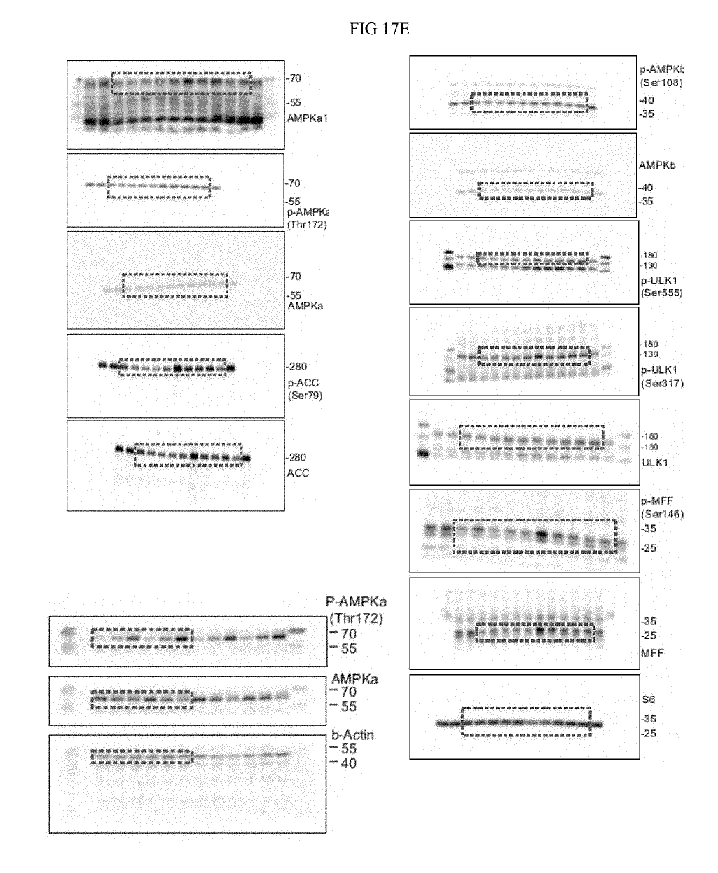

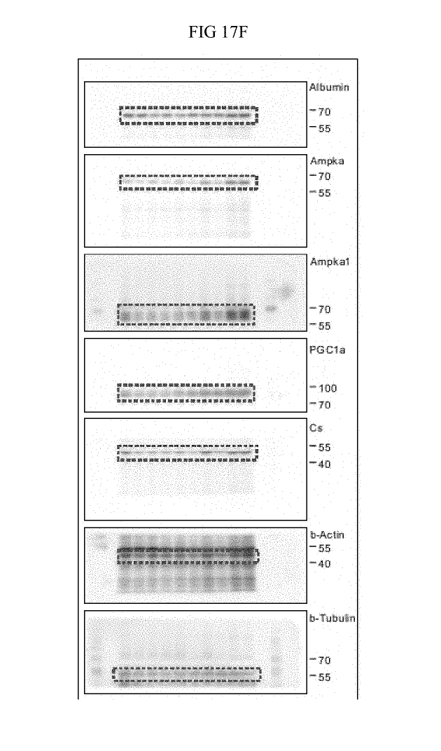

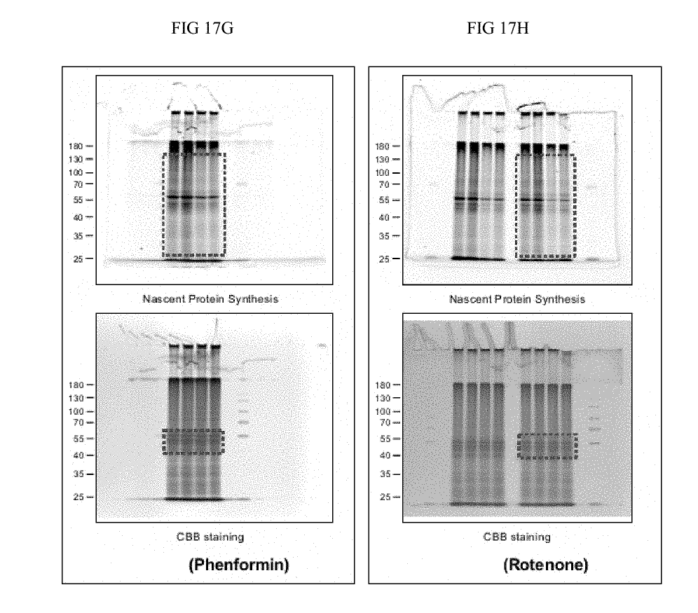

Since Ago2 slicer activity uniquely regulates RNA silencing, Applicant then asked if the slicer activity is involved in the regulation of energy consumption and AMPK activation. Ago2-deficient MEFs were characterized by enhanced expression of Ampka1, and reconstitution of the MEFs with WT Ago2 suppressed expression of both Ampka1 mRNA and AMPKα protein, whereas the Ago2 D669A mutant did not (FIG. 16A). More importantly, Applicant also observed that AMPK activity, assessed by phosphorylation levels of AMPKα and ACC, was higher in Ago2-deficient MEFs under serum starvation condition (FIG. 16A). These results indicate that Ago2 regulates both expression and activation of AMPK for which Ago2's slicer activity is crucial. While activated AMPKα is known to suppress mRNA translation⁴¹, levels of nascent protein synthesis in Ago2-deficient MEFs are increased compared to the cells reconstituted with WT Ago2 (FIG. 16B). To investigate if enhanced protein synthesis reasons AMPK activation in Ago2-deficiency, Applicant treated MEFs with cycloheximide (CHX), a protein synthesis inhibitor, and monitored AMPK activation. Inhibition of protein synthesis resulted in reduction of AMPK activation in WT MEFs, and the effect became more robust in Ago2-deficient MEFs, indicating that Ago2 suppresses protein synthesis-mediated energy consumption (FIG. 16C). Taken together, these results indicate that, in addition to an increase of Ampka1 expression, there is enhanced energy consumption associated with protein synthesis, leading to the lowered ATP/ADP ratio, which appears to enhance AMPK activation in Ago2-deficient conditions (FIG. 6I).

Hepatic Ago2-Deficiency Blunts Effects of Ampka1 Deletion and Metformin Treatment

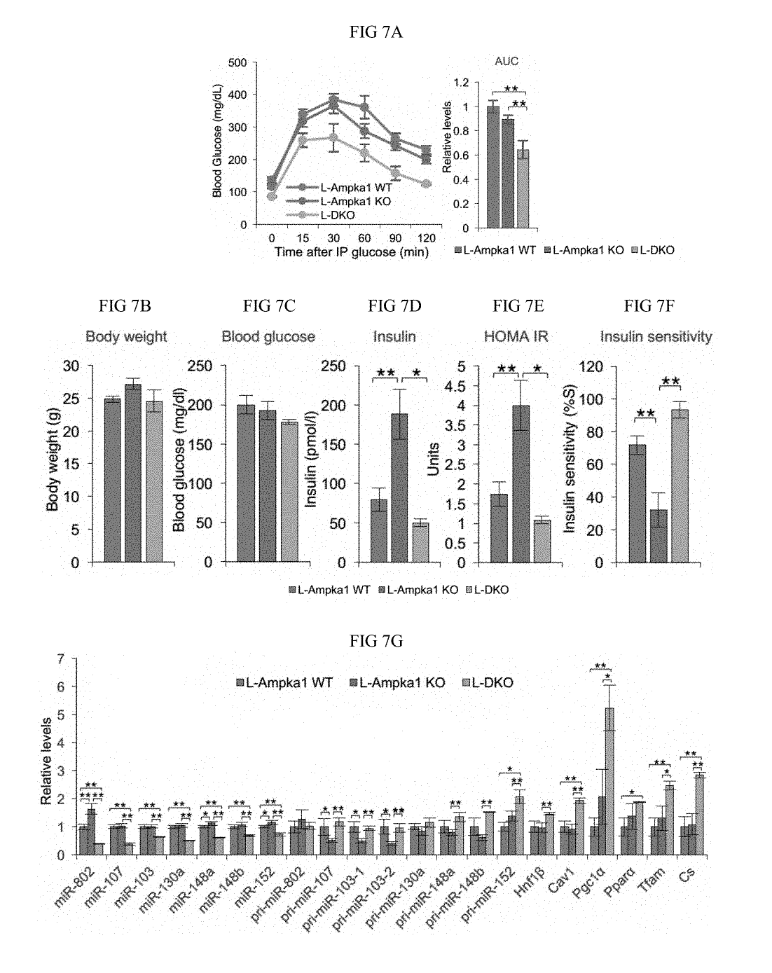

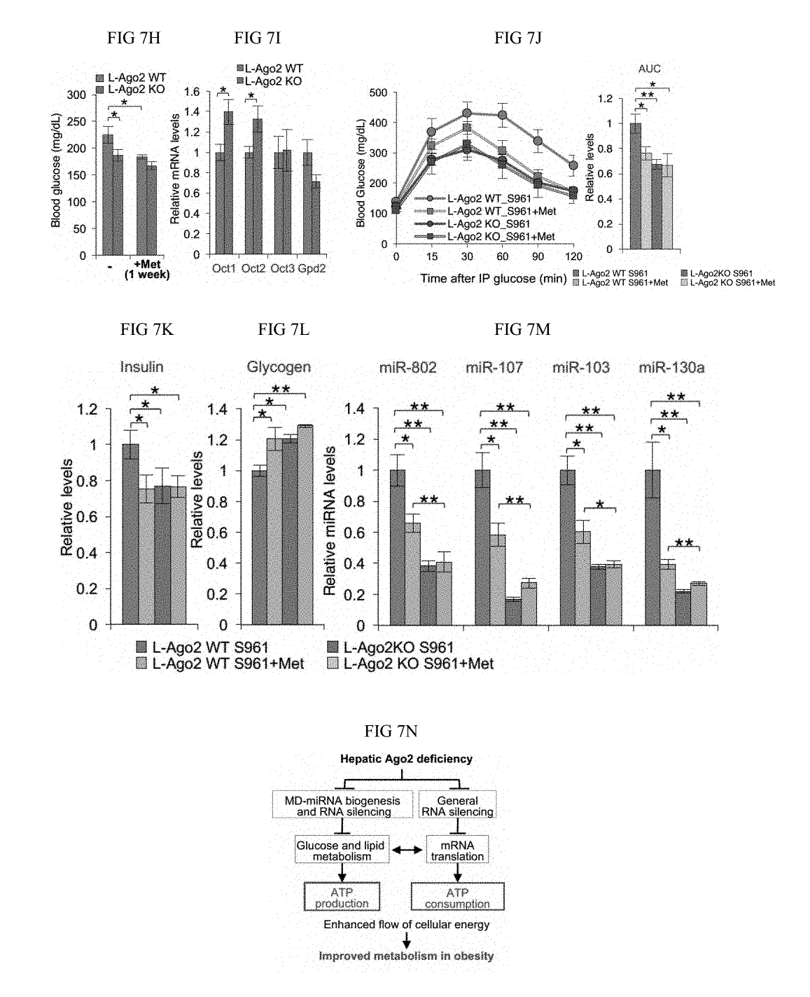

While hepatic Ago2-deficiency reduces biogenesis of a specific repertoire of MD-miRNAs of which some of them target Ampka1, it is obvious that changes in expression of these miRNAs also affects translation of other target mRNAs. Similarly, enhanced protein synthesis in the liver of L-Ago2 KO mice must influence not only AMPK activation but also other cellular events linked metabolic regulation. To clarify the role of Ampka1 in the metabolic alterations in L-Ago2 KO mice, Applicant generated liver-specific Ampka1- and Ago2-deficient mice (L-DKO mice) and placed them and their control groups, L-Ampka1 WT and L-Ampka1 KO mice, on HFD for analyses of glucose metabolism. While no significant difference was observed in body weight and fasting blood glucose levels among these three groups, L-DKO mice exhibited enhanced glucose tolerance in the condition of HFD feeding for 5 weeks (FIG. 7A-7C). Intriguingly, plasma insulin levels of L-Ampka1 KO mice were higher than those in L-Ampka1 WT mice and the levels were drastically decreased in L-DKO mice on HFD (FIG. 7D). These results indicate that inactivation of hepatic Ago2 can improve glucose metabolism even in an Ampka1-deficient condition where insulin resistance occurs (FIG. 7D-7F). Consistently, expression levels of a specific repertoire of MD-miRNA were constantly decreased in the liver of L-DKO mice, while levels of their targets, Hnf1β, Cav1, and Pgc1α and genes critical for enhancing mitochondrial function were higher in the liver of L-DKO mice compared to L-Ampka1 WT or L-Ampka1 KO mice (FIG. 7G). Taken together, these results suggest that the effect of hepatic Ago2-deficiency on glucose metabolism overrides that of AMPKα1 functions.

Applicant next investigated the glucose lowering effect of metformin in L-Ago2 KO mice. While there are distinct mechanisms of actions affecting mitochondrial functions between metformin treatment and Ago2-deficiency, both act toward changes in lowered cellular energy levels, AMPK activation, and improved glucose metabolism. Daily oral treatments of metformin for one week reduced fasting blood glucose levels in L-Ago2 WT mice fed HFD (FIG. 7H). Conversely, L-Ago2 KO mice without the treatment showed lower glucose levels compared to the controls, and these mice displayed no change upon metformin treatment (FIG. 7H). Of note, compared to control groups, expression levels of SLC22A1 and SLC22A3 (also known as Oct1 and Oct3, respectively), the main transporters responsible for metformin uptake, in the liver of L-Ago2 KO mice are higher or comparable (FIG. 7I). Applicant subsequently examined the effect of metformin treatment in L-Ago2 WT and KO mice treated with S961. While metformin treatment improved glucose tolerance in L-Ago2 WT mice, L-Ago2 KO mice showed better glucose tolerance without metformin, and showed no change in glucose tolerance with metformin (FIG. 7J). Consistently, while metformin alleviated S961-induced hyperinsulinemia and glycogenolysis in the WT control group, these effects were blunted in L-Ago2 KO mice (FIG. 7K and 7L). Importantly, metformin treatment resulted in drastic suppression of S961-induced MD-miRNAs in the liver of L-Ago2 WT mice, but did not affect their expression levels in Ago2-deficient liver (FIG. 7M). These results suggest that hepatic Ago2 deficiency mimics metformin's action in improving glucose metabolism and Ago2-dependent suppression of MD-miRNAs and RNA silencing may play an important role in metformin's beneficial effect.

Discussion

The role of RNA silencing in suppressing mRNA translation gives rise to the intriguing hypothesis that the RNA silencing machinery might be tightly integrated with the regulation of basal metabolic activity and energy homeostasis, as mRNA translation requires a massive amount of energy. However, to Applicant's knowledge, no metabolic or functional analysis has been carried out to test this concept. In this study, Applicant discovered that hepatic Ago2-mediated RNA silencing suppresses energy production in which Ago2 regulates biogenesis of specific miRNAs that silence genes critical for glucose and lipid metabolism, accompanied by reduced energy consumption linked to lowered mRNA translation. These findings suggest that Ago2's function is intimately linked to energy metabolism through regulating specific miRNA biogenesis and general mRNA translation, which balance energy production and consumption. Disruption of the hepatic Ago2-mediated energy balance in response to nutrient challenges appears to contribute to the pathogenesis of obesity-associated sequelae (FIG. 7N).

Although Ago1 and Ago2 share functional similarities in RNA silencing, Applicant's study provides evidence that Ago2 has a distinct role in metabolic regulation. Hepatic Ago1 is dispensable for obesity-induced pathophysiology, as deletion of hepatic Ago1 did not affect diet-induced weight gain, glucose tolerance, or insulin sensitivity. This, in turn, highlights the unique slicer activity of Ago2 in regulating the specific miRNA biogenesis and mRNA cleavage. While Ago2-deficiency in the liver affects expression of a small proportion of miRNAs, Applicant demonstrated that Ago2 plays a critical role in the maturation of a set of specific miRNAs, including miR-802, miR-103/107, and miR-148a/152, which are known to negatively impact glucose and lipid metabolism, for which Ago2's slicer activity is required. In addition, expression of these miRNAs is enhanced in response to the energy stress conditions of lower insulin availability or sensitivity, in an Ago2-dependent manner Under these stress conditions, hepatocytes are normally programmed to stimulate glucose production, triglyceride synthesis, and the assembly and secretion of very low-density lipoprotein particles. Hepatic Ago2 is likely integrated into this program through the generation of selective miRNAs, and by mediating subsequent RNA silencing. While this mechanism may be beneficial for the maintenance of systemic energy homeostasis during hypoglycemia, hypermotility, starvation, and developmental processes, it could also accelerate the development of metabolic diseases in excess nutrient conditions. When each of the four Ago proteins are ablated constitutively in mice, only the loss of Ago2 causes embryonic lethality, whereas loss of other three Ago proteins is dispensable for animal development^{24,42-45.} Importantly, Ago2's slicer activity is required for embryonic and perinatal development'. Of note, while Applicant highlighted the novel role of Ago2's slicer activity in the biogenesis of specific MD-miRNAs, the activity is evidently involved in mRNA cleavage. It is reasonable to consider a model where Ago2's unique function regulates energy metabolism not only in the liver but also in other organs during development and in adulthood. This may, at least in part, explain the universal importance of Ago2 in such a diverse array of mammalian organs.

One important question is how Ago2's slicer activity is regulated in response to metabolic challenge. The possibility that Ago2's slicer activity might function as part of a signaling node for stress responses to alter the cell's program of RNA silencing should be considered. Intriguingly, a recent study showed that hypoxia reduces the binding of Dicer with Ago2, thus inhibiting the processing of precursor miRNAs to mature miRNAs⁴⁶. Furthermore, protein kinase B gamma (Akt3) was shown to inhibit Ago2's slicer activity by phosphorylating Ago2 at serine 387⁴⁷. These findings suggest that Ago2 possesses capability to respond to changes in cellular conditions and to control the RNA silencing output and metabolic consequences. Consistently, the most attractive implication of the observation disclosed herein is that Ago2 is responsible for induction of a set of MD-miRNAs and silencing genes regulating energy metabolism during metabolically-driven stress conditions. While there is a specific miRNA, miR-451, known to be generated through a non-canonical pathway, which bypasses Dicer and exclusively requires the slicer activity of Ago2^{21,22,48}, Applicant's work suggests that the contribution of additional miRNAs processed through the non-canonical pathway in different cell types and cellular stress conditions should be also considered.

Applicant has demonstrated that Ago2-mediated RNA silencing connects the regulation of energy supply with protein biosynthesis. This mechanism may be the core of a vicious cycle in disrupted energy metabolism in the obese liver. In this setting, despite a robust activation of the mTORC1 pathway, protein biosynthesis is progressively suppressed, which is a paradox of mRNA translation^{6,49}. While obesity is traditionally considered a state of over-nutrition, recent studies suggest that the obese liver may, in some aspects, resemble a condition of energy deprivation in which proper catabolic processes are impaired due to the repression of oxidative phosphorylation pathways and mitochondrial gene expression^{7,50}. Consistently, obesity is also known to induce defects in autophagy in the liver, which leads to poor mitochondrial quality control⁵¹⁻⁵³ As a result, protein biosynthesis may be impaired due to under-powered energy supply even during the activation of the mTORC1 pathway, leading to further accumulation of energy sources. Conversely, hepatic Ago2-deficiency increases expression of key metabolic genes including Ampka1 with enhanced cellular energy consumption that can lead to lower ATP/ADP ratio. This condition can amplify activation of AMPK and its substrates ULK1, MFF, and Pgc1α, leading to improved mitochondrial capacity and quality, which in turn generates sufficient energy for protein biosynthesis. Of note, Ago2 is also known to regulate mRNA silencing through interacting with exonuclease complexes, the Ccr4-Not and Pan2-Pan3 complexes⁵⁴, in a miRNA-independent manner, which likely contributes to suppression of protein translation and energy metabolism in the liver. These Ago2-mediated molecular events may solve the paradox of protein biosynthesis in the obese liver, demonstrate a new mechanism in the regulation of basal metabolic activity, and provide a novel therapeutic target for metabolic diseases.

While metformin's molecular mechanism of action in improving glucose metabolism has remained enigmatic, it is generally accepted that actions of metformin on mitochondria underlie most of the pleiotropic effects of the drug in its primary target tissue, the liver⁴⁰. As such, metformin inhibits the mitochondrial respiratory-chain complex I, resulting in a drop in cellular ATP concentration and activation of AMPK, although the AMPK activation appears to be dispensable for improving glucose metabolism in metformin's action⁴⁰. Of note, a recent study has shown that metformin inhibits hepatic protein synthesis through its dose-dependent mechanism, although it remains unclear if the suppressed protein synthesis is involved in metformin's glucose lowering effect⁵⁵. Conversely, Ago2 deficiency enhances both mitochondrial oxidation and protein synthesis, which could change the status of cellular energy balance at different nutrient conditions and activate the AMPK pathways. Despite the distinct mechanisms affecting energy metabolism, especially an opposite effect on protein synthesis between metformin's action and Ago2 deficiency, metformin's effect is, at least in part, blunted in mouse models of hepatic Ago2-deficiency. Importantly, metformin is reported to cause drastic changes in miRNA expression profiles⁵⁶, and Applicant's study reveals the role of Ago2 in the effect of metformin on MD-miRNA expression (FIG. 7M). These findings raise the possibility that changes in the specific miRNA-RNA networks regulated by Ago2 may be key in metformin's action in the regulation of glucose metabolism. Comprehensive analyses of the miRNA-RNA networks in metformin's action might provide fundamental insight into the regulation of energy metabolism associated with mitochondrial functions and protein synthesis and lead to novel therapeutic approaches for hyperglycemia.

In conclusion, Ago2 uniquely regulates energy production and consumption in the liver, and suggest hepatic Ago2-mediated RNA silencing is a core regulator of energy metabolism during the pathogenesis of obesity. Thus, Ago2 may be a potential target for therapeutic interventions for modulation of a spectrum of Ago2-dependent miRNA-mediated events, in chronic metabolic disorders, such as diabetes, fatty liver diseases, and other obesity-associated sequelae.

Materials and Methods

Mice. Animal care and experimental procedures were performed according to procedures approved by the animal care committees of our medical center. Ago1fl/fl, Ago2^{fl/fl}, and Albumin^{cre/cre} were obtained from the Jackson Laboratory (Stock No: 019001, 016520, and 003574, respectively). Ampka1^{fl/fl} mice were kindly provided by Dr. Basilia Zingarelli. All mice used in this study were on C57BL/6 background. Mice were placed on a high-fat diet (HFD:60% fat, 20% protein, and 20% carbohydrate kcal; Research Diets #D12492) for a diet induced obesity model, a control diet (CD: 10% fat, 20% protein, and 70% carbohydrate; Research Diets #D12450), or normal chow diet (NCD: 29% Protein, 13% Fat and 58% Carbohydrate kcal; LAB Diet #5010) beginning at 4 weeks of age ad libitum with free access to water. For acute insulin resistant model, S961, an insulin receptor antagonist, was kindly provided by Dr. Lauge Schaffer'. The ALZET osmotic pump were used to deliver 10 nM S961 or vehicle (PBS) in a two-week period. GTTs were performed by intraperitoneal glucose injection (1.5 g/kg) following an overnight food withdrawal for 14 hours. ITTs were performed by intraperitoneal insulin injection (0.75 IU/kg for lean mice, 1 IU/kg for obese mice) following a daytime food withdrawal for 6 hours. PTTs were performed by intraperitoneal sodium pyruvate injection (Sigma-Aldrich, 2 g/kg) following an overnight food withdrawal for 16 hours. Body composition was analyzed by EchoMRI^{TM}-100H instrument (Echo Medical Systems) as previously described⁵⁷. To measure body composition after fasting, food was removed from mice for 16 hours. To analyze fecal lipid excretion, lipid content of feces was extracted using chloroform:methanol (2:1) and air-dried under a fume hood. Mouse serum albumin levels were measured using an ELISA kit (Abcam). Mouse plasma insulin levels were measured using Mouse Ultrasensitive Insulin ELISA kit (ALPCO). Lipid profiling was also performed by University of Cincinnati's MMPC Core. Energy expenditure was measured by using PhenoMaster (TSE Systems) as previously described⁵⁸. Hyperinsulinemic-euglycemic clamp studies were performed at University of Michigan Animal Phenotyping Core.

Biochemical reagents and antibodies. All biochemical reagents were purchased from Sigma-Aldrich unless otherwise indicated. Antibodies against, JNK1 (SC-1648, 1:2,500), Dicer (SC-592 30226, 1:2,500), Akt (SC-8312, 1:2,500), phospho-Akt (Ser473) (SC-7985-R, 1:2,500), PGC-1α 593 (SC-13067, 1:2,500), β-actin (SC-130656, 1:5,000), and β-tubulin (SC-9104, 1:5,000) were from Santa Cruz Biotechnology. Anti-Acc (3662, 1:2,500), anti-phospho-Acc (Ser79) (11818, 1:2,500), Anti-AMPKα (5832, 1:2,500), Anti-phospho-AMPKα (Thr172) (2535, 1:2,500), anti-AMPKβ (4250, 1:2,500), anti-phospho-AMPKβ (Ser108) (4181, 1:2,500), anti-phospho-ULK1 (Ser555) (5869, 1:2,500), anti-phospho-ULK1 (Ser317) (12753, 1:2,500), anti-ULK1 (8054, 1:2,500), anti-phospho-MFF (Ser146) (49281, 1:2,500), anti-MFF (86668, 1:2,500), anti-Ago2 (2897, 1:2,500), anti-Ago1 (5053, 1:2,500), anti-S6 Ribosomal Protein (2217, 1:2,500) and anti-phospho-JNK (Thr183/Tyr185) (1:2,500), anti-Albumin (4929, 1:2,500), anti-Citrate Synthase (14309, 1:2,500), anti-AMPKα1 (2795, 1:2,500) were purchased from Cell Signaling Technology. Anti-IRS1 (1:2,500) and anti-phospho-IRS (Ser307) (07247, 1:2,500) antibodies were purchased from Upstate Biotechnology. Anti-AMPKα1 (32047, 1:2,500) was purchased from Abcam

Primary hepatocytes. Hepatocytes were isolated from liver of 12-14 weeks old L-Ago2 WT and 607 L-Ago2 KO mice by a two-step perfusion method as described previously⁵⁹ with a slight modification. Briefly, the liver was first perfused with 30 ml of HBSS supplemented with 10 mM HEPES, 0.5 mM EGTA and 5 mM glucose and then digested with 35 mL of Collagenase X 610 (WAKO) at 100 U/mL dissolved in HBSS buffer supplemented with 10 mM HEPES and 5 mM CaCl₂. Liver was collected after perfusion and hepatocyte were released and sedimented at 60 G for 2 mM. Hepatocyte suspension was then layered on a 40% percoll solution (GE Healthcare Life Sciences) and centrifuged at 800 G for 10 min. The alive hepatocytes were recovered from the bottom of the tube and seeded on culture plates.

Mouse embryonic fibroblasts. Ago2-deficient fibroblasts reconstituted with Ago2 WT or DA mutant that were kindly provided by Dr. Eric Lai²². Applicant generated Ago2^{fl/fl} MEFs through the 3T3 protocol and performed an adenovirus-mediated gene transfer for Cre or LacZ expression to obtain Ago2-deficient MEFs⁶⁰. MEF cells were cultured in Dulbecco Modified Eagle Medium (DMEM) (Thermo Fisher Scientific: #11965) supplemented with 10% FBS. For western blot analyses of AMPK, MEF cells were plated at a density of 1×10⁵ cells per well of 6-well plate for overnight. Next day, cells were kept under serum starvation condition in glucose-, pyruvate-, and glutamine-free DEME (Thermo Fisher Scientific: #A1443001).

Quantitative real-time PCR analysis. For mRNA quantification, total RNA was extracted using Trizol reagent (Invitrogen). Total RNA was converted to first strand cDNA using SuperScript® VILO™ cDNA Synthesis Kit (Invitrogen). Quantitative real-time PCR analysis was performed using SYBR Select Master Mix (Applied Biosystems) in a real-time PCR machine (QuantStudio 6 Flex Real-Time PCR system; Thermo Fisher Scientific). Primers are listed in Table 2. To normalize expression data, β-actin mRNA was used as a housekeeping gene. For miRNA quantification, total RNA was extracted using miRNeasy Micro Kit (Qiagen) according to manufacturer's instructions. TaqMan miRNA assays (Life Technologies) were used and real-time PCR were carried out for mature miRNA quantification. Primary miRNAs were quantified using TaqMan Fri-miRNA assays. Sno202 and β-actin were used as internal controls.

TABLE 2
List of primers used in this study.

| Gene | Species | Accession # | | Forward | | | Reverse | | |
|---|---|---|---|---|---|---|---|---|---|
| mRNA | | | | | | | | | |
| β-actin | Mouse | NM_007393 | 5′- | GGC TGT | -3′ | 5′- | CCA GTT | -3′ | SEQ ID NO |
| | | | | ATT CCC | | | GGT AAC | | |
| | | | | CTC CAT | | | AAT GCC | | |
| | | | | CG | | | ATG T | | |
| G6Pase | Mouse | NM_008061 | 5′- | CGA CTC | -3′ | 5′- | GTT GAA | -3′ | SEQ ID NO |
| | | | | GCT ATC | | | CCA GTC | | |
| | | | | TCC AAG | | | TCC GAC | | |
| | | | | TGA | | | CA | | |
| Ppar-γ | Mouse | NM_011146.3 | 5′- | GCA TGG | -3′ | 5′- | TGG CAT | -3′ | SEQ ID NO |
| | | | | TGC CTT | | | CTC TGT | | |
| | | | | CGC TGA | | | GTC AAC | | |
| | | | | | | | CAT G | | |
| Ppar-α | Mouse | NM_001113418.1 | 5′- | TGT TTG | -3′ | 5′- | GCA ACT | -3′ | SEQ ID NO |
| | | | | TGG CTG | | | TCT CAA | | |
| | | | | CTA TAA | | | TGT AGC | | |
| | | | | TTT GC | | | CTA TGT | | |
| | | | | | | | TT | | |
| Tfam | Mouse | NM_009360.4 | 5′- | CAC CCA | -3′ | 5′- | CTG CTC | -3′ | SEQ ID NO |
| | | | | GAT GCA | | | TTT ATA | | |
| | | | | AAA CTT | | | CTT GCT | | |
| | | | | TCA G | | | CAC AG | | |
| Cs | Mouse | NM_026444.3 | 5′- | GGG ACT | -3′ | 5′- | AGC CAA | -3′ | SEQ ID NO |
| | | | | TGT GTA | | | AAT AAG | | |
| | | | | TGA GAC | | | CCC TCA | | |
| | | | | TTC G | | | GG | | |
| Cytc | Mouse | NM_007808.4 | 5′- | GGA GGC | -3′ | 5′- | TCC ATC | -3′ | SEQ ID NO |
| | | | | AAG CAT | | | AGG GTA | | |
| | | | | AAG ACT | | | TCC TCT | | |
| | | | | GG | | | CC | | |
| mt-Cox1 | Mouse | NC_005089.1 | 5′- | TCC AAC | -3′ | 5′- | TCC TGC | -3′ | SEQ ID NO |
| | | (5328-6872) | | TCA TCC | | | TAT GAT | | |
| | | | | CTT GAC | | | AGC AAA | | |
| | | | | ATC | | | CAC T | | |
| mt-Cox2 | Mouse | NC_005089.1 | 5′- | CTA ATT | -3′ | 5′- | TTC GTA | -3′ | SEQ ID NO |
| | | (7013-7696) | | AGC TCC | | | GCT TCA | | |
| | | | | TTA GTC | | | GTA TCA | | |
| | | | | CTC | | | TTG | | |
| mt-Cox3 | Mouse | NC_005089.1 | 5′- | ATT CTA | -3′ | 5′- | AAG GCT | -3′ | SEQ ID NO |
| | | (8607-9390) | | TTC ATC | | | ATG ATG | | |
| | | | | GTC TCG | | | AGC TCA | | |
| | | | | GAA | | | TGT | | |
| mt-Atp6 | Mouse | NC_005089.1 | 5′- | TAA TCA | -3′ | 5′- | GTG TCG | -3′ | SEQ ID NO |
| | | (7927-8607) | | ACA ACC | | | GAA GCC | | |
| | | | | GTC TCC | | | TGT AAT | | |
| | | | | ATT C | | | TAC | | |
| mt-Atp8 | Mouse | NC_005089.1 | 5′- | TGC CAC | -3′ | 5′- | GGT AAT | -3′ | SEQ ID NO |
| | | (7766-7969) | | AAC TAG | | | GAA TGA | | |
| | | | | ATA CAT | | | GGC AAA | | |
| | | | | CAA | | | TAG | | |
| mt-Cytb | Mouse | NC_005089.1 | 5′- | GCA ACG | -3′ | 5′- | TGA GAT | -3′ | SEQ ID NO |
| | | (14145-15288) | | AAG CCT | | | TGG TAT | | |
| | | | | AAT ATT | | | AAG AAT | | |
| | | | | CC | | | TAA | | |
| mt-Nd1 | Mouse | NC_005089.1 | 5′- | TTA CCA | -3′ | 5′- | ATC GTA | -3′ | SEQ ID NO |
| | | (2751-3707) | | GAA CTC | | | ACG GAA | | |
| | | | | TAC TCA | | | GCG TGG | | |
| | | | | ACT | | | ATA | | |
| mt-Nd2 | Mouse | NC_005089.1 | 5′- | TCA ATA | -3′ | 5′- | ATG ATA | -3′ | SEQ ID NO |
| | | (3914-4951) | | ATT ATC | | | GTA GAG | | |
| | | | | CTC CTG | | | TTG AGT | | |
| | | | | GCC | | | AGC | | |
| mt-Nd3 | Mouse | NC_005089.1 | 5′- | TTC TAG | -3′ | 5′- | ATA GAA | -3′ | SEQ ID NO |
| | | (9459-9806) | | TTG CAT | | | TTG TGA | | |
| | | | | TCT GAC | | | CTA GAA | | |
| | | | | TCC | | | TAA | | |
| mt-Nd4 | Mouse | NC_005089.1 | 5′- | GCC TGA | -3′ | 5′- | GGT TCC | -3′ | SEQ ID NO |
| | | (10167-11544) | | TTA CTG | | | CTC ATC | | |
| | | | | CCA CTA | | | GGG TAA | | |
| | | | | ATA | | | TAA | | |
| mt-Nd4L | Mouse | NC_005089.1 | 5′- | ACT ATC | -3′ | 5′- | TTG GAC | -3′ | SEQ ID NO |
| | | (9877-10173) | | ACT TCT | | | GTA ATC | | |
| | | | | AGG GAC | | | TGT TCC | | |
| | | | | ACT | | | GT | | |
| mt-Nd5 | Mouse | NC_005089.1 | 5′- | AAC CAC | -3′ | 5′- | CAG GCG | -3′ | SEQ ID NO |
| | | (11742-13565) | | ACC TAG | | | TTG GTG | | |
| | | | | CAT TCC | | | TTG CAG | | |
| | | | | TAC | | | GTA | | |
| mt-Nd6 | Mouse | NC_005089.1 | 5′- | ACA ACT | -3′ | 5′- | GAT ATA | -3′ | SEQ ID NO |
| | | (13552-14070, | | ATA TAT | | | CGA CTG | | |
| | | complement) | | TGC CGC | | | CTA TAG | | |
| | | | | TAC | | | CTA | | |
| Oct1 | Mouse | NM_009202.5 | 5′- | GAC GCC | -3′ | 5′- | GCA ACA | -3′ | SEQ ID NO |
| | | | | TGG AAA | | | TGG ATG | | |
| | | | | GTG GAC | | | TAT AGT | | |
| | | | | C | | | CTG GG | | |
| Oct3 | Mouse | NM_011395 | 5′- | AGC CAG | -3′ | 5′- | TGA GCT | -3′ | SEQ ID NO |
| | | | | CCC GAC | | | CTG AGC | | |
| | | | | TAC TAT | | | TGG TAT | | |
| | | | | TGG T | | | TAG T | | |
| Gpd2 | Mouse | NM_001145820 | 5′- | GAA GGG | -3′ | 5′- | GGA TGT | -3′ | SEQ ID NO |
| | | | | GAC TAT | | | CAA ATT | | |
| | | | | TCT TGT | | | CGG GTG | | |
| | | | | GGG T | | | TGT | | |
| Cav-1 | Mouse | AB029929 | 5′- | ATG TCT | -3′ | 5′- | CGC GTC | -3′ | SEQ ID NO |
| | | | | GGG GGC | | | ATA CAC | | |
| | | | | AAA TAC | | | TTG CTT | | |
| | | | | GTG | | | CT | | |
| Hnf1β | Mouse | NM_009330.3 | 5′- | CAC CAA | -3′ | 5′- | GGA GTG | -3′ | SEQ ID NO |
| | | | | GCC GGT | | | TCA TAG | | |
| | | | | TTT CCA | | | TCG TCG | | |
| | | | | TAC | | | CC | | |
| Pgc1a | Mouse | NM_008904 | 5′- | CAG CCT | -3′ | 5′- | CCG CTA | -3′ | SEQ ID NO |
| | | | | CTT TGC | | | GCA AGT | | |
| | | | | CCA GAT | | | TTG CCT | | |
| | | | | CT | | | CA | | |
| Tnfa | Mouse | NM_013693.3 | 5′- | GCT ACG | -3′ | 5′- | CCC TCA | -3′ | SEQ ID NO |
| | | | | ACG TGG | | | CAC TCA | | |
| | | | | GCT ACA | | | GAT CAT | | |
| | | | | G | | | CTT CT | | |
| Srebp1c | Mouse | NM_011480.4 | 5′- | GGA GCC | -3′ | 5′- | GCT TCC | -3′ | SEQ ID NO |
| | | | | ATG GAT | | | AGA GAG | | |
| | | | | TGC ACA | | | GAG GCC | | |
| | | | | TT | | | AG | | |
| Scd1 | Mouse | NM_009127.4 | 5′- | CGG GAT | -3′ | 5′- | TTC TTG | -3′ | SEQ ID NO |
| | | | | TGA ATG | | | CGA TAC | | |
| | | | | TTC TTG | | | ACT CTG | | |
| | | | | TCG T | | | GTG C | | |
| Fasn | Mouse | NM_007988.3 | 5′- | AAG GCT | -3′ | 5′- | GGA GTG | -3′ | SEQ ID NO |
| | | | | GGG CTC | | | AGG CTG | | |
| | | | | TAT GGA | | | GGT TGA | | |
| | | | | TT | | | TA | | |
| Ampka1 | Mouse | NM_001013367.3 | 5′- | TGT TCC | -3′ | 5′- | ATA ATT | -3′ | SEQ ID NO |
| | | | | AGC | | | GGG | | |
| | | | | AGA | | | TGA | | |
| | | | | TCC TTT | | | GCC | | |
| | | | | CC | | | ACA GC | | |
| Ampka2 | Mouse | XM_006502651.3 | 5′- | GGG | -3′ | 5′- | GTG TTC | -3′ | SEQ ID NO |
| | | | | TGA | | | TCC AAT | | |
| | | | | AGA | | | CTT CAC | | |
| | | | | TCG | | | TTT G | | |
| | | | | GAC | | | | | |
| | | | | ACT | | | | | |
| | | | | ACG T | | | | | |
| Ampkb1 | Mouse | NM_031869.2 | 5′- | CAT CCT | -3′ | 5′- | GAG | -3′ | SEQ ID NO |
| | | | | CCC GCC | | | CAC CAT | | |
| | | | | ACA CCT | | | CAC TCC | | |
| | | | | GC | | | ATC CT | | |
| Ampkb2 | Mouse | NM_182997.2 | 5′- | GGG | -3′ | 5′- | CTG CTG | -3′ | SEQ ID NO |
| | | | | AAA | | | CCA | | |
| | | | | GGA | | | GGG | | |
| | | | | GCA | | | TAC | | |
| | | | | CAA | | | AAA C | | |
| | | | | GAT C | | | | | |
| Idh3b | Mouse | NM_130884.4 | 5′- | ATC TGA | -3′ | 5′- | TAC GTT | -3′ | SEQ ID NO |
| | | | | GCG | | | GGC | | |
| | | | | AGG | | | AAA | | |
| | | | | TGC | | | CAA | | |
| | | | | AGA AT | | | ATC CA | | |
| Fh1 | Mouse | NM_010209.2 | 5′- | AGC | -3′ | 5′- | CGC | -3′ | SEQ ID NO |
| | | | | AAT | | | ATA CTG | | |
| | | | | GCA TAT | | | GAC TTG | | |
| | | | | TGC TGC | | | CTG AA | | |
| | | | | TG | | | | | |
| Mdh1 | Mouse | NM_001316675.1 | 5′- | GAA | -3′ | 5′- | TCG | -3′ | SEQ ID NO |
| | | | | GCC CTG | | | ACA | | |
| | | | | AAA | | | CGA | | |
| | | | | GAC | | | ACT CTC | | |
| | | | | GAC AG | | | CCT CT | | |
| Cpt1a | Mouse | NM_013495.2 | 5′- | GCT | -3′ | 5′- | CAC TGT | -3′ | SEQ ID NO |
| | | | | GGG | | | AGC | | |
| | | | | CTA CTC | | | CTG GTG | | |
| | | | | AGA | | | GGT TT | | |
| | | | | GGA TG | | | | | |
| Pdhb | Mouse | NM_024221.3 | 5′- | TCG | -3′ | 5′- | AGG | -3′ | SEQ ID NO |
| | | | | AAG | | | CAT | | |
| | | | | CCA | | | AGG | | |
| | | | | TAG | | | GAC | | |
| | | | | AAG | | | ATC | | |
| | | | | CCA GT | | | AGC AC | | |
| Acss1 | Mouse | NM_080575.2 | 5′- | ACC | -3′ | 5′- | TCC TCC | -3′ | SEQ ID NO |
| | | | | AGA | | | AGG | | |
| | | | | TCC TGG | | | GTA | | |
| | | | | TGG | | | GTG | | |
| | | | | TGA AG | | | GTG TC | | |
| Acss2 | Mouse | NM_019811.3 | 5′- | GCT TCT | -3′ | 5′- | CCC | -3′ | SEQ ID NO |
| | | | | TTC CCA | | | GGA | | |
| | | | | TTC TTC | | | CTC ATT | | |
| | | | | GGT | | | CAG | | |
| | | | | | | | GAT TG | | |
| Acly | Mouse | NM_001199296.1 | 5′- | GAT | -3′ | 5′- | GGT | -3′ | SEQ ID NO |
| | | | | GAA | | | ATG TCG | | |
| | | | | GTG | | | GCT | | |
| | | | | GCA CCT | | | GAA | | |
| | | | | GCA | | | GAG | | |
| | | | | AAG | | | GGT | | |
| Sod1 | Mouse | NM_011434.1 | 5′- | CCA | -3′ | 5′- | TTG TTT | -3′ | SEQ ID NO |
| | | | | GTG | | | CTC ATG | | |
| | | | | CAG | | | GAC | | |
| | | | | GAC CTC | | | CAC CA | | |
| | | | | ATT TT | | | | | |
| Sod2 | Mouse | NM_013671.3 | 5′- | CCG | -3′ | 5′- | GCT TGA | -3′ | SEQ ID NO |
| | | | | AGG | | | TAG CCT | | |
| | | | | AGA | | | CCA | | |
| | | | | AGT | | | GCA AC | | |
| | | | | ACC | | | | | |
| | | | | ACG AG | | | | | |
| Il1b | Mouse | NM_008361.4 | 5′- | GCA | -3′ | 5′- | ATC TTT | -3′ | SEQ ID NO |
| | | | | ACT GTT | | | TGG | | |
| | | | | CCT | | | GGT | | |
| | | | | GAA | | | CGC TCA | | |
| | | | | CTC AAC | | | ACT | | |
| | | | | T | | | | | |
| Atf4 | Mouse | NM_009716.3 | 5′- | CCT TCG | -3′ | 5′- | CTG TCC | -3′ | SEQ ID NO |
| | | | | ACC | | | CGG | | |
| | | | | AGT | | | AAA | | |
| | | | | CGG GTT | | | AGG | | |
| | | | | TG | | | CAT CC | | |
| mtDNA | Mouse | NC_005089.1 | 5′- | CCC | -3′ | 5′- | GAT | -3′ | SEQ ID NO |
| | | | | AGC | | | GGT TTG | | |
| | | | | TAC TAC | | | GGA | | |
| | | | | CAT CAT | | | GAT | | |
| | | | | TCA AGT | | | TGG TTG | | |
| | | | | | | | ATG | | |
| 5S rRNA | Mouse | NR_030686.1 | 5′- | GGC | -3′ | 5′- | CAG | -3′ | SEQ ID NO |
| | | | | CAT ACC | | | CAC | | |
| | | | | ACC CTG | | | CCG | | |
| | | | | AAC GC | | | GTA TTC | | |
| | | | | | | | CCA GG | | |
| 12S rRNA | Mouse | NC_005089.1 | 5′- | CAA | -3′ | 5′- | GAG | -3′ | SEQ ID NO |
| | | | | ACT | | | GGT | | |
| | | | | GGG | | | GAC | | |
| | | | | ATT | | | GGG | | |
| | | | | AGA | | | CGG TGT | | |
| | | | | TAC CCC | | | GT | | |
| | | | | ACT AT | | | | | |
| miRNA | | | | | | | | | |
| miR-802 | Mouse | MIMAT0004188 | | UCA | | | | | SEQ ID NO |
| | | | | GUA | | | | | |
| | | | | ACA | | | | | |
| | | | | AAG | | | | | |
| | | | | AUU | | | | | |
| | | | | CAU | | | | | |
| | | | | CCU U | | | | | |
| miR-107 | Mouse | MIMAT0000647 | | AGC | | | | | SEQ ID NO |
| | | | | AGC | | | | | |
| | | | | AUU | | | | | |
| | | | | GUA | | | | | |
| | | | | CAG | | | | | |
| | | | | GGC | | | | | |
| | | | | UAU CA | | | | | |
| miR-320 | Mouse | MIMAT0000666 | | AAA | | | | | SEQ ID NO |
| | | | | AGC | | | | | |
| | | | | UGG | | | | | |
| | | | | GUU | | | | | |
| | | | | GAG | | | | | |
| | | | | AGG | | | | | |
| | | | | GCG A | | | | | |
| miR-29b | Mouse | MIMAT0000127 | | UAG | | | | | SEQ ID NO |
| | | | | CAC | | | | | |
| | | | | CAU | | | | | |
| | | | | UUG | | | | | |
| | | | | AAA | | | | | |
| | | | | UCA | | | | | |
| | | | | GUG UU | | | | | |
| miR-33 | Mouse | MIMAT0004666 | | CAA | | | | | SEQ ID NO |
| | | | | UGU | | | | | |
| | | | | UUC | | | | | |
| | | | | CAC | | | | | |
| | | | | AGU | | | | | |
| | | | | GCA | | | | | |
| | | | | UCA C | | | | | |
| miR-93 | Mouse | MIMAT0004636 | | ACU | | | | | SEQ ID NO |
| | | | | GCU | | | | | |
| | | | | GAG | | | | | |
| | | | | CUA | | | | | |
| | | | | GCA | | | | | |
| | | | | CUU | | | | | |
| | | | | CCC G | | | | | |
| miR-103 | Mouse | MIMAT0000546 | | AGC | | | | | SEQ ID NO |
| | | | | AGC | | | | | |
| | | | | AUU | | | | | |
| | | | | GUA | | | | | |
| | | | | CAG | | | | | |
| | | | | GGC | | | | | |
| | | | | UAU GA | | | | | |
| miR-130a | Mouse | MIMAT0000141 | | CAG | | | | | SEQ ID NO |
| | | | | UGC | | | | | |
| | | | | AAU | | | | | |
| | | | | GUU | | | | | |
| | | | | AAA | | | | | |
| | | | | AGG | | | | | |
| | | | | GCA U | | | | | |
| miR-27b | Mouse | MIMAT0000126 | | UUC | | | | | SEQ ID NO |
| | | | | ACA | | | | | |
| | | | | GUG | | | | | |
| | | | | GCU | | | | | |
| | | | | AAG | | | | | |
| | | | | UUC | | | | | |
| | | | | UGC | | | | | |
| miR-152 | Mouse | MIMAT0000162 | | UCA | | | | | SEQ ID NO |
| | | | | GUG | | | | | |
| | | | | CAU | | | | | |
| | | | | GAC | | | | | |
| | | | | AGA | | | | | |
| | | | | ACU | | | | | |
| | | | | UGG | | | | | |
| miR-148a | Mouse | MIMAT0000516 | | UCA | | | | | SEQ ID NO |
| | | | | GUG | | | | | |
| | | | | CAC | | | | | |
| | | | | UAC | | | | | |
| | | | | AGA | | | | | |
| | | | | ACU | | | | | |
| | | | | UUG U | | | | | |
| miR-148b | Mouse | MIMAT0000580 | | UCA | | | | | SEQ ID NO |
| | | | | GUG | | | | | |
| | | | | CAU | | | | | |
| | | | | CAC | | | | | |
| | | | | AGA | | | | | |
| | | | | ACU | | | | | |
| | | | | UUG U | | | | | |
| snoRNA202 | Mouse | AF357327 | | GCT GTA | | | | | SEQ ID NO |
| | | | | CTG ACT | | | | | |
| | | | | TGA | | | | | |
| | | | | TGA | | | | | |
| | | | | AAG | | | | | |
| | | | | TAC TTT | | | | | |
| | | | | TGA | | | | | |
| | | | | ACC CTT | | | | | |
| | | | | TTC CAT | | | | | |
| | | | | CTG ATG | | | | | |
| pri-miR | | | | | | | | | SEQ ID NO |
| pri-mir- | Mouse | MI0004249 | | UCA | | | | | |
| 802 | | | | GUA | | | | | SEQ ID NO |
| | | | | ACA | | | | | |
| | | | | AAG | | | | | |
| | | | | AUU | | | | | |
| | | | | CAU | | | | | |
| | | | | CCU U | | | | | |
| pri-mir- | Mouse | MI0000684 | | AGC | | | | | SEQ ID NO |
| 107 | | | | AGC | | | | | |
| | | | | AUU | | | | | |
| | | | | GUA | | | | | |
| | | | | CAG | | | | | |
| | | | | GGC | | | | | |
| | | | | UAU CA | | | | | |
| pri-mir- | Mouse | MI0000587 | | AGC | | | | | SEQ ID NO |
| 103-1 | | | | AGC | | | | | |
| | | | | AUU | | | | | |
| | | | | GUA | | | | | |
| | | | | CAG | | | | | |
| | | | | GGC | | | | | |
| | | | | UAU GA | | | | | |
| pri-mir- | Mouse | MI0000588 | | AGC | | | | | SEQ ID NO |
| 103-2 | | | | AGC | | | | | |
| | | | | AUU | | | | | |
| | | | | GUA | | | | | |
| | | | | CAG | | | | | |
| | | | | GGC | | | | | |
| | | | | UAU GA | | | | | |
| pri-mir- | Mouse | MI0000156 | | GCU | | | | | SEQ ID NO |
| 130a | | | | CUU | | | | | |
| | | | | UUC | | | | | |
| | | | | ACA | | | | | |
| | | | | UUG | | | | | |
| | | | | UGC | | | | | |
| | | | | UAC U | | | | | |
| pri-miR- | Mouse | MI0000174 | | UAG | | | | | SEQ ID NO |
| 152 | | | | GUU | | | | | |
| | | | | CUG | | | | | |
| | | | | UGA | | | | | |
| | | | | UAC | | | | | |
| | | | | ACU | | | | | |
| | | | | CCG | | | | | |
| | | | | ACU | | | | | |
| pri-miR- | Mouse | MI0000550 | | UCA | | | | | SEQ ID NO |
| 148a | | | | GUG | | | | | |
| | | | | CAC | | | | | |
| | | | | UAC | | | | | |
| | | | | AGA | | | | | |
| | | | | ACU | | | | | |
| | | | | UUG U | | | | | |
| pri-miR- | Mouse | MI0000617 | | GAA | | | | | SEQ ID NO |
| 148b | | | | GUU | | | | | |
| | | | | CUG | | | | | |
| | | | | UUA | | | | | |
| | | | | UAC | | | | | |
| | | | | ACU | | | | | |
| | | | | CAG | | | | | |
| | | | | GCU | | | | | |

High-throughput sequencing of miRNA. Liver tissues were excised from mice, and stored in −80° C. after RNAlater (Invitrogen) treatment. Liver tissue was homogenized with QIAzol (Qiagen). Total RNA, including miRNA, was extracted using the miRNeasy Micro Kit (Qiagen). High-throughput sequencing of miRNA was processed according to TruSeq Small RNA Sample Preparation Guide (Illumina). Briefly, the total RNA was run on an agarose gel and the band corresponding to the size of miRNAs was cut out for further processing. Sequencing adapters were ligated to the size-selected RNA molecules, followed by reverse transcription to obtain the cDNA library, which was subsequently sequenced by Illumina HiSeq2500.

Bioinformatic analysis of miRNA seq. Adapter sequences were removed with fastx_toolkit (v0.0.14, -a TGGAATTCTCGGGTGCCAAGG-1 (SEQ ID NO: 1) 15 -M 20-c, http://hannonlab.csh1.edu/fastx_toolkit/) for NCD (FIG. 1A) and S961 (FIG. 3D) samples, and TrimGalore (v0.4.3, -e 0.1 -q 20 -O 1 -a AGATCGGAAGAGCACACGTC, (SEQ ID NO: 2) https://www.bioinformatics.babraham.ac.uk/projects/trimgalore/), and Cutadapt (v1.9.1, http://cutadapt.readthedocs.io) for HFD samples (FIG. 5A), with reads that became shorter than 15 bases, remaining reads were filtered with the FASTX package (v0.0.14), using a quality threshold of 20 over at least 90 percent of the read. To get raw counts for each mature miRNA, miRExpress (v2.1.4, http://mirexpress.mbc.nctu.edu.tw/) was used to align and create expression count profiles with default parameters. The alignment step (alignmentSlMD) uses a miRNA precursor file (mmu_precursor.txt), supplied with the miRExpress v2.1.4 and the analysis step uses mmu_miRNA.txt with mature miRNA information for precursor sequences. Raw counts were normalized using DESeq2 (v1.18.0, http://bioconductor.org/packages/release/bioc/html/DESeq2.html), to compare mature miRNAs' relative abundance. Differentially expressed miRNAs were predicted also using DESeq2. For NCD (FIG. 1A) and HFD samples (FIG. 5A), Applicant applied |fold change|>2× and adjusted P (FDR BH)<0.05 as filters. For S961 (FIG. 3D), Applicant applied |fold change|>1.25× and p<0.0005. Heatmaps were created by the pheatmap package (https://cran.r-project.org/web/packages/pheatmap/index.html).

miRNA target pathway enrichment analysis. To predict the enriched target pathways, Applicant used the mirPath web-server (v3, http://snf-515788.vm.okeanos.grnet.gr), based on DIANA-microT-CDS algorithm. Applicant chose KEGG (http://www.genome.jp/kegg) database as a reference and p<0.05 and MicroT threshold<0.8 as filters to get significantly enriched KEGG pathways.

Protein extraction and immunoblot analysis. To prepare protein lysates, cells were washed with cold PBS, followed by lysis in cold mammalian cell lysis buffer [MCLB: 50 mM Tris-HCl (pH 7.4), 150 mM NaCl, 10 mM NaF, 5% glycerol, 1% NP-40, 1% protease and phosphatase inhibitor cocktail]. After homogenization on ice, the cell lysates were centrifuged, and the supernatants were used for western blot analyses. For preparation of liver tissue lysates, the tissues were placed in a cold MCLB and homogenized on ice. The tissue lysates were centrifuged, and the supernatants were used for further experiments.

Morphological and immunohistochemical analysis of hepatic and pancreatic tissues. Liver and pancreas were taken, fixed with 10% formalin, and paraffin-embedded sections were prepared for further analysis. Paraffin sections were stained with H&E and Periodic Acid-Schiff (PAS) for morphology analyses. For the immunohistochemical staining, the following primary antibodies were used: guinea pig polyclonal anti-insulin (Abcam) and rabbit monoclonal anti-glucagon (Abcam). The following secondary antibodies were used: Alexa Fluor 488-conjugated AffiniPure Goat Anti-Guinea Pig (Jackson Immunoresearch) and Alexa Fluor 594-conjugated AffiniPure Goat Anti-rabbit IgG (Jackson Immunoresearch). The nuclei were stained using 4′,6-diamidino-2-phenylindole (DAPI), and sections were preserved using fluorescence mounting medium (Electron Microscopy Science). Images were acquired on a Nikon 90i Upright. ImageJ was used to process the images.

Palmitate and acetate oxidation assays. Seahorse Bioscience XFe96 extracellular Flux Analyzers were used⁵⁷ to detect palmitate oxidation in primary hepatocytes. Palmitate oxidation was measured by oxygen consumption rate (OCR) with modification. Primary hepatocytes were seeded at a density of 6,000 cells per well of a XFe96 cell culture microplate and incubated in William E supplemented with 10% FBS. Next day, cells were cultured with DMEM (5.5 mM glucose) supplemented with 10% FBS and 2 mM Glutamax for 16 hours. Then, these cells were incubated in DMEM (5.5 mM glucose) supplemented with 1% FBS, 1 mM Glutamax, and 0.5 mM carnitine for 2 hours and then equilibrated for 1 hour in palmitate oxidation assay medium (111 mM NaCl, 4.7 mM KCl, 1.25 mM CaCl₂, 2 mM MgSO₄, 1.2 mM NaH₂PO₄) supplemented with 2.5 mM glucose, 0.5 mM carnitine, and 5 mM HEPES at 37° C. for 1 hour. 15 minutes prior to the assay, additional 400 μM of Etomoxir or vehicle was added. Palmitate-BSA or BSA was added to the microplate just prior to starting the assay. Sequential injections of 2 μM oligomycin, 2 μM phenylhydrazone, and 1 μM rotenone/1 μM antimycin A1 were used to examine mitochondrial oxidative status. For acetate oxidation assay, sodium acetate (1M, pH 7.4) was prepared followed by filtering. Sequential injection of 5 mM acetate, 2 μM oligomycin, 2 μM phenylhydrazone and 1 μM rotenone/1 μM antimycin A1 were used to examine mitochondrial oxidative status. Each readout was normalized to total cellular protein levels.

Pyruvate oxidation assay and glycolysis stress test. Primary hepatocytes were isolated and seeded at a density of 6,000 cells per well of a XFe96 cell culture microplate and incubated in William E supplemented with 10% FBS. Next day, cells were cultured with DMEM (5.5 mM glucose) supplemented with 10% FBS. Next day, prior to performing an assay, growth medium in the wells of XF cell plate was exchanged with 175 μl of XF base medium (pH 7.4) containing no exogenous fuel substrate supplementation (Seahorse Bioscience) at 37° C. for 1 hour. Sequential injection of 2 mM pyruvate, 2 μM oligomycin, 2 μM phenylhydrazone, and 1 μM rotenone/1 μM antimycin A1 were used to examine mitochondrial oxidative status. For glycolysis stress test, sequential injection of 10 mM glucose, 2 μM oligomycin, and 2-DG (2-deoxy-glycose, a glucose analog) were used to examine glycolysis stress. Each readout was normalized to total proteins.

ATP/ADP ratio assay. The ATP/ADP ratio in the mouse liver extract or primary hepatocytes was determined using a bioluminescent ATP/ADP Ratio Assay Kit (Abcam) according to the manufacturer's instructions. For mouse liver samples, the samples were immediately frozen in liquid nitrogen and powdered with a mortar. Tissue powders were suspended in the provided lysis buffer (10 μl/mg of tissue powder) for 5 min at room temperature, followed by centrifugation at 10,000 G for 1 min to pellet insoluble material. For mouse primary hepatocytes, cells were plated at a density of 0.8×10⁵ cells per well of 24-well plate for overnight. Next day, cells were cultured with XF base media or DMEM no glucose and glutamine media (Gibco: A1443001) in the presence or absence of 5 mM Sodium Palmitate without FBS for 2 hours. Data were normalized by the amount of protein present in the supernatant.

ATP and ADP assays. The ATP or ADP in the liver extract was determined using a ATP Assay Kit (Abcam) or ADP Assay Kit (Abcam), respectively, according to the manufacturer's instructions. Data were normalized by the amount of protein present in the supernatant.

Protein synthesis analysis. Click-iT labeling technology was used for the detection of nascent protein synthesis in cells according to manufacturer's instructions (Thermo Fisher Scientific). Mouse primary hepatocytes were seeded at 0.6×10⁶ cells/well in a 6-well plate. Cells were then incubated in methionine- and cysteine-free DMEM containing 25 μM of azide-linked methionine analog AHA in the presence or absence of 200 μM phenformin or 10 μM Rotenone for 5 hours. Azide-labeled protein lysate from harvested cells was determined by using Click-iT® TAMRA Protein Analysis Kit according to manufacturer's instructions (Thermo Fisher Scientific) and Typhoon FLA9500 scanner (GE Healthcare) with the excitation at 532 nm. Coomassie Brilliant Blue (CBB)-based staining (Thermo Fisher Scientific; GelCode Blue) for total protein served as a loading control.

Mitochondrial DNA copy number. Total DNA were purified from mouse liver using GeneJet Genomic DNA purification kit according to the manufacturer's instruction (Thermo Fisher Scientific). Mitochondrial DNA copy number was detected by qPCR⁶¹.

Glucose production assay. Mouse primary hepatocytes were cultured in 12-well plates (0.4×10⁶ cells per well) in William E supplemented with 10% FBS. Next day, cells were cultured with 1 ml of DMEM (5.5 mM glucose) supplemented with 10% FBS. Post plating for 21 hours, cells were washed twice with PBS and were subjected 3-4 hours to serum starvation with FBS-free DMEM (5.5 mM glucose). After washing twice with PBS, cells were cultured in 0.4 ml of glucose production buffer consisting of glucose-free DMEM (pH 7.4) without phenol red supplemented with 20 mM sodium lactate, 2 mM sodium pyruvate, 2 mM L-glutamine and 15 mM HEPES⁶². Cells were incubated at 37° C. for 4.5 hours with or without Bt-cAMP or pCPT-cAMP. Both medium and cells were collected. The glucose concentration was measured with the Autokit Glucose (WAKO) and was normalized by the total protein content.

Mitochondrial isolation. Liver tissue samples were minced and kept in ice-cold PBS containing proteinase inhibitor immediately after harvest. Tissues were then homogenized in resuspension buffer (RSB)/EDTA (10 mM Tris pH 6.7, 10 mM NaCl, 0.1 mM EDTA pH 8.0) containing proteinase inhibitor. The homogenized samples were filtered through 30-μm filter and sucrose concentration was then adjusted to 250 mM by adding 2 M sucrose. The suspension was centrifuged at 2,400 rpm for 3 min at 4° C. and the supernatant was collected for further separation. Crude mitochondrial for functional analysis were sedimented from the supernatant by centrifugation at 9650 G for 10 min at 4° C. To prepare pure mitochondria, the crude mitochondria were resuspended in ice-cold separation buffer, mixed with anti-TOM22 MicroBeads and enriched on a MACS column (Miltenyi Biotec). Magnetically purified mitochondria were incubated with 100 μg/ml of RNase for 30 min on ice and 10× volume of T10E20/sucrose was used to wash the mitochondria. Isolated mitochondria were pelleted and kept in −80° C. until use.

Luciferase assay. Luciferase plasmids harboring the Ampka1 3′ UTR were generated as follows. Ampka1 3′ UTR were amplified by using primer: 5′-CCCAGAATTCCATTTAAGTTACAGCCTG-3′ (SEQ ID NO 3) and 5′GCATCTCGAGGTTCCTTTCATGAGAAATCAAC-3′ (SEQ ID NO 4), and cloned into EcoRI and XhoI restriction enzyme sites of pEZX-MT06 (GeneCopoeia). The EcoRI and XhoI sites are shown in italics. PCR was performed using Phusion High-Fidelity DNA polymerase (New England BioLabs). Mutagenesis of the 3′ UTR was performed with the QuickChange Lightining Site-Directed Mutagenesis Kit (Agilent Technologies) according to the manufacturer's instructions. Primer sequences used to mutate the miR-148/miR-152 binding site in the Ampka1 are the following: 5′-CATGATAGCTTGCATAAAAG ATGACGCTATAGTTTAACGTCTGATTTCCGGACAAAA ATG-3′(SEQ ID NO 5) and 5′-CATTTTTGTCCGGAAATCAGACGTTAAACTATAGCGTCATCTTTTATGC AAGCTATC ATG-3′(SEQ ID NO 6). The mutated residues are indicated in bold. Mouse primary hepatocytes were plated at a density of 0.8×10⁵ cells per well of 24-well plate, and transfected with 0.5 μg of each control (pEZX-MT06), Ampka1 (Prkaa1) (MmiT024101-MT06) and Ampka1 mutant using Lipofectamine 3000 (Life Technologies) according to the manufacturer's instructions. The transfected cells were incubated with DMEM (5.5 mM glucose) with 10% FBS media for 8 hours. Luciferase activities were measured with Luc-Pair Duo-Luciferase Assay Kit 2.0 (GeneCopoeia) and GLOMAX 96 Microplate Luminometer (Promega).

Polysome profiling. Preparations of cellular extracts for polysome profiles, sucrose gradient centrifugation, and profile recording have been previously described⁶³. Heparin was omitted from lysates used to analyze Ampka1, Cs, and β-actin mRNAs by qRT-PCR due to its inhibitory effect on the PCR. Sucrose gradient fractions were collected by upward displacement, and 50 pg of synthetic luciferase mRNA (Promega) and 15 μg of GlycoBlue (Life Technologies) were added to each fraction to control for extraction and PCR efficiency and to improve RNA recovery, respectively. Extraction and precipitation of RNA from sucrose fractions have been previously described⁶³. Precipitated RNA was washed twice with ice-cold 70% ethanol, dried, and resuspended in RNase-free H2O. Equal volumes of RNA from each fraction were subjected to cDNA synthesis and qRT-PCR analysis. Levels of Ampka1, Cs, and β-actin mRNAs in each fraction were normalized to luciferase mRNA and plotted as the percentage of total mRNAs from all 12 fractions.

Statistical analysis. Experimental results were shown as the mean±SEM. The mean values for biochemical data from each group were compared by Student's t-test. Comparisons between multiple time points were analyzed using repeated-measures analysis of variance, two-way ANOVA. In all tests, p<0.05 was considered significant.

Data availability. The RNA-Seq data generated in this study have been deposited in the NCBI Sequence Read Archive (SRA, http://www.ncbi.nlm nih.gov/sra) under the BioProject ID PRJNA395686. The accession codes are SAMN07415658, SAMN07415659, SAMN07415660, SAMN07415661, SAMN07415662, and SAMN07415663 for the NCD study, SAMN07415764, SAMN07415766, SAMN07415768, SAMN07416029, SAMN07415767, SAMN07415769, SAMN07415765, SAMN0741630, SAMN07416036, SAMN07416037, SAMN07416038, and SAMN07416039 for the 5961 study, and SAMN07413900, SAMN07413901, SAMN07413906, SAMN07413907, SAMN07413949, and SAMN07413951 for the HFD study.

### REFERENCES

1. Smith, B. W. & Adams, L. A. Nonalcoholic fatty liver disease and diabetes mellitus:pathogenesis and treatment. Nature reviews. Endocrinology 7, 456-465, doi:10.1038/nrendo.2011.72 (2011).

2. Perry, R. J., Samuel, V. T., Petersen, K. F. & Shulman, G. I. The role of hepatic lipids in hepatic insulin resistance and type 2 diabetes. Nature 510, 84-91, doi:10.1038/nature13478 (2014).

3. Durnin, J. V. G. A. Basal metabolic rate in man (FAO/WHO/UNU, 1981).

4. Buttgereit, F. & Brand, M. D. A hierarchy of ATP-consuming processes in mammalian cells. The Biochemical journal 312 (Pt 1), 163-167 (1995).

5. Brown, G. C. Control of respiration and ATP synthesis in mammalian mitochondria and cells. The Biochemical journal 284 (Pt 1), 1-13 (1992).

6. Khamzina, L., Veilleux, A., Bergeron, S. & Marette, A. Increased activation of the mammalian target of rapamycin pathway in liver and skeletal muscle of obese rats: possible involvement in obesity-linked insulin resistance. Endocrinology 146, 1473-1481, doi:10.1210/en.2004-0921 (2005).

7. Fu, S. et al. Polysome profiling in liver identifies dynamic regulation of endoplasmic reticulum translatome by obesity and fasting. PLoS genetics 8, e1002902, doi:10.1371/journal.pgen.1002902 (2012).

8. Oyadomari, S., Harding, H. P., Zhang, Y., Oyadomari, M. & Ron, D. Dephosphorylation of translation initiation factor 2alpha enhances glucose tolerance and attenuates hepatosteatosis in mice. Cell metabolism 7, 520-532, doi:10.1016/j.cmet.2008.04.011 (2008).

9. Yecies, J. L. et al. Akt stimulates hepatic SREBP1c and lipogenesis through parallel mTORC1-dependent and independent pathways. Cell metabolism 14, 21-32, doi:10.1016/j.cmet.2011.06.002 (2011).

10. Arner, P. & Kulyte, A. MicroRNA regulatory networks in human adipose tissue and obesity. Nature reviews. Endocrinology 11, 276-288, doi:10.1038/nrendo.2015.25 (2015).

11. Ross, S. A. & Davis, C. D. The emerging role of microRNAs and nutrition in modulating health and disease. Annu Rev Nutr 34, 305-336, doi:10.1146/annurev-nutr-071813-105729 (2014).

12. Trajkovski, M. et al. MicroRNAs 103 and 107 regulate insulin sensitivity. Nature 474, 649-653, doi:10.1038/nature10112 (2011).

13. Kornfeld, J. W. et al. Obesity-induced overexpression of miR-802 impairs glucose metabolism through silencing of Hnflb. Nature 494, 111-115, doi:nature11793 [pii] 10.1038/nature11793 (2013).

14. Wagschal, A. et al. Genome-wide identification of microRNAs regulating cholesterol and triglyceride homeostasis. Nature medicine 21, 1290-1297, doi:10.1038/nm.3980 (2015).

15. Goedeke, L. et al. MicroRNA-148a regulates LDL receptor and ABCA1 expression to control circulating lipoprotein levels. Nature medicine 21, 1280-1289, doi:10.1038/nm.3949 (2015).

16. Soronen, J. et al. Novel hepatic microRNAs upregulated in human nonalcoholic fatty liver disease. Physiol Rep 4, doi:10.14814/phy2.12661 (2016).

17. Ipsaro, J. J. & Joshua-Tor, L. From guide to target: molecular insights into eukaryotic RNA-interference machinery. Nature structural & molecular biology 22, 20-28, doi:10.1038/nsmb.2931 (2015).

18. Meister, G. Argonaute proteins: functional insights and emerging roles. Nature reviews. Genetics 14, 447-459, doi:10.1038/nrg3462 (2013).

19. Winter, J., Jung, S., Keller, S., Gregory, R. I. & Diederichs, S. Many roads to maturity:microRNA biogenesis pathways and their regulation. Nature cell biology 11, 228-234, doi:10.1038/ncb0309-228 (2009).

20. Bartel, D. P. MicroRNAs: target recognition and regulatory functions. Cell 136, 215-233, doi:10.1016/j.ce11.2009.01.002 (2009).

21. Cheloufi, S., Dos Santos, C. 0., Chong, M. M. & Hannon, G. J. A dicer-independent miRNA biogenesis pathway that requires Ago catalysis. Nature 465, 584-589, doi:10.1038/nature09092 (2010).

22. Yang, J. S. et al. Conserved vertebrate mir-451 provides a platform for Dicer-independent, Ago2-mediated microRNA biogenesis. Proceedings of the National Academy of Sciences of the United States of America 107, 15163-15168, doi:10.1073/pnas.1006432107 (2010).

23. O'Carroll, D. et al. A Slicer-independent role for Argonaute 2 in hematopoiesis and the microRNA pathway. Genes & development 21, 1999-2004, doi:10.1101/gad.1565607 (2007).

24. Liu, J. et al. Argonaute2 is the catalytic engine of mammalian RNAi. Science 305, 1437-1441, doi:10.1126/science.1102513 (2004).

25. Valdmanis, P. N. et al. Expression determinants of mammalian argonaute proteins in mediating gene silencing. Nucleic acids research 40, 3704-3713, doi:10.1093/nar/gkr1274 (2012).

26. Ha, M. & Kim, V. N. Regulation of microRNA biogenesis. Nature reviews. Molecular cell biology 15, 509-524, doi:10.1038/nrm3838 (2014).

27. Carrer, M. et al. Control of mitochondrial metabolism and systemic energy homeostasis by microRNAs 378 and 378*. Proceedings of the National Academy of Sciences of the United States of America 109, 15330-15335, doi:10.1073/pnas.1207605109 (2012).

28. Lu, L. et al. MicroRNA-130a and -130b enhance activation of hepatic stellate cells by suppressing PPARgamma expression: A rat fibrosis model study. Biochemical and biophysical research communications 465, 387-393, doi:10.1016/j.bbrc.2015.08.012 (2015).

29. Huang, J. Y. et al. MicroRNA-130a can inhibit hepatitis B virus replication via targeting PGClalpha and PPARgamma. Rna 21, 385-400, doi:10.1261/rna.048744.114 (2015).

30. Park, J. E. et al. Dicer recognizes the 5′ end of RNA for efficient and accurate processing. Nature 475, 201-205, doi:10.1038/nature10198 (2011).

31. Tsutsumi, A., Kawamata, T., Izumi, N., Seitz, H. & Tomari, Y. Recognition of the pre-miRNA structure by Drosophila Dicer-1. Nature structural & molecular biology 18, 1153-1158, doi:10.1038/nsmb.2125 (2011).

32. Liu, Y. P. et al. Mechanistic insights on the Dicer-independent AGO2-mediated processing of AgoshRNAs. RNA biology 12, 92-100, doi:10.1080/15476286.2015.1017204 (2015).

33. Schaffer, L. et al. A novel high-affinity peptide antagonist to the insulin receptor. Biochemical and biophysical research communications 376, 380-383, doi:10.1016/j.bbrc.2008.08.151 (2008).

34. Yi, P., Park, J. S. & Melton, D. A. Betatrophin: a hormone that controls pancreatic beta cell proliferation. Cell 153, 747-758, doi:10.1016/j.cell.2013.04.008 (2013).

35. Perry, R. J. et al. Acetate mediates a microbiome-brain-beta-cell axis to promote metabolic syndrome. Nature 534, 213-217, doi:10.1038/nature18309 (2016).

36. Lu, Y. C. et al. ELAVL1 modulates transcriptome-wide miRNA binding in murine macrophages. Cell reports 9, 2330-2343, doi:10.1016/j.celrep.2014.11.030 (2014).

37. Hardie, D. G. AMPK-Sensing Energy while Talking to Other Signaling Pathways. Cell metabolism 20, 939-952, doi:10.1016/j.cmet.2014.09.013 (2014).

38. Zhang, B. B., Zhou, G. & Li, C. AMPK: an emerging drug target for diabetes and the metabolic syndrome. Cell metabolism 9, 407-416, doi:10.1016/j.cmet.2009.03.012 (2009).

39. Toyama, E. Q. et al. Metabolism. AMP-activated protein kinase mediates mitochondrial fission in response to energy stress. Science 351, 275-281, doi:10.1126/science.aab4138 (2016).

40. Foretz, M., Guigas, B., Bertrand, L., Pollak, M. & Viollet, B. Metformin: From Mechanisms of Action to Therapies. Cell metabolism 20, 953-966, doi:10.1016/j.cmet.2014.09.018 (2014).

41. Burkewitz, K., Zhang, Y. & Mair, W. B. AMPK at the nexus of energetics and aging. Cell metabolism 20, 10-25, doi:10.1016/j.cmet.2014.03.002 (2014).

42. Morita, S. et al. One Argonaute family member, Eif2c2 (Ago2), is essential for development and appears not to be involved in DNA methylation. Genomics 89, 687-696, doi:10.1016/j.ygeno.2007.01.004 (2007).

43. Wang, D. et al. Quantitative functions of Argonaute proteins in mammalian development. Genes & development 26, 693-704, doi:10.1101/gad.182758.111 (2012).

44. Lykke-Andersen, K. et al. Maternal Argonaute 2 is essential for early mouse development at the maternal-zygotic transition. Mol Biol Cell 19, 4383-4392, doi:10.1091/mbc.E08-02-0219 (2008).

45. The Jackson Laboratory. 2005. Information obtained from The Jackson Laboratory, Bar Harbor, ME Unpublished : MGI: J:101977

46. Shen, J. et al. EGFR modulates microRNA maturation in response to hypoxia through phosphorylation of AGO2. Nature 497, 383-387, doi:10.1038/nature12080 (2013).

47. Horman, S. R. et al. Akt-mediated phosphorylation of argonaute 2 downregulates cleavage and upregulates translational repression of MicroRNA targets. Molecular cell 50, 356-367, doi:10.1016/j.molce1.2013.03.015 (2013).

48. Cifuentes, D. et al. A novel miRNA processing pathway independent of Dicer requires Argonaute2 catalytic activity. Science 328, 1694-1698, doi:10.1126/science.1190809 (2010).

49. Fu, S. et al. Aberrant lipid metabolism disrupts calcium homeostasis causing liver endoplasmic reticulum stress in obesity. Nature 473, 528-531, doi:10.1038/nature09968 (2011).

50. Crescenzo, R. et al. A possible link between hepatic mitochondrial dysfunction and diet-induced insulin resistance. Eur J Nutr 55, 1-6, doi:10.1007/s00394-015-1073-0 (2016).

51. Singh, R. et al. Autophagy regulates lipid metabolism. Nature 458, 1131-1135, doi:10.1038/nature07976 (2009).

52. Yang, L., Li, P., Fu, S., Calay, E. S. & Hotamisligil, G. S. Defective hepatic autophagy in obesity promotes ER stress and causes insulin resistance. Cell metabolism 11, 467-478, doi:S1550-4131(10)00116-6 [pii] 10.1016/j.cmet.2010.04.005 (2010).

53. Madiraju, A. K. et al. Metformin suppresses gluconeogenesis by inhibiting mitochondrial glycerophosphate dehydrogenase. Nature 510, 542-546, doi:10.1038/nature13270 (2014).

54. Jonas, S. & Izaurralde, E. Towards a molecular understanding of microRNA-mediated gene silencing. Nature reviews. Genetics 16, 421-433, doi:10.1038/nrg3965 (2015).

55. Howell, J. J. et al. Metformin Inhibits Hepatic mTORC1 Signaling via Dose-DependentMechanisms Involving AMPK and the TSC Complex. Cell metabolism 25, 463-471, doi:10.1016/j.cmet.2016.12.009 (2017).

56. Blandino, G. et al. Metformin elicits anticancer effects through the sequential modulation of DICER and c-MYC. Nature communications 3, 865, doi:10.1038/ncomms1859 (2012).

57. Giles, D. A. et al. Thermoneutral housing exacerbates nonalcoholic fatty liver disease in mice and allows for sex-independent disease modeling. Nature medicine 23, 829-838, doi:10.1038/nm.4346 (2017).

58. Stemmer, K. et al. Thermoneutral housing is a critical factor for immune function and diet-induced obesity in C57BL/6 nude mice. Int J Obes (Lond) 39, 791-797, doi:10.1038/ijo.2014.187 (2015).

59. Arruda, A. P. et al. Chronic enrichment of hepatic endoplasmic reticulum-mitochondria contact leads to mitochondrial dysfunction in obesity. Nature medicine 20, 1427-1435, doi:10.1038/nm.3735 (2014).

60. Zhang, C., Seo, J. & Nakamura, T. Cellular Approaches in Investigating Argonaute2-Dependent RNA Silencing. Methods in molecular biology 1680, 205-215, doi:10.1007/978-1-4939-7339-2_14 (2018).

61. Rooney, J. P. et al. PCR based determination of mitochondrial DNA copy number in multiple species. Methods in molecular biology 1241, 23-38, doi:10.1007/978-1-4939-1875-1_3 (2015).

62. Sakai, M. et al. CITED2 links hormonal signaling to PGC-lalpha acetylation in the regulation of gluconeogenesis. Nature medicine 18, 612-617, doi:10.1038/nm.2691 (2012).

63. Teng, T., Mercer, C. A., Hexley, P., Thomas, G. & Fumagalli, S. Loss of tumor suppressor RPL5/RPL11 does not induce cell cycle arrest but impedes proliferation due to reduced ribosome content and translation capacity. Molecular and cellular biology 33, 4660-4671, doi:10.1128/MCB.01174-13 (2013).

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of treating a metabolic disorder in an individual in need thereof, comprising the step of administering a therapeutically effective amount of an agent that interferes with Ago2 activity and/or function in combination with a pharmaceutically acceptable excipient.

2. The method of claim 1, wherein said metabolic disorder is selected from obesity, type II diabetes, heart disease, liver disease, or combinations thereof.

3. The method of claim 1, wherein said metabolic disorder is fatty liver disease.

4. The method of claim 1, wherein said agent that interferes with Ago2 activity and/or function is selected from an inhibitory antibody specific for Ago2, an inhibitory nucleotide specific for Ago2, or a combination thereof.

5. The method of claim 1, wherein said agent that interferes with Ago2 activity and/or function is selected from trypaflavine (TPF) 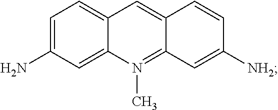
propanoic acid 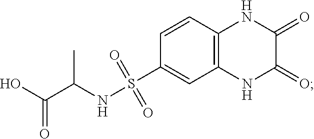 [SMILES: CC(NS(=O)(=O)c1ccc2[nH]c(=O)c(=O)[nH]c2c1)C(=O)O]
aurintricarboxylic acid (ACF) 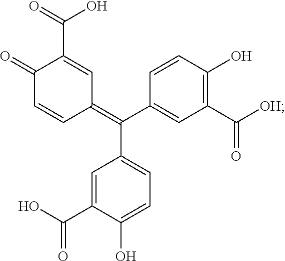 [SMILES: O=C(O)C1=CC(=C(c2ccc(O)c(C(=O)O)c2)c2ccc(O)c(C(=O)O)c2)C=CC1=O]
suramin 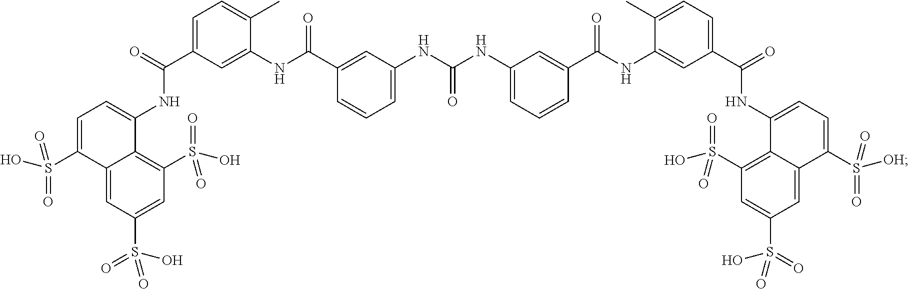 [SMILES: Cc1ccc(C(=O)Nc2ccc(S(=O)(=O)O)c3cc(S(=O)(=O)O)cc(S(=O)(=O)O)c23)cc1NC(=O)c1cccc(NC(=O)Nc2cccc(C(=O)Nc3cc(C(=O)Nc4ccc(S(=O)(=O)O)c5cc(S(=O)(=O)O)cc(S(=O)(=O)O)c45)ccc3C)c2)c1]
oxidopamine HCL 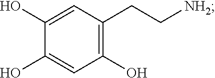 [SMILES: NCCc1cc(O)c(O)cc1O] 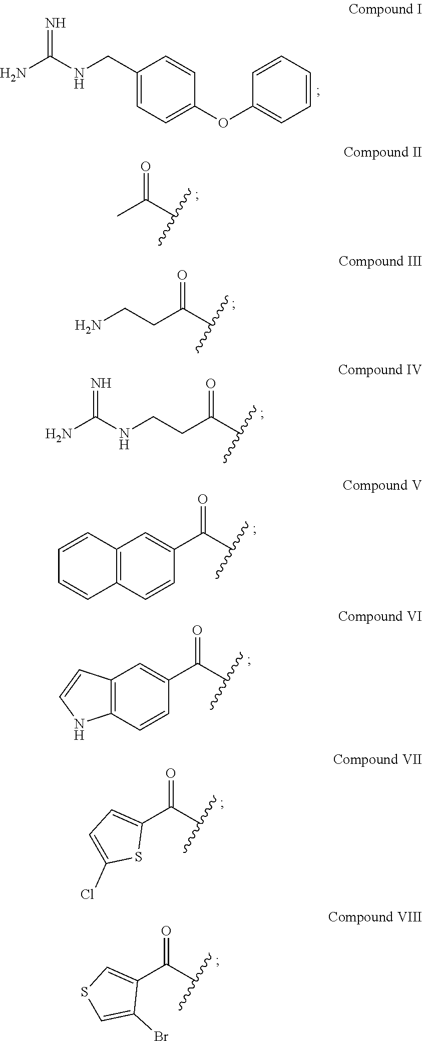
and pharmaceutically acceptable salts thereof and combinations thereof.

6. The method of claim 1, wherein administration of said agent is administered via a route selected from intravenously, orally, topically, parenterally, by inhalation or spray, sublingually in dosage unit formulations, or a combination thereof.

7. A method of improving systemic glucose metabolism in the liver on an individual in need thereof, comprising the step of administering a therapeutically effective amount of an agent that interferes with Ago2 activity and/or function in combination with a pharmaceutically acceptable excipient.

8. The method of claim 7, wherein said agent that interferes with Ago2 activity and/or function is selected from function is selected from trypaflavine (TPF) 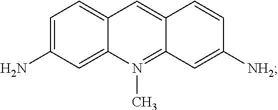 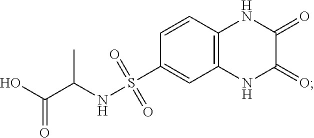 [SMILES: CC(NS(=O)(=O)c1ccc2[nH]c(=O)c(=O)[nH]c2c1)C(=O)O]
aurintricarboxylic acid (ACF) 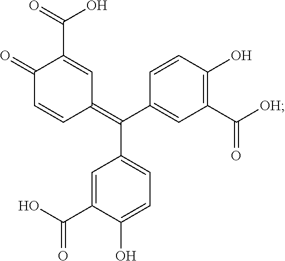 [SMILES: O=C(O)C1=CC(=C(c2ccc(O)c(C(=O)O)c2)c2ccc(O)c(C(=O)O)c2)C=CC1=O]
suramin 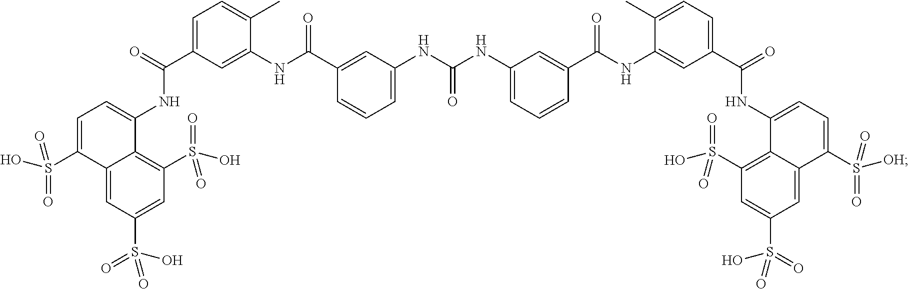 [SMILES: Cc1ccc(C(=O)Nc2ccc(S(=O)(=O)O)c3cc(S(=O)(=O)O)cc(S(=O)(=O)O)c23)cc1NC(=O)c1cccc(NC(=O)Nc2cccc(C(=O)Nc3cc(C(=O)Nc4ccc(S(=O)(=O)O)c5cc(S(=O)(=O)O)cc(S(=O)(=O)O)c45)ccc3C)c2)c1]
oxidopamine HCL 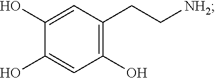 [SMILES: NCCc1cc(O)c(O)cc1O] 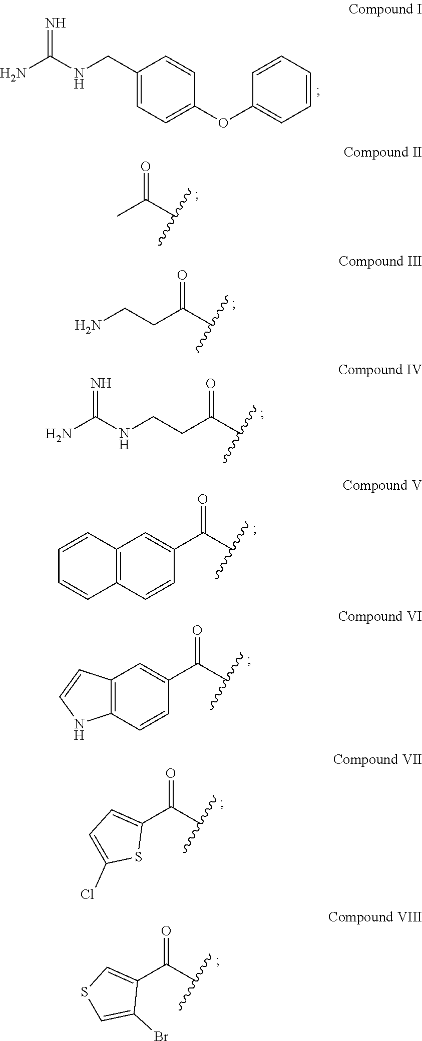
and pharmaceutically acceptable salts thereof and combinations thereof.

9. A therapeutic kit comprising a) the composition according to claim 1 and b) a means for delivery of the composition to a human.

10. An article of manufacture comprising a) a container comprising a label; and b) a composition according to claim 1, wherein the label indicates that the composition is to be administered to an individual in need of treatment for a systemic glucose metabolism related condition.


SEQUENCE LISTING (357 sequences)

SEQ ID NO: 1 - RNA, 24 nt, Artificial Sequence
  miR-802-5p
aucaguaacaaagauucauccuug

SEQ ID NO: 2 - RNA, 25 nt, Artificial Sequence
  miR-802-5p
aucaguaacaaagauucauccuugu

SEQ ID NO: 3 - RNA, 25 nt, Artificial Sequence
  miR-802-5p
guucaguaacaaagauucauccuug

SEQ ID NO: 4 - RNA, 23 nt, Artificial Sequence
  miR-802-5p
aucaguaacaaagauucauccuu

SEQ ID NO: 5 - RNA, 29 nt, Artificial Sequence
  miR-802-5p
gacgaucucaguaacaaagauucauccuu

SEQ ID NO: 6 - RNA, 25 nt, Artificial Sequence
  miR-802-5p
ucaguaacaaagauucauccuuguu

SEQ ID NO: 7 - RNA, 24 nt, Artificial Sequence
  miR-802-5p
ucaguaacaaagauucauccuugg

SEQ ID NO: 8 - RNA, 24 nt, Artificial Sequence
  miR-802-5p
ucaguaacaaagauucauccuuau

SEQ ID NO: 9 - RNA, 24 nt, Artificial Sequence
  miR-802-5p
ucaguaacaaagauucauccuuga

SEQ ID NO: 10 - RNA, 22 nt, Artificial Sequence
  miR-802-5p
ucaguaacaaagauucauccuu

SEQ ID NO: 11 - RNA, 23 nt, Artificial Sequence
  miR-802-5p
ucaguaacaaagauucauccuua

SEQ ID NO: 12 - RNA, 23 nt, Artificial Sequence
  miR-802-5p
ucaguaacaaagauucauccuuu

SEQ ID NO: 13 - RNA, 26 nt, Artificial Sequence
  miR-802-5p
ucaguaacaaagauucauccuuggau

SEQ ID NO: 14 - RNA, 25 nt, Artificial Sequence
  miR-802-5p
ucaguaacaaagauucauccuugac

SEQ ID NO: 15 - RNA, 29 nt, Artificial Sequence
  miR-802-5p
cccguggucaguaacaaagauucauccuu

SEQ ID NO: 16 - RNA, 24 nt, Artificial Sequence
  miR-802-5p
ucaguaacaaagauucauccuugc

SEQ ID NO: 17 - RNA, 24 nt, Artificial Sequence
  miR-802-5p
cucaguaacaaagauucauccuug

SEQ ID NO: 18 - RNA, 25 nt, Artificial Sequence
  miR-802-5p
ucaguaacaaagauucauccuuagu

SEQ ID NO: 19 - RNA, 49 nt, Artificial Sequence
  miR-802-5p
ucaguaacaaagauucauccuuggaauucucgggugcaaggaacuccag

SEQ ID NO: 20 - RNA, 26 nt, Artificial Sequence
  miR-802-5p
ucaguaacaaagauucauccuugugu

SEQ ID NO: 21 - RNA, 24 nt, Artificial Sequence
  miR-802-5p
ucaguaacaaagauucauccuugu

SEQ ID NO: 22 - RNA, 23 nt, Artificial Sequence
  miR-802-5p
ucaguaacaaagauucauccuug

SEQ ID NO: 23 - RNA, 58 nt, Artificial Sequence
  miR-802-5p
uuugcaaucaguaacaaagauucauccuugugucaaucauacaacacggagagucuuu

SEQ ID NO: 24 - RNA, 22 nt, Artificial Sequence
  miR-802-5p Genome Matched
ucaguaacaaagauucauccuu

SEQ ID NO: 25 - RNA, 23 nt, Artificial Sequence
  miR-802-5p Genome Matched
ucaguaacaaagauucauccuug

SEQ ID NO: 26 - RNA, 24 nt, Artificial Sequence
  miR-802-5p Genome Matched
ucaguaacaaagauucauccuugu

SEQ ID NO: 27 - RNA, 26 nt, Artificial Sequence
  miR-802-5p Genome Matched
ucaguaacaaagauucauccuugugu

SEQ ID NO: 28 - RNA, 23 nt, Artificial Sequence
  miR-802-5p Genome Matched
aucaguaacaaagauucauccuu

SEQ ID NO: 29 - RNA, 24 nt, Artificial Sequence
  miR-802-5p Genome Matched
aucaguaacaaagauucauccuug

SEQ ID NO: 30 - RNA, 25 nt, Artificial Sequence
  miR-802-5p Genome Matched
aucaguaacaaagauucauccuugu

SEQ ID NO: 31 - RNA, 30 nt, Artificial Sequence
  miR-802-5p Genome Matched
aucaguaacaaagauucauccuugugucaa

SEQ ID NO: 32 - RNA, 22 nt, Artificial Sequence
  miR-802-5p U Addition
ucaguaacaaagauucauccuu

SEQ ID NO: 33 - RNA, 23 nt, Artificial Sequence
  miR-802-5p U Addition
ucaguaacaaagauucauccuuu

SEQ ID NO: 34 - RNA, 23 nt, Artificial Sequence
  miR-802-5p U Addition
ucaguaacaaagauucauccuug

SEQ ID NO: 35 - RNA, 24 nt, Artificial Sequence
  miR-802-5p U Addition
ucaguaacaaagauucauccuugu

SEQ ID NO: 36 - RNA, 25 nt, Artificial Sequence
  miR-802-5p U Addition
ucaguaacaaagauucauccuuguu

SEQ ID NO: 37 - RNA, 26 nt, Artificial Sequence
  miR-802-5p U Addition
ucaguaacaaagauucauccuugugu

SEQ ID NO: 38 - RNA, 23 nt, Artificial Sequence
  miR-802-5p U Addition
aucaguaacaaagauucauccuu

SEQ ID NO: 39 - RNA, 24 nt, Artificial Sequence
  miR-802-5p U Addition
aucaguaacaaagauucauccuug

SEQ ID NO: 40 - RNA, 25 nt, Artificial Sequence
  miR-802-5p U Addition
aucaguaacaaagauucauccuugu

SEQ ID NO: 41 - RNA, 30 nt, Artificial Sequence; identical to SEQ ID NO: 31
  miR-802-5p U Addition

SEQ ID NO: 42 - RNA, 22 nt, Artificial Sequence
  miR-802-5p A Addition
ucaguaacaaagauucauccuu

SEQ ID NO: 43 - RNA, 23 nt, Artificial Sequence
  miR-802-5p A Addition
ucaguaacaaagauucauccuua

SEQ ID NO: 44 - RNA, 23 nt, Artificial Sequence
  miR-802-5p A Addition
ucaguaacaaagauucauccuug

SEQ ID NO: 45 - RNA, 24 nt, Artificial Sequence
  miR-802-5p A Addition
ucaguaacaaagauucauccuuga

SEQ ID NO: 46 - RNA, 24 nt, Artificial Sequence
  miR-802-5p A Addition
ucaguaacaaagauucauccuugu

SEQ ID NO: 47 - RNA, 26 nt, Artificial Sequence
  miR-802-5p A Addition
ucaguaacaaagauucauccuugugu

SEQ ID NO: 48 - RNA, 23 nt, Artificial Sequence
  miR-802-5p A Addition
aucaguaacaaagauucauccuu

SEQ ID NO: 49 - RNA, 24 nt, Artificial Sequence
  miR-802-5p A Addition
aucaguaacaaagauucauccuug

SEQ ID NO: 50 - RNA, 25 nt, Artificial Sequence
  miR-802-5p A Addition
aucaguaacaaagauucauccuugu

SEQ ID NO: 51 - RNA, 30 nt, Artificial Sequence; identical to SEQ ID NO: 31
  miR-802-5p A Addition

SEQ ID NO: 52 - RNA, 25 nt, Artificial Sequence
  miR-107-3p
agcagcauuguacagggcuaucacu

SEQ ID NO: 53 - RNA, 23 nt, Artificial Sequence
  miR-107-3p
agcagcauuguacagggcuauca

SEQ ID NO: 54 - RNA, 26 nt, Artificial Sequence
  miR-107-3p
agcagcauuguacagggcuaucaaug

SEQ ID NO: 55 - RNA, 25 nt, Artificial Sequence
  miR-107-3p
agcagcauuguacagggcuaucagu

SEQ ID NO: 56 - RNA, 25 nt, Artificial Sequence
  miR-107-3p
agcagcauuguacagggcuaucauu

SEQ ID NO: 57 - RNA, 26 nt, Artificial Sequence
  miR-107-3p
agcagcauuguacagggcuaucaaca

SEQ ID NO: 58 - RNA, 25 nt, Artificial Sequence
  miR-107-3p
agcagcauuguacagggcuaucaaa

SEQ ID NO: 59 - RNA, 25 nt, Artificial Sequence
  miR-107-3p
cagcagcauuguacagggcuaucaa

SEQ ID NO: 60 - RNA, 24 nt, Artificial Sequence
  miR-107-3p
agcagcauuguacagggcuaucaa

SEQ ID NO: 61 - RNA, 25 nt, Artificial Sequence
  miR-107-3p
agcagcauuguacagggcuaucaga

SEQ ID NO: 62 - RNA, 27 nt, Artificial Sequence
  miR-107-3p
aggagcauuguacagggcuaucaaucu

SEQ ID NO: 63 - RNA, 30 nt, Artificial Sequence
  miR-107-3p
agcagcauuguacagggcuaucaagauuuu

SEQ ID NO: 64 - RNA, 24 nt, Artificial Sequence
  miR-107-3p
agcagcauuguacagggcuaucau

SEQ ID NO: 65 - RNA, 25 nt, Artificial Sequence
  miR-107-3p
agcagcauuguacagggcuaucaau

SEQ ID NO: 66 - RNA, 25 nt, Artificial Sequence
  miR-107-3p
agcagcauuguacagggcuaucaag

SEQ ID NO: 67 - RNA, 26 nt, Artificial Sequence
  miR-107-3p
agcagcauuguacagggcuaucaagu

SEQ ID NO: 68 - RNA, 24 nt, Artificial Sequence
  miR-107-3p
agcagcauuguacagggcuaucag

SEQ ID NO: 69 - RNA, 37 nt, Artificial Sequence
  miR-107-3p
aagcagcauuguacagggcuaucaaagcacagagagc

SEQ ID NO: 70 - RNA, 23 nt, Artificial Sequence
  miR-107-3p Genome Matched
agcagcauuguacagggcuauca

SEQ ID NO: 71 - RNA, 25 nt, Artificial Sequence
  miR-107-3p Genome Matched
agcagcauuguacagggcuaucaaa

SEQ ID NO: 72 - RNA, 25 nt, Artificial Sequence
  miR-107-3p Genome Matched
cagcagcauuguacagggcuaucaa

SEQ ID NO: 73 - RNA, 24 nt, Artificial Sequence
  miR-107-3p Genome Matched
agcagcauuguacagggcuaucaa

SEQ ID NO: 74 - RNA, 37 nt, Artificial Sequence; identical to SEQ ID NO: 69
  miR-107-3p Genome Matched

SEQ ID NO: 75 - RNA, 23 nt, Artificial Sequence
  miR-107-3p U Addition
agcagcauuguacagggcuauca

SEQ ID NO: 76 - RNA, 24 nt, Artificial Sequence
  miR-107-3p U Addition
agcagcauuguacagggcuaucau

SEQ ID NO: 77 - RNA, 25 nt, Artificial Sequence
  miR-107-3p U Addition
agcagcauuguacagggcuaucauu

SEQ ID NO: 78 - RNA, 24 nt, Artificial Sequence
  miR-107-3p U Addition
agcagcauuguacagggcuaucaa

SEQ ID NO: 79 - RNA, 25 nt, Artificial Sequence
  miR-107-3p U Addition
agcagcauuguacagggcuaucaau

SEQ ID NO: 80 - RNA, 25 nt, Artificial Sequence
  miR-107-3p U Addition
agcagcauuguacagggcuaucaaa

SEQ ID NO: 81 - RNA, 37 nt, Artificial Sequence; identical to SEQ ID NO: 69
  miR-107-3p U Addition

SEQ ID NO: 82 - RNA, 23 nt, Artificial Sequence
  miR-107-3p A Addition
agcagcauuguacagggcuauca

SEQ ID NO: 83 - RNA, 25 nt, Artificial Sequence
  miR-107-3p A Addition
agcagcauuguacagggcuaucaaa

SEQ ID NO: 84 - RNA, 25 nt, Artificial Sequence
  miR-107-3p A Addition
cagcagcauuguacagggcuaucaa

SEQ ID NO: 85 - RNA, 24 nt, Artificial Sequence
  miR-107-3p A Addition
agcagcauuguacagggcuaucaa

SEQ ID NO: 86 - RNA, 37 nt, Artificial Sequence; identical to SEQ ID NO: 69
  miR-107-3p A Addition

SEQ ID NO: 87 - RNA, 26 nt, Artificial Sequence
  miR-103-3p
agcagcauuguacagggcuaugauau

SEQ ID NO: 88 - RNA, 26 nt, Artificial Sequence
  miR-103-3p
agcagcauuguacagggcuaugaaau

SEQ ID NO: 89 - RNA, 24 nt, Artificial Sequence
  miR-103-3p
agcagcauuguacagggcuaugau

SEQ ID NO: 90 - RNA, 25 nt, Artificial Sequence
  miR-103-3p
agcagcauuguacagggcuaugaca

SEQ ID NO: 91 - RNA, 25 nt, Artificial Sequence
  miR-103-3p
agcagcauuguacagggcuaugauu

SEQ ID NO: 92 - RNA, 23 nt, Artificial Sequence
  miR-103-3p
agcagcauuguacagggcuauga

SEQ ID NO: 93 - RNA, 28 nt, Artificial Sequence
  miR-103-3p
agcagcauuguacagggcuaugauuuuu

SEQ ID NO: 94 - RNA, 26 nt, Artificial Sequence
  miR-103-3p
agcagcauuguacagggcuaugaucu

SEQ ID NO: 95 - RNA, 26 nt, Artificial Sequence
  miR-103-3p
agcagcauuguacagggcuaugaaga

SEQ ID NO: 96 - RNA, 26 nt, Artificial Sequence
  miR-103-3p
cagcagcauuguacagggcuaugauu

SEQ ID NO: 97 - RNA, 25 nt, Artificial Sequence
  miR-103-3p
agcagcauuguacagggcuaugaua

SEQ ID NO: 98 - RNA, 26 nt, Artificial Sequence
  miR-103-3p
agcagcauuguacagggcuaugaauu

SEQ ID NO: 99 - RNA, 27 nt, Artificial Sequence
  miR-103-3p
agcagcauuguacaggccuaugaaaga

SEQ ID NO: 100 - RNA, 27 nt, Artificial Sequence
  miR-103-3p
agcagcauuguacagggcuaugauucu

SEQ ID NO: 101 - RNA, 27 nt, Artificial Sequence
  miR-103-3p
agcagcauuguacagggcuaugagaau

SEQ ID NO: 102 - RNA, 39 nt, Artificial Sequence
  miR-103-3p
agcagcauuguacagggcuaugauggaauucucgggcca

SEQ ID NO: 103 - RNA, 25 nt, Artificial Sequence
  miR-103-3p
agcagcauuguacagggcuaugaug

SEQ ID NO: 104 - RNA, 26 nt, Artificial Sequence
  miR-103-3p
agcagcauuguacagggcuaugaguu

SEQ ID NO: 105 - RNA, 27 nt, Artificial Sequence
  miR-103-3p
agcagcauuguacagggcuaugagcuc

SEQ ID NO: 106 - RNA, 24 nt, Artificial Sequence
  miR-103-3p
agcagcauuguacagggcuaugac

SEQ ID NO: 107 - RNA, 25 nt, Artificial Sequence
  miR-103-3p
agcagcauuguacagggcuaugaga

SEQ ID NO: 108 - RNA, 25 nt, Artificial Sequence
  miR-103-3p
agcagcauuguacagggcuaugagu

SEQ ID NO: 109 - RNA, 26 nt, Artificial Sequence
  miR-103-3p
agcagcauuguacagggcuaugauuu

SEQ ID NO: 110 - RNA, 24 nt, Artificial Sequence
  miR-103-3p
agcagcauuguacagggcuaugag

SEQ ID NO: 111 - RNA, 26 nt, Artificial Sequence
  miR-103-3p
agcagcauuguacagggcuaugaagc

SEQ ID NO: 112 - RNA, 25 nt, Artificial Sequence
  miR-103-3p
agcagcauuguacagggcuaugacu

SEQ ID NO: 113 - RNA, 25 nt, Artificial Sequence
  miR-103-3p
agcagcauuguacagggcuaugaaa

SEQ ID NO: 114 - RNA, 26 nt, Artificial Sequence
  miR-103-3p
agcagcauuguacagggcuaugauaa

SEQ ID NO: 115 - RNA, 24 nt, Artificial Sequence
  miR-103-3p
agcagcauuguacagggcuaugaa

SEQ ID NO: 116 - RNA, 24 nt, Artificial Sequence
  miR-103-3p
cagcagcauuguacagggcuauga

SEQ ID NO: 117 - RNA, 25 nt, Artificial Sequence
  miR-103-3p
agcagcauuguacagggcuaugaau

SEQ ID NO: 118 - RNA, 26 nt, Artificial Sequence
  miR-103-3p
agcagcauuguacagggcuaugagaa

SEQ ID NO: 119 - RNA, 27 nt, Artificial Sequence
  miR-103-3p
agcagcauuguacagggcuaugauuuu

SEQ ID NO: 120 - RNA, 25 nt, Artificial Sequence
  miR-103-3p
agcagcauuguacagggcuaugauc

SEQ ID NO: 121 - RNA, 27 nt, Artificial Sequence
  miR-103-3p
agcagcauuguacagggcuaugaugcu

SEQ ID NO: 122 - RNA, 36 nt, Artificial Sequence
  miR-103-1
aagcagcauuguacagggcuaugaaggcauugagac

SEQ ID NO: 123 - RNA, 36 nt, Artificial Sequence
  miR-103-2
aagcagcauuguacagggcuaugaaagaaccaagaa

SEQ ID NO: 124 - RNA, 23 nt, Artificial Sequence
  miR-103-3p Genome Matched
agcagcauuguacagggcuauga

SEQ ID NO: 125 - RNA, 24 nt, Artificial Sequence
  miR-103-3p Genome Matched
agcagcauuguacagggcuaugaa

SEQ ID NO: 126 - RNA, 25 nt, Artificial Sequence
  miR-103-3p Genome Matched
agcagcauuguacagggcuaugaaa

SEQ ID NO: 127 - RNA, 27 nt, Artificial Sequence
  miR-103-3p Genome Matched
agcagcauuguacagggcuaugaaaga

SEQ ID NO: 128 - RNA, 36 nt, Artificial Sequence; identical to SEQ ID NO: 122
  miR-103-3p Genome Matched

SEQ ID NO: 129 - RNA, 36 nt, Artificial Sequence; identical to SEQ ID NO: 123
  miR-103-3p Genome Matched

SEQ ID NO: 130 - RNA, 23 nt, Artificial Sequence
  miR-103-3p U Addition
agcagcauuguacagggcuauga

SEQ ID NO: 131 - RNA, 24 nt, Artificial Sequence
  miR-103-3p U Addition
agcagcauuguacagggcuaugau

SEQ ID NO: 132 - RNA, 25 nt, Artificial Sequence
  miR-103-3p U Addition
agcagcauuguacagggcuaugauu

SEQ ID NO: 133 - RNA, 26 nt, Artificial Sequence
  miR-103-3p U Addition
agcagcauuguacagggcuaugauuu

SEQ ID NO: 134 - RNA, 27 nt, Artificial Sequence
  miR-103-3p U Addition
agcagcauuguacagggcuaugauuuu

SEQ ID NO: 135 - RNA, 28 nt, Artificial Sequence
  miR-103-3p U Addition
agcagcauuguacagggcuaugauuuuu

SEQ ID NO: 136 - RNA, 24 nt, Artificial Sequence
  miR-103-3p U Addition
agcagcauuguacagggcuaugaa

SEQ ID NO: 137 - RNA, 25 nt, Artificial Sequence
  miR-103-3p U Addition
agcagcauuguacagggcuaugaau

SEQ ID NO: 138 - RNA, 26 nt, Artificial Sequence
  miR-103-3p U Addition
agcagcauuguacagggcuaugaauu

SEQ ID NO: 139 - RNA, 25 nt, Artificial Sequence
  miR-103-3p U Addition
agcagcauuguacagggcuaugaaa

SEQ ID NO: 140 - RNA, 26 nt, Artificial Sequence
  miR-103-3p U Addition
agcagcauuguacagggcuaugaaau

SEQ ID NO: 141 - RNA, 27 nt, Artificial Sequence
  miR-103-3p U Addition
agcagcauuguacagggcuaugaaaga

SEQ ID NO: 142 - RNA, 36 nt, Artificial Sequence; identical to SEQ ID NO: 122
  miR-103-3p U Addition

SEQ ID NO: 143 - RNA, 36 nt, Artificial Sequence; identical to SEQ ID NO: 123
  miR-103-3p U Addition

SEQ ID NO: 144 - RNA, 23 nt, Artificial Sequence
  miR-103-3p A Addition
agcagcauuguacagggcuauga

SEQ ID NO: 145 - RNA, 24 nt, Artificial Sequence
  miR-103-3p A Addition
agcagcauuguacagggcuaugaa

SEQ ID NO: 146 - RNA, 25 nt, Artificial Sequence
  miR-103-3p A Addition
agcagcauuguacagggcuaugaaa

SEQ ID NO: 147 - RNA, 26 nt, Artificial Sequence
  miR-103-3p A Addition
agcagcauuguacagggcuaugaaga

SEQ ID NO: 148 - RNA, 27 nt, Artificial Sequence
  miR-103-3p A Addition
agcagcauuguacagggcuaugaaaga

SEQ ID NO: 149 - RNA, 36 nt, Artificial Sequence; identical to SEQ ID NO: 122
  miR-103-3p A Addition

SEQ ID NO: 150 - RNA, 36 nt, Artificial Sequence; identical to SEQ ID NO: 123
  miR-103-3p A Addition

SEQ ID NO: 151 - DNA, 20 bp, Artificial Sequence
  Beta-actin - Forward Primer
ggctgtattcccctccatcg

SEQ ID NO: 152 - DNA, 22 bp, Artificial Sequence
  Beta-actin - Reverse Primer
ccagttggtaacaatgccatgt

SEQ ID NO: 153 - DNA, 21 bp, Artificial Sequence
  G6Pase - Forward Primer
cgactcgctatctccaagtga

SEQ ID NO: 154 - DNA, 20 bp, Artificial Sequence
  G6Pase - Reverse Primer
gttgaaccagtctccgacca

SEQ ID NO: 155 - DNA, 18 bp, Artificial Sequence
  Ppar-Gamma - Forward Primer
gcatggtgccttcgctga

SEQ ID NO: 156 - DNA, 22 bp, Artificial Sequence
  Ppar-Gamma - Reverse Primer
tggcatctctgtgtcaaccatg

SEQ ID NO: 157 - DNA, 23 bp, Artificial Sequence
  Ppar-Alpha - Forward Primer
tgtttgtggctgctataatttgc

SEQ ID NO: 158 - DNA, 26 bp, Artificial Sequence
  Ppar-Alpha - Reverse Primer
gcaacttctcaatgtagcctatgttt

SEQ ID NO: 159 - DNA, 22 bp, Artificial Sequence
  Tfam - Forward Primer
cacccagatgcaaaactttcag

SEQ ID NO: 160 - DNA, 23 bp, Artificial Sequence
  Tfam - Reverse Primer
ctgctctttatacttgctcacag

SEQ ID NO: 161 - DNA, 22 bp, Artificial Sequence
  Cs - Forward Primer
gggacttgtgtatgagacttcg

SEQ ID NO: 162 - DNA, 20 bp, Artificial Sequence
  Cs - Reverse Primer
agccaaaataagccctcagg

SEQ ID NO: 163 - DNA, 20 bp, Artificial Sequence
  Cytc - Forward Primer
ggaggcaagcataagactgg

SEQ ID NO: 164 - DNA, 20 bp, Artificial Sequence
  Cytc - Reverse Primer
tccatcagggtatcctctcc

SEQ ID NO: 165 - DNA, 21 bp, Artificial Sequence
  mt-Cox1 - Forward Primer
tccaactcatcccttgacatc

SEQ ID NO: 166 - DNA, 22 bp, Artificial Sequence
  mt-Cox1 - Reverse Primer
tcctgctatgatagcaaacact

SEQ ID NO: 167 - DNA, 21 bp, Artificial Sequence
  mt-Cox2 - Forward Primer
ctaattagctccttagtcctc

SEQ ID NO: 168 - DNA, 21 bp, Artificial Sequence
  mt-Cox2 - Reverse Primer
ttcgtagcttcagtatcattg

SEQ ID NO: 169 - DNA, 21 bp, Artificial Sequence
  mt-Cox3 - Forward Primer
attctattcatcgtctcggaa

SEQ ID NO: 170 - DNA, 21 bp, Artificial Sequence
  mt-Cox3 - Reverse Primer
aaggctatgatgagctcatgt

SEQ ID NO: 171 - DNA, 22 bp, Artificial Sequence
  mt-Atp6 - Forward Primer
taatcaacaaccgtctccattc

SEQ ID NO: 172 - DNA, 21 bp, Artificial Sequence
  mt-Atp6 - Reverse Primer
gtgtcggaagcctgtaattac

SEQ ID NO: 173 - DNA, 21 bp, Artificial Sequence
  mt-Atp8 - Forward Primer
tgccacaactagatacatcaa

SEQ ID NO: 174 - DNA, 21 bp, Artificial Sequence
  mt-Atp8 - Reverse Primer
ggtaatgaatgaggcaaatag

SEQ ID NO: 175 - DNA, 20 bp, Artificial Sequence
  mt-Cytb - Forward Primer
gcaacgaagcctaatattcc

SEQ ID NO: 176 - DNA, 21 bp, Artificial Sequence
  mt-Cytb - Reverse Primer
tgagattggtataagaattaa

SEQ ID NO: 177 - DNA, 21 bp, Artificial Sequence
  mt-Nd1 - Forward Primer
ttaccagaactctactcaact

SEQ ID NO: 178 - DNA, 21 bp, Artificial Sequence
  mt-Nd1 - Reverse Primer
atcgtaacggaagcgtggata

SEQ ID NO: 179 - DNA, 21 bp, Artificial Sequence
  mt-Nd2 - Forward Primer
tcaataattatcctcctggcc

SEQ ID NO: 180 - DNA, 21 bp, Artificial Sequence
  mt-Nd2 - Reverse Primer
atgatagtagagttgagtagc

SEQ ID NO: 181 - DNA, 21 bp, Artificial Sequence
  mt-Nd3 - Forward Primer
ttctagttgcattctgactcc

SEQ ID NO: 182 - DNA, 21 bp, Artificial Sequence
  mt-Nd3 - Reverse Primer
atagaattgtgactagaataa

SEQ ID NO: 183 - DNA, 21 bp, Artificial Sequence
  mt-Nd4 - Forward Primer
gcctgattactgccactaata

SEQ ID NO: 184 - DNA, 21 bp, Artificial Sequence
  mt-Nd4 - Reverse Primer
ggttccctcatcgggtaataa

SEQ ID NO: 185 - DNA, 21 bp, Artificial Sequence
  mt-Nd4L - Forward Primer
actatcacttctagggacact

SEQ ID NO: 186 - DNA, 20 bp, Artificial Sequence
  mt-Nd4L - Reverse Primer
ttggacgtaatctgttccgt

SEQ ID NO: 187 - DNA, 21 bp, Artificial Sequence
  mt-Nd5 - Forward Primer
aaccacacctagcattcctac

SEQ ID NO: 188 - DNA, 21 bp, Artificial Sequence
  mt-Nd5 - Reverse Primer
caggcgttggtgttgcaggta

SEQ ID NO: 189 - DNA, 21 bp, Artificial Sequence
  mt-Nd6 - Forward Primer
acaactatatattgccgctac

SEQ ID NO: 190 - DNA, 21 bp, Artificial Sequence
  mt-Nd6 - Reverse Primer
gatatacgactgctatagcta

SEQ ID NO: 191 - DNA, 19 bp, Artificial Sequence
  Oct1 - Forward Primer
gacgcctggaaagtggacc

SEQ ID NO: 192 - DNA, 23 bp, Artificial Sequence
  Oct1 - Reverse Primer
gcaacatggatgtatagtctggg

SEQ ID NO: 193 - DNA, 22 bp, Artificial Sequence
  Oct3 - Forward Primer
agccagcccgactactattggt

SEQ ID NO: 194 - DNA, 22 bp, Artificial Sequence
  Oct3 - Reverse Primer
tgagctctgagctggtattagt

SEQ ID NO: 195 - DNA, 22 bp, Artificial Sequence
  Gpd2 - Forward Primer
gaaggggactattcttgtgggt

SEQ ID NO: 196 - DNA, 21 bp, Artificial Sequence
  Gpd2 - Reverse Primer
ggatgtcaaattcgggtgtgt

SEQ ID NO: 197 - DNA, 21 bp, Artificial Sequence
  Cav-1 - Forward Primer
atgtctgggggcaaatacgtg

SEQ ID NO: 198 - DNA, 20 bp, Artificial Sequence
  Cav-1 - Reverse Primer
cgcgtcatacacttgcttct

SEQ ID NO: 199 - DNA, 21 bp, Artificial Sequence
  Hnf1Beta - Forward Primer
caccaagccggttttccatac

SEQ ID NO: 200 - DNA, 20 bp, Artificial Sequence
  Hnf1Beta - Reverse Primer
ggagtgtcatagtcgtcgcc

SEQ ID NO: 201 - DNA, 20 bp, Artificial Sequence
  Pgc1a - Forward Primer
cagcctctttgcccagatct

SEQ ID NO: 202 - DNA, 20 bp, Artificial Sequence
  Pgc1a - Reverse Primer
ccgctagcaagtttgcctca

SEQ ID NO: 203 - DNA, 19 bp, Artificial Sequence
  Tnfa - Forward Primer
gctacgacgtgggctacag

SEQ ID NO: 204 - DNA, 23 bp, Artificial Sequence
  Tnfa - Reverse Primer
ccctcacactcagatcatcttct

SEQ ID NO: 205 - DNA, 20 bp, Artificial Sequence
  Srebp1c - Forward Primer
ggagccatggattgcacatt

SEQ ID NO: 206 - DNA, 20 bp, Artificial Sequence
  Srebp1c - Reverse Primer
gcttccagagaggaggccag

SEQ ID NO: 207 - DNA, 22 bp, Artificial Sequence
  Scd1 - Forward Primer
cgggattgaatgttcttgtcgt

SEQ ID NO: 208 - DNA, 22 bp, Artificial Sequence
  Scd1 - Reverse Primer
ttcttgcgatacactctggtgc

SEQ ID NO: 209 - DNA, 20 bp, Artificial Sequence
  Fasn - Forward Primer
aaggctgggctctatggatt

SEQ ID NO: 210 - DNA, 20 bp, Artificial Sequence
  Fasn - Reverse Primer
ggagtgaggctgggttgata

SEQ ID NO: 211 - DNA, 20 bp, Artificial Sequence
  Ampka1 - Forward Primer
tgttccagcagatcctttcc

SEQ ID NO: 212 - DNA, 20 bp, Artificial Sequence
  Ampka1 - Reverse Primer
ataattgggtgagccacagc

SEQ ID NO: 213 - DNA, 22 bp, Artificial Sequence
  Ampka2 - Forward Primer
gggtgaagatcggacactacgt

SEQ ID NO: 214 - DNA, 22 bp, Artificial Sequence
  Ampka2 - Reverse Primer
gtgttctccaatcttcactttg

SEQ ID NO: 215 - DNA, 20 bp, Artificial Sequence
  Ampkb1 - Forward Primer
catcctcccgccacacctgc

SEQ ID NO: 216 - DNA, 20 bp, Artificial Sequence
  Ampkb1 - Reverse Primer
gagcaccatcactccatcct

SEQ ID NO: 217 - DNA, 19 bp, Artificial Sequence
  Ampkb2 - Forward Primer
gggaaaggagcacaagatc

SEQ ID NO: 218 - DNA, 19 bp, Artificial Sequence
  Ampkb2 - Reverse Primer
ctgctgccagggtacaaac

SEQ ID NO: 219 - DNA, 20 bp, Artificial Sequence
  Idh3b - Forward Primer
atctgagcgaggtgcagaat

SEQ ID NO: 220 - DNA, 20 bp, Artificial Sequence
  Idh3b - Reverse Primer
tacgttggcaaacaaatcca

SEQ ID NO: 221 - DNA, 20 bp, Artificial Sequence
  Fh1 - Forward Primer
agcaatgcatattgctgctg

SEQ ID NO: 222 - DNA, 20 bp, Artificial Sequence
  Fh1 - Reverse Primer
cgcatactggacttgctgaa

SEQ ID NO: 223 - DNA, 20 bp, Artificial Sequence
  Mdh1 - Forward Primer
gaagccctgaaagacgacag

SEQ ID NO: 224 - DNA, 20 bp, Artificial Sequence
  Mdh1 - Reverse Primer
tcgacacgaactctccctct

SEQ ID NO: 225 - DNA, 20 bp, Artificial Sequence
  Cptla - Forward Primer
gctgggctactcagaggatg

SEQ ID NO: 226 - DNA, 20 bp, Artificial Sequence
  Cpt1a - Reverse Primer
cactgtagcctggtgggttt

SEQ ID NO: 227 - DNA, 20 bp, Artificial Sequence
  Pdhb - Forward Primer
tcgaagccatagaagccagt

SEQ ID NO: 228 - DNA, 20 bp, Artificial Sequence
  Pdhb - Reverse Primer
aggcatagggacatcagcac

SEQ ID NO: 229 - DNA, 20 bp, Artificial Sequence
  Acss1 - Forward Primer
accagatcctggtggtgaag

SEQ ID NO: 230 - DNA, 20 bp, Artificial Sequence
  Acss1 - Reverse Primer
tcctccagggtagtggtgtc

SEQ ID NO: 231 - DNA, 21 bp, Artificial Sequence
  Acss2 - Forward Primer
gcttctttcccattcttcggt

SEQ ID NO: 232 - DNA, 20 bp, Artificial Sequence
  Acss2 - Reverse Primer
cccggactcattcaggattg

SEQ ID NO: 233 - DNA, 21 bp, Artificial Sequence
  Acly - Forward Primer
gatgaagtggcacctgcaaag

SEQ ID NO: 234 - DNA, 21 bp, Artificial Sequence
  Acly - Reverse Primer
ggtatgtcggctgaagagggt

SEQ ID NO: 235 - DNA, 20 bp, Artificial Sequence
  Sod1 - Forward Primer
ccagtgcaggacctcatttt

SEQ ID NO: 236 - DNA, 20 bp, Artificial Sequence
  Sod1 - Reverse Primer
ttgtttctcatggaccacca

SEQ ID NO: 237 - DNA, 20 bp, Artificial Sequence
  Sod2 - Forward Primer
ccgaggagaagtaccacgag

SEQ ID NO: 238 - DNA, 20 bp, Artificial Sequence
  Sod2 - Reverse Primer
gcttgatagcctccagcaac

SEQ ID NO: 239 - DNA, 22 bp, Artificial Sequence
  Il1b - Forward Primer
gcaactgttcctgaactcaact

SEQ ID NO: 240 - DNA, 21 bp, Artificial Sequence
  Il1b - Reverse Primer
atcttttggggtcgctcaact

SEQ ID NO: 241 - DNA, 20 bp, Artificial Sequence
  Atf4 - Forward Primer
ccttcgaccagtcgggtttg

SEQ ID NO: 242 - DNA, 20 bp, Artificial Sequence
  Atf4 - Reverse Primer
ctgtcccggaaaaggcatcc

SEQ ID NO: 243 - DNA, 24 bp, Artificial Sequence
  mtDNA - Forward Primer
cccagctactaccatcattcaagt

SEQ ID NO: 244 - DNA, 24 bp, Artificial Sequence
  mtDNA - Reverse Primer
gatggtttgggagattggttgatg

SEQ ID NO: 245 - DNA, 20 bp, Artificial Sequence
  5SrRNA - Forward Primer
ggccataccaccctgaacgc

SEQ ID NO: 246 - DNA, 20 bp, Artificial Sequence
  5SrRNA - Reverse Primer
cagcacccggtattcccagg

SEQ ID NO: 247 - DNA, 26 bp, Artificial Sequence
  12SrRNA - Forward Primer
caaactgggattagataccccactat

SEQ ID NO: 248 - DNA, 20 bp, Artificial Sequence
  12SrRNA - Reverse Primer
gagggtgacgggcggtgtgt

SEQ ID NO: 249 - RNA, 22 nt, Artificial Sequence
  miR-802 - Primer
ucaguaacaaagauucauccuu

SEQ ID NO: 250 - RNA, 23 nt, Artificial Sequence
  miR-107 - Primer
agcagcauuguacagggcuauca

SEQ ID NO: 251 - RNA, 22 nt, Artificial Sequence
  miR-320 - Primer
aaaagcuggguugagagggcga

SEQ ID NO: 252 - RNA, 23 nt, Artificial Sequence
  miR-29B - Primer
uagcaccauuugaaaucaguguu

SEQ ID NO: 253 - RNA, 22 nt, Artificial Sequence
  miR-33 - Primer
caauguuuccacagugcaucac

SEQ ID NO: 254 - RNA, 22 nt, Artificial Sequence
  miR-93 - Primer
acugcugagcuagcacuucccg

SEQ ID NO: 255 - RNA, 23 nt, Artificial Sequence
  miR-103 - Primer
agcagcauuguacagggcuauga

SEQ ID NO: 256 - RNA, 22 nt, Artificial Sequence
  miR-130a - Primer
cagugcaauguuaaaagggcau

SEQ ID NO: 257 - RNA, 21 nt, Artificial Sequence
  miR-27b - Primer
uucacaguggcuaaguucugc

SEQ ID NO: 258 - RNA, 21 nt, Artificial Sequence
  miR-152 - Primer
ucagugcaugacagaacuugg

SEQ ID NO: 259 - RNA, 22 nt, Artificial Sequence
  miR-148a - Primer
ucagugcacuacagaacuuugu

SEQ ID NO: 260 - RNA, 22 nt, Artificial Sequence
  miR-148b - Primer
ucagugcaucacagaacuuugu

SEQ ID NO: 261 - DNA, 48 bp, Artificial Sequence
  snoRNA202 - Primer
gctgtactgacttgatgaaagtacttttgaacccttttccatctgatg

SEQ ID NO: 262 - RNA, 22 nt, Artificial Sequence
  pri-miR-802 - Primer
ucaguaacaaagauucauccuu

SEQ ID NO: 263 - RNA, 23 nt, Artificial Sequence
  pri-miR-107 - Primer
agcagcauuguacagggcuauca

SEQ ID NO: 264 - RNA, 23 nt, Artificial Sequence
  pri-miR-103-1 - Primer
agcagcauuguacagggcuauga

SEQ ID NO: 265 - RNA, 23 nt, Artificial Sequence
  pri-miR-103-2 - Primer
agcagcauuguacagggcuauga

SEQ ID NO: 266 - RNA, 22 nt, Artificial Sequence
  pri-mrR-130a - Primer
gcucuuuucacauugugcuacu

SEQ ID NO: 267 - RNA, 24 nt, Artificial Sequence
  pri-miR-152 - Primer
uagguucugugauacacuccgacu

SEQ ID NO: 268 - RNA, 22 nt, Artificial Sequence
  pri-miR-148a - Primer
ucagugcacuacagaacuuugu

SEQ ID NO: 269 - RNA, 24 nt, Artificial Sequence
  pri-miR-148b - Primer
gaaguucuguuauacacucaggcu

SEQ ID NO: 270 - DNA, 21 bp, Artificial Sequence
  Adapter Sequence 1
tggaattctcgggtgccaagg

SEQ ID NO: 271 - DNA, 20 bp, Artificial Sequence
  Adapter Sequence 2
agatcggaagagcacacgtc

SEQ ID NO: 272 - DNA, 28 bp, Artificial Sequence
  Primer
cccagaattccatttaagttacagcctg

SEQ ID NO: 273 - DNA, 32 bp, Artificial Sequence
  Primer
gcatctcgaggttcctttcatgagaaatcaac

SEQ ID NO: 274 - DNA, 60 bp, Artificial Sequence
  Primer
catgatagcttgcataaaagatgacgctatagtttaacgtctgatttccggacaaaaatg

SEQ ID NO: 275 - DNA, 60 bp, Artificial Sequence
  Primer
catttttgtccggaaatcagacgttaaactatagcgtcatcttttatgcaagctatcatg

SEQ ID NO: 276 - RNA, 27 nt, Artificial Sequence
  Ampka1 3′ UTR - 2056-2082 Human
acuucuagaaucaugcacugaauucaa

SEQ ID NO: 277 - RNA, 27 nt, Artificial Sequence
  Ampka1 3′ UTR - 2056-2082 Chimp
acuucuagaaucaugcacugaauucaa

SEQ ID NO: 278 - RNA, 27 nt, Artificial Sequence
  Ampka1 3′ UTR - 2056-2082 Rhesus
acuucuagaaucaugcacugaauucaa

SEQ ID NO: 279 - RNA, 27 nt, Artificial Sequence
  Ampka1 3′ UTR - 2056-2082 Squirrel
accucuagacugaugcacugaauugaa

SEQ ID NO: 280 - RNA, 21 nt, Artificial Sequence
  Ampka1 3′ UTR - 2056-2082 Mouse
uaguuuaugcacugauuuccg

SEQ ID NO: 281 - RNA, 21 nt, Artificial Sequence
  Ampka1 3′ UTR - 2056-2082 Rat
caguuuaugcacugauuucug

SEQ ID NO: 282 - RNA, 27 nt, Artificial Sequence
  Ampka1 3′ UTR - 2056-2082 Rabbit
aucucuaaaaucaugcacugaguucau

SEQ ID NO: 283 - RNA, 27 nt, Artificial Sequence
  Ampka1 3′ UTR - 2056-2082 Pig
aucucuagaaauaugcauugaauucaa

SEQ ID NO: 284 - RNA, 27 nt, Artificial Sequence
  Ampka1 3′ UTR - 2056-2082 Cow
aucccuagaaacaugcacugaauucag

SEQ ID NO: 285 - RNA, 27 nt, Artificial Sequence
  Ampka1 3′ UTR - 2056-2082 Dog
accccuagaaacuugcacugaauucaa

SEQ ID NO: 286 - RNA, 27 nt, Artificial Sequence
  Ampka1 3′ UTR - 2056-2082 Elephant
accccuagaaacaugcacugaauccaa

SEQ ID NO: 287 - RNA, 27 nt, Artificial Sequence
  Ampka1 3′ UTR - 2056-2082 Consensus
accucuagaaacaugcacugaauucaa

SEQ ID NO: 288 - RNA, 21 nt, Artificial Sequence
  miR-152-3p
gguucaagacaguacgugacu

SEQ ID NO: 289 - RNA, 22 nt, Artificial Sequence
  miR-148a-3p
uguuucaagacaucacgugacu

SEQ ID NO: 290 - RNA, 22 nt, Artificial Sequence
  miR-148b-3p
uguuucaagacacuacgucacu

SEQ ID NO: 291 - RNA, 24 nt, Artificial Sequence
  Ampka1 3′ UTR - 2435-2458 Human
auucauuuuaguuacuguguuuua

SEQ ID NO: 292 - RNA, 24 nt, Artificial Sequence
  Ampka1 3′ UTR - 2435-2458  Chimp
auucauuuuaguuacuguguuuua

SEQ ID NO: 293 - RNA, 24 nt, Artificial Sequence
  Ampka1 3′ UTR - 2435-2458 Rhesus
auucaguuuaguuacugcauuuua

SEQ ID NO: 294 - RNA, 24 nt, Artificial Sequence
  Ampka1 3′ UTR - 2435-2458 Squirrel
auucauuuuacuuauugagauuua

SEQ ID NO: 295 - RNA, 24 nt, Artificial Sequence
  Ampka1 3′ UTR - 2435-2458 Mouse
auuccuuuuaguuacugaguuuua

SEQ ID NO: 296 - RNA, 24 nt, Artificial Sequence
  Ampka1 3′ UTR - 2435-2458 Rat
auuccuuuuaguuacugaguuuua

SEQ ID NO: 297 - RNA, 24 nt, Artificial Sequence
  Ampka1 3′ UTR - 2435-2458 Rabbit
auuacuuuuaguuacugauuuuua

SEQ ID NO: 298 - RNA, 24 nt, Artificial Sequence
  Ampka1 3′ UTR - 2435-2458 Pig
auuccuuuuaguuacugaguuuua

SEQ ID NO: 299 - RNA, 24 nt, Artificial Sequence
  Ampka1 3′ UTR - 2435-2458 Cow
auuccuuuaaguuacugaguuuua

SEQ ID NO: 300 - RNA, 24 nt, Artificial Sequence
  Ampka1 3′ UTR - 2435-2458 Dog
auuccuuuuaguuacugaauuuua

SEQ ID NO: 301 - RNA, 23 nt, Artificial Sequence
  Ampka1 3′ UTR - 2435-2458 Elephant
auucuuuuaguuacugagcuuua

SEQ ID NO: 302 - RNA, 24 nt, Artificial Sequence
  Ampka1 3′ UTR - 2435-2458 Consensus
auuccuuuuaguuacugaguuuua

SEQ ID NO: 303 - RNA, 24 nt, Artificial Sequence
  miR-802-5p
uguuccuacuuagaaacaaugacu

SEQ ID NO: 304 - RNA, 24 nt, Artificial Sequence
  Ampka1 3′ UTR - 2467-2490 Human
auauuuaaauauugcacuucuaua

SEQ ID NO: 305 - RNA, 24 nt, Artificial Sequence
  Ampka1 3′ UTR - 2467-2490 Chimp
auauuuaaauauugcacuucuaua

SEQ ID NO: 306 - RNA, 24 nt, Artificial Sequence
  Ampka1 3′ UTR - 2467-2490 Rhesus
auauuuagauauugcacuucuaua

SEQ ID NO: 307 - RNA, 24 nt, Artificial Sequence
  Ampka1 3′ UTR - 2467-2490 Squirrel
auguuuaaauauugcacuuguaca

SEQ ID NO: 308 - RNA, 24 nt, Artificial Sequence
  Ampka1 3′ UTR - 2467-2490 Mouse
acauuaaaauauugcacuuguaua

SEQ ID NO: 309 - RNA, 24 nt, Artificial Sequence
  Ampka1 3′ UTR - 2467-2490 Rat
auauuuaaauauugcacuuguaua

SEQ ID NO: 310 - RNA, 23 nt, Artificial Sequence
  Ampka1 3′ UTR - 2467-2490 Rabbit
uauuuaaauauugcacuuguaca

SEQ ID NO: 311 - RNA, 24 nt, Artificial Sequence
  Ampka1 3′ UTR - 2467-2490 Pig
auauuuaaauauugcacuuguaca

SEQ ID NO: 312 - RNA, 24 nt, Artificial Sequence
  Ampka1 3′ UTR - 2467-2490 Cow
auacuuaaauauugcacuuguuca

SEQ ID NO: 313 - RNA, 24 nt, Artificial Sequence
  Ampka1 3′ UTR - 2467-2490 Dog
auauuuaaauauugcacuuguaca

SEQ ID NO: 314 - RNA, 24 nt, Artificial Sequence
  Ampka1 3′ UTR - 2467-2490 Elephant
auguuucaauauugcacuuguaca

SEQ ID NO: 315 - RNA, 24 nt, Artificial Sequence
  Ampka1 3′ UTR - 2467-2490 Consensus
auauuuaaauauugcacuuguaca

SEQ ID NO: 316 - RNA, 22 nt, Artificial Sequence
  miR-130a-3p
uacgggaaaauuguaacgugac

SEQ ID NO: 317 - RNA, 24 nt, Artificial Sequence
  Ampka1 3′ UTR - 2768-2791 Human
gccagaaauguacuguauacauag

SEQ ID NO: 318 - RNA, 24 nt, Artificial Sequence
  Ampka1 3′ UTR - 2768-2791 Chimp
gccggaaauguacuguauacauag

SEQ ID NO: 319 - RNA, 24 nt, Artificial Sequence
  Ampka1 3′ UTR - 2768-2791 Rhesus
gccagaaauguacuguauacauag

SEQ ID NO: 320 - RNA, 24 nt, Artificial Sequence
  Ampka1 3′ UTR - 2768-2791 Squirrel
gccagaaaaguacuguauacauag

SEQ ID NO: 321 - RNA, 24 nt, Artificial Sequence
  Ampka1 3′ UTR - 2768-2791 Mouse
gccagaaaaguacuguauacauag

SEQ ID NO: 322 - RNA, 24 nt, Artificial Sequence
  Ampka1 3′ UTR - 2768-2791 Rat
gccagaaauguacuguauacauag

SEQ ID NO: 323 - RNA, 24 nt, Artificial Sequence
  Ampka1 3′ UTR - 2768-2791 Rabbit
gccagaaauguacuguauacauag

SEQ ID NO: 324 - RNA, 24 nt, Artificial Sequence
  Ampka1 3′ UTR - 2768-2791 Pig
gccagaaauguacuguauacauag

SEQ ID NO: 325 - RNA, 22 nt, Artificial Sequence
  Ampka1 3′ UTR - 2768-2791 Cow
gccagaaauguacuguacacag

SEQ ID NO: 326 - RNA, 24 nt, Artificial Sequence
  Ampka1 3′ UTR - 2768-2791 Dog
gccagaaauguacuguauacauag

SEQ ID NO: 327 - RNA, 24 nt, Artificial Sequence
  Ampka1 3′ UTR - 2768-2791 Elephant
gcaagaaauguacuguauucauag

SEQ ID NO: 328 - RNA, 24 nt, Artificial Sequence
  Ampka1 3′ UTR - 2768-2791 Consensus
gccagaaauguacuguauacauag

SEQ ID NO: 329 - RNA, 19 nt, Artificial Sequence
  miR-101b-3p
ucgauagugucaugacaug

SEQ ID NO: 330 - RNA, 20 nt, Artificial Sequence
  Cs 3′ UTR - 102-121 Human
aucugaggcacugauaauau

SEQ ID NO: 331 - RNA, 20 nt, Artificial Sequence
  Cs 3′ UTR - 102-121 Chimp
aucugaggcacugauaauau

SEQ ID NO: 332 - RNA, 20 nt, Artificial Sequence
  Cs 3′ UTR - 102-121 Rhesus
aucugaggcacugauaauau

SEQ ID NO: 333 - RNA, 21 nt, Artificial Sequence
  Cs 3′ UTR - 102-121 Squirrel
aucugaggcacugaaaauaac

SEQ ID NO: 334 - RNA, 20 nt, Artificial Sequence
  Cs 3′ UTR - 102-121 Mouse
aucugaggcacugaaaauau

SEQ ID NO: 335 - RNA, 20 nt, Artificial Sequence
  Cs 3′ UTR - 102-121 Rat
aucugaggcacugaaaauac

SEQ ID NO: 336 - RNA, 20 nt, Artificial Sequence
  Cs 3′ UTR - 102-121 Rabbit
aucugaggcacugaaaauau

SEQ ID NO: 337 - RNA, 20 nt, Artificial Sequence
  Cs 3′ UTR - 102-121 Pig
aucugaggcacugaugauaa

SEQ ID NO: 338 - RNA, 20 nt, Artificial Sequence
  Cs 3′ UTR - 102-121 Cow
aucugaggcacugauaauaa

SEQ ID NO: 339 - RNA, 20 nt, Artificial Sequence
  Cs 3′ UTR - 102-121 Dog
aucugaggcacugauaauac

SEQ ID NO: 340 - RNA, 20 nt, Artificial Sequence
  Cs 3′ UTR - 102-121 Elephant
aucugaggcacugauaauaa

SEQ ID NO: 341 - RNA, 20 nt, Artificial Sequence
  Cs 3′ UTR - 102-121 Consensus
aucugaggcacugauaauaa

SEQ ID NO: 342 - RNA, 21 nt, Artificial Sequence
  miR-152-3p
gguucaagacaguacgugacu

SEQ ID NO: 343 - RNA, 22 nt, Artificial Sequence
  miR-148a-3p
uguuucaagacacuacgugacu

SEQ ID NO: 344 - RNA, 22 nt, Artificial Sequence
  miR-148b-3p
uguuucaagacacuacgugacu

SEQ ID NO: 345 - RNA, 23 nt, Artificial Sequence
  Cs 3′ UTR - 1289-1311 Human
uuggacacacuguaaaccccugg

SEQ ID NO: 346 - RNA, 23 nt, Artificial Sequence
  Cs 3′ UTR - 1289-1311 Chimp
uuggacacacuguaaaccccugg

SEQ ID NO: 347 - RNA, 23 nt, Artificial Sequence
  Cs 3′ UTR - 1289-1311 Rhesus
uuggacacacuguaaaccccugg

SEQ ID NO: 348 - RNA, 23 nt, Artificial Sequence
  Cs 3′ UTR - 1289-1311 Squirrel
auggacacacuguaaaccccugg

SEQ ID NO: 349 - RNA, 23 nt, Artificial Sequence
  Cs 3′ UTR - 1289-1311 Mouse
auggacacacuguaaaccccugg

SEQ ID NO: 350 - RNA, 23 nt, Artificial Sequence
  Cs 3′ UTR - 1289-1311 Rat
aggaacacacuguaaaccccugg

SEQ ID NO: 351 - RNA, 23 nt, Artificial Sequence
  Cs 3′ UTR - 1289-1311 Rabbit
auggacacacuguaaaccccugg

SEQ ID NO: 352 - RNA, 28 nt, Artificial Sequence
  Cs 3′ UTR - 1289-1311 Pig
auggauaauacugucuguaaaccucugg

SEQ ID NO: 353 - RNA, 27 nt, Artificial Sequence
  Cs 3′ UTR - 1289-1311 Cow
auggacacauugucuguaaaccccugg

SEQ ID NO: 354 - RNA, 27 nt, Artificial Sequence
  Cs 3′ UTR - 1289-1311 Dog
auggacacauugucuguaaaccucugg

SEQ ID NO: 355 - RNA, 27 nt, Artificial Sequence
  Cs 3′ UTR - 1289-1311 Elephant
auggauacacugucuguaaaccucugg

SEQ ID NO: 356 - RNA, 23 nt, Artificial Sequence
  Cs 3′ UTR - 1289-1311 Consensus
auggacacacuguaaaccccugg

SEQ ID NO: 357 - RNA, 19 nt, Artificial Sequence
  miR-101b-3p
ucgauagugucaugacaug